(19) United States
(12) Patent Application Publication
(10) Pub. No.: US 2020/0255847 A1
(43) Pub. Date: Aug. 13, 2020

(54) METHOD OF IMPROVING POTEXVIRAL VECTOR STABILITY

(71) Applicant: Nomad Bioscience GmbH, München (DE)

(72) Inventors: Stefano Torti, Halle (Saale) (DE); René Schlesier, Halle (Saale) (DE); Anatoli Giritch, Halle (Saale) (DE); Yuri Gleba, Berlin (DE)

(73) Assignee: Nomad Bioscience GmbH, München (DE)

(21) Appl. No.: 16/646,862

(22) PCT Filed: Sep. 17, 2018
(86) PCT No.: PCT/EP2018/075074

§ 371 Date: Mar. 12, 2020

(30) Foreign Application Priority Data
Sep. 18, 2017 (EP) 17191524.2

(51) Int. Cl. C12N 15/82

(52) U.S. Cl. CPC C12N 15/8216; C12N 2770/00043; C12N 15/8203

(57) ABSTRACT

The invention provides a method of producing a potexviral vector for expressing a protein of interest in a plant, comprising producing a second heterologous nucleic acid comprising a second ORF encoding said protein and having, in the second ORF, an increased GC-content compared to a first ORF encoding said protein in a first heterologous nucleic acid, and providing said potexviral vector comprising the following segments: (i) a nucleic acid sequence segment encoding a potexviral RNA-dependent RNA polymerase, (ii) a nucleic acid sequence comprising or encoding a potexviral triple-gene block, and (iii) said second heterologous nucleic acid or a portion thereof comprising said second ORF.

### FIELD OF THE INVENTION

The present invention relates to a method of producing a potexviral vector for expressing a protein of interest in a plant. The invention also relates to methods of improving the capability for long-distance movement in a plant of a potexviral replicon. The invention also relates to methods of improving the stability of a potexviral replicon. The invention also provides a process of expressing a protein of interest in a plant or in plant tissue. Further, nucleic acids for the methods and processes are provided.

### BACKGROUND OF THE INVENTION

High-yield expression of heterologous proteins in plants can be achieved using viral vectors. Viral vector systems were predominantly developed for transient expression followed by infection (Donson et al., 1991, Proc Natl Acad Sci U S A, 88:7204-7208; Chapman, Kavanagh & Baulcombe, 1992, Plant J., 2:549-557) or transfection (Marillonnet et al., 2005, Nat Biotechnol., 23:718-723; Santi et al., 2006, Proc Natl Acad Sci U S A. 103:861-866; WO2005/049839) of a plant host. The best-established and commercially viable systems are based on plus-sense single-stranded RNA viruses, preferably on Tobacco Mosaic Virus (TMV)-derived vectors. Another group of RNA virus-based vectors derived from potexvirus such as PVX (Potato Virus X) can also provide high yield of recombinant proteins (Chapman, Kavanagh & Baulcombe, 1992, Plant J., 2:549-557; Baulcombe, Chapman & Santa Cruz, 1995, Plant J., 7:1045-1053; Zhou et al., 2006, Appl. MicrobioL Biotechnol., 72 (4): 756-762; Zelada et al., 2006, Tuberculosis, 86:263-267). Also potexviruses are plant RNA viruses with a plus-sense single-stranded genome.

In the first generation of systemic viral vectors, a large proportion of plant resources was wasted for the production of viral coat protein that is necessary for systemic movement of a viral replicon. For TMV-derived vectors this problem was solved by removing the coat protein gene and by using agro-infiltration for efficient systemic delivery of replicons, thus significantly boosting the yield of recombinant proteins of interest (WO2005/049839; Marillonnet et al., (2005), Nat. Biotechnol., 23:718-723). However, unlike for TMV-derived replicons, for potexvirus-derived replicons viral coat protein is preferred not only for systemic, but also for short distance (cell-to-cell) movement. Avesani et al. (2007), Transgenic Res. 16:587-597 describe that the stability of PVX expression vectors is related to insert size. WO 2008/028661 describes a way to increase the expression yield of a protein of interest expressed in a plant or in plant tissue from a potexviral vector by a vector design wherein the sequences as defined in item (ii) of claim 1 are positioned after (downstream in 5 to 3′ direction) the RNA-dependent RNA polymerase coding sequence (RdRp or RdRP) of item (i) and precede said heterologous nucleic acid of item (iii). In the special case of potexviral vectors, this vector design leads to a cell-to-cell movement capability of the RNA replicon and, at the same time, to higher expression levels of the heterologous nucleic acid compared to potexviral vectors where a heterologous nucleic acid was placed upstream of the potexviral coat protein gene.

Viral vectors used for expressing a foreign gene in plants typically contain, apart from the ORF encoding the foreign protein to be expressed, remaining viral ORFs that allow the vector to replicate and to spread in plant tissue or entire plants, such as by cell to cell movement and/or long distance movement in a plant. When expressing a sequence of interest in a plant, the replicated and spreading viral vector is desired to be stable such as not to change the nucleic acid sequence of interest to be expressed.

### SUMMARY OF THE INVENTION

The inventors have observed that when low leaves of young plants that were infiltrated with an agrobacterial suspension carrying vectors encoding potexviral replicons containing an ORF to be expressed, spread over plant organs, but are sometimes not stable and lose the nucleic acid sequence of interest over time. On the other hand, some potexviral vectors containing a heterologous nucleic acid sequence such as that encoding AtFT or sGFP are unusually stable.

It is therefore an object of the present invention to provide a potexviral vector for expressing a heterologous nucleic acid or heterologous protein of interest, that is stable, notably in long-distance movement of the vector in plants.

The inventors have studied this problem in detail to find a solution. Accordingly, the present invention provides:

(1) A method of producing a potexviral vector for expressing a protein of interest in a plant, comprising

producing a second heterologous nucleic acid sequence comprising a second ORF encoding said protein of interest and having, in the second ORF, an increased GC-content compared to a first ORF encoding said protein of interest in a first heterologous nucleic acid sequence, and

providing said potexviral vector comprising the following segments: (i) a nucleic acid sequence encoding a potexviral RNA-dependent RNA polymerase, (ii) a nucleic acid sequence comprising or encoding a potexviral triple-gene block, and (iii) said second heterologous nucleic acid sequence or a portion thereof, said portion comprising said second ORF; said portion may consist of said second ORF.

(2) A method of producing a potexviral vector for expressing a protein of interest in a plant, comprising

producing a second heterologous nucleic acid sequence comprising a second ORF encoding said protein of interest and having, in the second ORF, an increased GC-content compared to a first ORF encoding said protein of interest in a first heterologous nucleic acid sequence, and

providing said potexviral vector comprising the following segments: (i) a nucleic acid sequence encoding a potexviral RNA-dependent RNA polymerase, (ii) a nucleic acid sequence comprising or encoding a potexviral triple-gene block, and (iii) said second ORE

(3) A method of improving the capability for long-distance movement in a plant of a potexviral replicon encoding a protein of interest to be expressed in said plant, comprising

producing a second heterologous nucleic acid sequence comprising a second ORF encoding said protein of interest and having, in the second ORF, an increased GC-content compared to a first ORF encoding said protein of interest in a first heterologous nucleic acid sequence, and

providing said potexviral replicon, or a potexviral vector comprising or encoding said potexviral replicon, said potexviral replicon comprising the following segments: (i) a nucleic acid sequence encoding a potexviral RNA-dependent RNA polymerase, (ii) a nucleic acid sequence comprising a potexviral triple-gene block, and (iii) said second heterologous nucleic acid sequence or a portion thereof, said portion comprising said second ORF; said portion may consist of said second ORE

(4) A method of improving the capability for long-distance movement in a plant of a potexviral replicon encoding a protein of interest to be expressed in said plant, comprising

producing a second heterologous nucleic acid sequence comprising a second ORF encoding said protein of interest and having, in the second ORF, an increased GC-content compared to a first ORF encoding said protein in a first heterologous nucleic acid sequence, and

providing said potexviral replicon, or a potexviral vector comprising or encoding said potexviral replicon, said potexviral replicon comprising the following segments: (i) a nucleic acid sequence encoding a potexviral RNA-dependent RNA polymerase, (ii) a nucleic acid sequence comprising a potexviral triple-gene block, and (iii) said second ORF.

(5) The method according to any one of (1), (2), (3) or (4), wherein said step of providing a potexviral vector or potexviral replicon comprises inserting said second heterologous nucleic acid sequence, or a portion thereof comprising said second ORF, into a nucleic acid comprising (i) a nucleic acid sequence encoding a potexviral RNA-dependent RNA polymerase and (ii) a nucleic acid sequence encoding a potexviral triple-gene block to produce the potexviral vector or the potexviral replicon comprising the second heterologous nucleic acid sequence or a portion thereof comprising said second ORF.

(6) A process of expressing a protein of interest in a plant or in plant tissue, comprising producing a potexviral vector according to the method of (1) or (2) or as further defined in (5) and providing the produced potexviral vector to at least a part of said plant.

(7) The method or process according to any one of (1) to (6), wherein said plant is selected from Nicotiana species such as Nicotiana benthamiana and Nicotiana tabacum, tomato, potato, pepper, eggplant, soybean, Petunia hybrida, Brassica napus, Brassica campestris, Brassica juncea, cress, arugula, mustard, strawberry, spinach, Chenopodium capitatum, alfalfa, lettuce, sunflower, potato, cucumber, corn, wheat, and rice.

(8) The method or process according to any one of (1) to (7), wherein said (ii) nucleic acid sequence comprising or encoding a potexviral triple-gene block further comprises a nucleic acid sequence encoding a potexviral coat protein or a nucleic acid sequence encoding a tobamoviral movement protein.

(9) A method of improving the capability for long-distance movement in a plant of a potexviral replicon encoding a protein of interest to be expressed in said plant, comprising

increasing the GC-content of a first ORF encoding said protein in a first heterologous nucleic acid sequence, thereby obtaining a second heterologous nucleic acid sequence comprising a second ORF, said second ORF encoding said protein and having an increased GC-content, and

inserting said second heterologous nucleic acid sequence, or a portion thereof containing said second ORF, into a nucleic acid comprising (i) a nucleic acid sequence encoding a potexviral RNA-dependent RNA polymerase and (ii) a nucleic acid sequence comprising or encoding a potexviral triple-gene block to produce a potexviral vector comprising or encoding said potexviral replicon, said potexviral vector comprising the second heterologous nucleic acid sequence or a portion thereof, said portion comprising said second ORF.

(10) A potexviral vector obtained or obtainable by the method of (1) or (2), optionally as further defined in (5) and/or (8).

(11) A nucleic acid comprising the following segments: (i) a nucleic acid sequence encoding a potexviral RNA-dependent RNA polymerase, (ii) nucleic acid sequence comprising or encoding a potexviral triple-gene block, and (iii) a heterologous nucleic acid sequence comprising an ORF encoding a protein of interest, wherein

said ORF consists of at least 200 and at most 400 nucleotides and has a GC-content of at least 50%; or

said ORF consists of at least 401 and at most 800 nucleotides has a GC-content of at least 55%; and/or

said ORF consists of at least 801 nucleotides and has a GC-content of at least 58%.

(12) A nucleic acid comprising the following segments: (i) a nucleic acid sequence encoding a potexviral RNA-dependent RNA polymerase, (ii) nucleic acid sequence comprising or encoding a potexviral triple-gene block, and (iii) a heterologous nucleic acid sequence comprising an ORF encoding a protein of interest, wherein

said ORF consists of at least 100 and at most 500 nucleotides and has a GC-content of at least 50%; or

said ORF consists of at least 501 and at most 1000 nucleotides has a GC-content of at least 55%; and/or

said ORF consists of at least 1001 nucleotides and has a GC-content of at least 58%.

(13) The nucleic acid according to (11) or (12), said nucleic acid further comprising a nucleic acid sequence encoding a potexviral coat protein or a nucleic acid sequence encoding a tobamoviral movement protein.

(14) The nucleic acid according to any one of (11) to (13), wherein the protein of interest is not a plant viral protein or it is a protein that is heterologous to plant viruses, preferably said protein of interest is not a potexviral coat protein or a tobamoviral movement protein.

(15) A combination or kit comprising a first and a second nucleic acid, said first nucleic acid comprising segments (i) and (ii) as defined in (11), (12) or (13), said second nucleic acid comprising segment (iii) as defined in (11), (12) or (14).

(16) The combination or kit according to (15), wherein said first nucleic acid has, downstream of segment (ii) a first site-specific recombination site recognizable by a site-specific recombinase, and said second nucleic acid has, upstream of segment (iii), a second site-specific recombination site recognizable by said site-specific recombinase for allowing site-specific recombination between said first and said second site-specific recombination site and formation of a nucleic acid according to (11), (12), (13) or (14), or a potexviral vector according to (10).

(17) A process of expressing a heterologous nucleic acid sequence of interest in a plant or in plant tissue, comprising providing the plant or plant tissue with a nucleic acid of (11) or (12), with a potexviral vector according to (10), or with a combination or kit of nucleic acids according to (15) or (16), for expressing said heterologous nucleic acid sequence of interest.

(18) Use of a heterologous nucleic acid as defined in (11) to (14), a potexviral vector according to (10), or a combination or kit according to (15) or (16) for expressing a protein encoded by said heterologous nucleic acid and for achieving improved long-distance movement of a potexviral vector in a plant.

The inventors have surprisingly found that potexviral replicons carrying a heterologous nucleic acid encoding a protein of interest for expression in a plant or plant tissue have an improved capability for long-distance movement in a plant and/or replicon stability in the plant is improved, if the GC content of the heterologous nucleic acid, notably of the ORF encoding the protein of interest, is increased. Thereby, the expression yield of a protein of interest in the plant or plant tissue is improved and costs for purification of the protein of interest decrease. In one embodiment, the protein of interest provides the plant with an agronomic trait.

### BRIEF DESCRIPTION OF THE DRAWINGS

FIG. 1 shows schematically Potato Virus X (PVX)-based entry vectors pNMD4300 and pNMD670 for cloning of inserts of interest. The nucleotide sequences of these vectors are given as SEQ ID NO: 24 and 23, respectively.

RB and LB indicate the right and left borders of T-DNA of binary vectors. P35S: cauliflower mosaic virus 35S promoter; PVX-pol: RNA-dependent RNA polymerase from PVX; CP: coat protein ORF; 25K, 12K and 8K together indicate the 25 kDa, 12 kDa and 8 kDa triple gene block modules from PVX; N: 3′-untranslated region from PVX. INSERT stands for DNA insert of interest; Bsal stand for Bsal restriction sites with corresponding nucleotide overhangs shown below. virGN54D is a virG gene with N54D mutation from LBA4404 strain of Agrobacterium tumefaciens.

FIG. 2 shows RT-PCR analysis of foreign insert stability in PVX viral vectors.

36 days old tomato Solanum lycopersicum ‘Balcony Red’ plants were transfected by syringe infiltration of agrobacterial cultures carrying PVX vectors. The infiltration was performed into two cotyledons leaves. Total RNA was isolated from systemic leaves of PVX infected plants 26 days post infiltration using NucleoSpin® RNA Plant kit (Macherey-Nagel). RNA was reverse transcribed using PrimeScript™ RT Reagent Kit (Takara Clontech); resulting cDNA was used as a template for PCR with oligos specific for either PVX (UPPER PANEL) or tobacco Elongation Factor EF1α used as a RNA loading control (LOWER PANEL). PCR fragments of expected size are shown with arrows. Positions of missing expected PCR products on the gel are shown with a dashed line.

RT-PCR products were resolved in 1% agarose gels. MWL: Molecular Weight Ladder; GFP: RT-PCR product for plant infected with PVX vector carrying GFP insertion; GUS, AtFT, CaDREB, SISUN, SILOG1, SIDREB1, SIGR, SIOVATE, SIWOOLLY: RT-PCR products for plants infected with PVX vectors with insertions of GUS, AtFT, CaDREB, SISUN, SILOG1, SIDREB1, SIGR, SIOVATE, SIWOOLLY genes, respectively; V: plant infected with empty PVX entry vector without foreign insertion. Sizes of expected PCR fragments are given in brackets.

FIG. 3 shows the relation between Insert Length and Stability. Latest day post infiltration when the full-length insert was detected (Y-axis) was plotted against the length of corresponding foreign insert (X axis). For analysis, values for GFP, GUS, AtFT, CaDREB, SISUN, SILOG1, SIDREB1, SIGR, SIOVATE and SIWOOLLY inserts (Table 1) were used.

FIG. 4 shows the relation between GC content and Stability of the insert. Latest day post infiltration when the full-length insert was detected (Y-axis) was plotted against the GC content (%) of corresponding foreign insert (X axis). For analysis, values for GFP, GUS, AtFT, CaDREB, SISUN, SILOG1, SIDREB1, SIGR, SIOVATE and SIWOOLLY inserts (Table 1) were used. GC content of inserts was determined using ENDMEMO on-line DNA/RNA GC Content Calculator (www.endmemo.com/bio/gc.php).

FIG. 5 shows the relation between GC content to Length Ratio and Stability of the insert. Latest day post infiltration when the full-length insert was detected (Y-axis) was plotted against the GC content to Length Ratio of corresponding foreign insert (X axis). For analysis, values for GFP, GUS, AtFT, CaDREB, SISUN, SILOG1, SIDREB1, SIGR, SIOVATE and SIWOOLLY inserts (Table 1) were used. The ratio between GC content and Length of insert was calculated using the formula: Ratio GC Content/Length=(GC content (%)/Length (bp)×100.

FIG. 6 shows RT-PCR analysis of PVX vector stability for construct containing SIANT1 insertions with different codon usage 21 days post infiltration (dpi). Systemic leaves of three independent tomato ‘Balcony Red’ plants were analyzed as described in Example 2.

Native (35.2%; 4.3): native SIANT1 coding sequence with 35.2% GC content and 4.3 Ratio GC Content/Length (pNMD721 construct).

Tobacco (39.5%; 4.8): SIANT1 coding sequence optimized for Nicotiana tabacum codon usage (39.5% GC content and 4.8 Ratio GC Content/Length; pNMD29561).

Arabidopsis (41.0%; 5.0): SIANT1 coding sequence optimized for Arabidopsis thaliana codon usage (41.0% GC content and 5.0 Ratio GC Content/Length; pNMD29541).

Human (48.0%, 5.8): SIANT1 coding sequence optimized for Homo sapiens codon usage (48.0% GC content and 5.8 Ratio GC Content/Length; pNMD29531).

Rice (48.4%; 5.9): SIANT1 coding sequence optimized for Homo sapiens codon usage (48.4% GC content and 5.9 Ratio GC Content/Length; pNMD29551).

V: empty entry PVX vector pNMD4300. PL: plasmid; 1, 2, and 3: Plants 1, 2, and 3, respectively. Plasmid amplified PCR fragment serves as a positive size control.

FIG. 7 shows RT-PCR analysis of PVX vector stability for construct containing SIANT1 insertions with different codon usage 52 days post infiltration. Systemic leaves of three independent tomato ‘Balcony Red’ plants were analyzed as described in Example 2.

Native (35.2%; 4.3): native SIANT1 coding sequence with 35.2% GC content and 4.3 Ratio GC Content/Length (pNMD721 construct).

Tobacco (39.5%; 4.8): SIANT1 coding sequence optimized for Nicotiana tabacum codon usage (39.5% GC content and 4.8 Ratio GC Content/Length; pNMD29561).

PVX (44.7%; 5.4): SIANT1 coding sequence optimized for PVX codon usage (44.7% GC content and 5.4 Ratio GC Content/Length; pNMD30881).

Barley (51.0%; 6.2): SIANT1 coding sequence optimized for Hordeum vulgare codon usage (51.0% GC content and 6.2 Ratio GC Content/Length; pNMD30722).

Bifido (56.1%; 6.8): SIANT1 coding sequence optimized for Bifidobacterium codon usage (56.1% GC content and 6.8 Ratio GC Content/Length; pNMD30891).

V: empty entry PVX vector pNMD4300. PL: plasmid; 1, 2, and 3: Plants 1, 2, and 3, respectively. Plasmid amplified PCR fragment serves as a positive size control.

FIG. 8 shows RT-PCR analysis of PVX vector stability for construct containing native and codon-optimized sequences of SILOG1 and SIOVATE genes.

(A) Analysis of vectors with SILOG1 insertions.

Plant material from systemic leaves of tomato ‘Balcony Red’ plants was analyzed 34 days post infiltration. 1: plant transfected with pNMD27533 construct containing native SILOG1 sequence (41.9% GC content and 6.2 Ratio GC Content/Length). 2: plant transfected with pNMD31084 construct containing SILOG1 sequence optimized for Oryza sativa codon usage (53.2% GC content and 7.8 Ratio GC Content/Length). Expected size of PCR fragment for intact insertion is 870 bp, shown with arrow.

(B) Analysis of vectors with SILOG1 insertions. Upper panel: plant material analyzed 27 days post infiltration; Lower panel: plant material analyzed 82 days post infiltration.

Native (41.0%; 3.9): native SIOVATE coding sequence with 41.0% GC content and 4.6 Ratio GC Content/Length (pNMD27931 construct).

Rice (48.8%; 4.6): SIOVATE coding sequence optimized for Oryza sativa codon usage (48.8% GC content and 4.6 Ratio GC Content/Length; pNMD29551).

V: empty entry PVX vector pNMD4300. PL: plasmid; 1 and 2: Plants 1 and 2, respectively. Plasmid amplified PCR fragment serves as a positive size control.

FIG. 9 shows Table 1: PVX vector insertions and their stability (Example 2).

FIG. 10 shows Table 3: Native and codon-optimized sequences of SILOG1 and SIOVATE genes (Example 4).

FIG. 11 shows GFP fluorescence in fruits of tomato ‘Balcony Red’ plants inoculated with PVX vectors containing the insertion of sGFP original sequence (FIG. 11, A) and the insertion of sGFP sequence adapted for tobacco codon usage (sGFP-tobacco, FIG. 11, B). Photos were taken 102 days post infiltration. White arrows show fruit areas with GFP fluorescence. For each constructs, two independent plants (Plant 1 and Plant 2) were used (Example 5).

sGFP (61.4%; 8.5): original sGFP coding sequence with 61.4% GC content and 8.5 Ratio GC Content/Length (pNMD5800 construct).

sGFP-tobacco (40.3%; 5.6): sGFP coding sequence with Nicotiana tabacum adapted codon usage (40.3% GC content and 5.6 Ratio GC Content/Length; pNMD32685).

FIG. 12 shows RT-PCR analysis of PVX vector stability for constructs containing sGFP insertions with original (sGFP) and tobacco adapted (sGFP-tobacco) codon usage at 25 dpi (upper panel) and 102 dpi (lower panel). For each construct, two independent tomato ‘Balcony Red’ plants were inoculated. At 25 dpi, systemic leaves of inoculated plants were analyzed. At 102 dpi, mature fruits were used for analysis. The analysis was performed as described in Example 5.

PL: plasmid; 1 and 2: Inoculated plants 1 and 2, respectively. Plasmid amplified PCR fragment served as a positive size control. Black arrows show PCR fragments with a size corresponding to intact non-degraded sGFP insert.

sGFP (61.4%; 8.5): original sGFP coding sequence with 61.4% GC content and 8.5 Ratio GC Content/Length (pNMD5800 construct). sGFP-tobacco (40.3%; 5.6): sGFP coding sequence with Nicotiana tabacum adapted codon usage (40.3% GC content and 5.6 Ratio GC Content/Length; pNMD32685).

### DETAILED DESCRIPTION OF THE INVENTION

Herein, the potexviral replicon is a nucleic acid that is replicated in plant cells and capable of cell-to-cell and long distance movement in a plant and in plant tissue. The potexviral replicon makes use of the replication and, preferably, protein expression system of potexviruses in plants or plant cells. The potexviral replicon may be built on a natural potexvirus e.g. by comprising genetic components from a potexvirus, or by using genetic components suitably altered compared to those of a potexvirus. The potexviral replicon is or comprises an RNA. The potexviral vector of the invention is the vehicle used for providing cells of a plant or of plant tissue with the potexviral replicon. The potexviral replicon may itself be used as the potexviral vector of the invention. However, the potexviral vector may comprise or encode the potexviral replicon. The potexviral vector as well as the nucleic acid mentioned below may be DNA or RNA. If it is RNA, it is or comprises the potexviral replicon; if it is DNA, it encodes the potexviral replicon. If the potexviral vector or said nucleic acid are DNA, segments (i) to (iii) are generally also DNA. If said potexviral vector or said nucleic acid are RNA, segments (i) to (iii) are generally also RNA.

The potexviral replicon is an RNA (generally an RNA molecule) comprising at least the following segments (i) to (iii), preferably in this order in 5′- to 3′-direction:

(i) a nucleic acid sequence encoding a potexviral RNA-dependent RNA polymerase (RdRp);

(ii) a nucleic acid sequence comprising:
(a) a potexvirus triple gene block and
(b) optionally a sequence encoding a potexviral coat protein; or a sequence encoding a tobamoviral movement protein; and

(iii) a heterologous nucleic acid sequence comprising an ORF encoding a protein of interest.

The potexviral vector of the invention is a nucleic acid comprising or encoding the potexviral replicon. Accordingly, the potexviral vector comprises, preferably in this order in 5′- to 3′-direction, the following segments (i) to (iii):

(i) a nucleic acid sequence encoding a potexviral RNA-dependent RNA polymerase (RdRp);

(ii) a nucleic acid sequence:
(a) comprising or encoding a potexvirus triple gene block and
(b) optionally comprising a sequence encoding a potexviral coat protein; or comprising a sequence encoding a tobamoviral movement protein; and

(iii) a heterologous nucleic sequence comprising an ORF encoding a protein of interest.

While the order of segments (i) to (iii), in 5′- to 3′ direction, is preferably from segment (i) to segment (ii) to segment (iii) as given above, the order of segments (a) and (b) of segment (ii) is not particularly limited.This preferred order of segments (i) to (iii) also applies to other embodiments of the invention.

The “nucleic acid” of the above potexviral vector and the “RNA” or “RNA molecule” of the above potexviral replicon are also collectively referred to as “nucleic acid of the invention”. The heterologous nucleic sequence of item (iii) is also referred to herein as “second heterologous nucleic (sequence)” and said ORF is also referred to herein as “second ORF”. These elements and their production are further described below. Herein, an ORF (open reading frame) is the coding nucleic acid sequence of the protein of interest. The ORF consists of the base triplets from and including the start codon to the stop codon, and may include introns. The ORF encodes the protein of interest from its N-terminus to its C-terminus. The protein of interest may include N-terminal or C-terminal peptides that may be cleaved off after translation. Thus, in the invention, the “protein of interest” may be the primary translation product produced in a process of expressing a protein, while the final protein that may be purified after expression of the protein of interest may be modified post-translationally.

A “nucleic acid sequence” or, briefly, “sequence”, generally is a nucleic acid molecule or a nucleic acid segment of a longer nucleic acid molecule. A segment (of a nucleic acid) is a plurality of contiguous bases within a longer nucleic acid molecule. The “nucleic acid sequence” or, briefly, “sequence” may be single-stranded or double-stranded. Similarly, a nucleic acid or nucleic acid molecule may be single-stranded or double-stranded. The first and second heterologous nucleic acid sequences of the invention may also be referred to as first and second heterologous nucleic acid, respectively.

The potexviral replicon can replicate in plant cells due to the presence of the potexviral elements or segments of items (i) and (ii) and optionally further genetic elements of the potexviral replicon. These further genetic elements may also be contained in or encoded in the potexviral vector. Examples of such further genetic elements are 5′- and 3′-untranslated regions and subgenomic promoters.

In the methods of the invention, the second heterologous nucleic acid sequence of item (iii) above is produced. The second heterologous nucleic acid sequence generally encodes the same protein as the first heterologous nucleic acid sequence. The second heterologous nucleic acid sequence differs from the first heterologous nucleic acid sequence in that the ORF of the former has a higher GC content than the ORF of the latter. Higher GC content means that the sum of G and C (guanine and cytosine) bases is higher. Thus, “GC content” herein means a G+C content. The GC content is determined by counting the number of G and C bases in a given nucleic acid. The second heterologous nucleic acid sequence may consist of the ORF or coding sequence of the protein of interest. The coding sequence of the protein of interest is herein also referred to as ORF (open reading frame). Alternatively, the second heterologous nucleic acid sequence may comprise the coding sequence (ORF) of the protein of interest and one or more further nucleotides or nucleotide stretches such as restriction endonuclease site(s) for engineering the potexviral vector or genetic elements for expressing the protein of interest from the potexviral replicon in plants or plant cells. The second heterologous nucleic acid sequence may further contain other genetic elements, e.g. elements used for cloning or for introduction of the second ORF into the potexviral replicon or the potexviral vector. Also if the second heterologous nucleic acid sequence comprises additional nucleotides or sequence stretches or other genetic elements, the GC content defined herein is that of the segment that consists of the coding sequence (ORF) of the protein of interest. Preferably, the second heterologous nucleic acid sequence has a higher GC content than the first heterologous nucleic acid sequence.

The first heterologous nucleic acid sequence also comprises an ORF that encodes the protein of interest. The first heterologous nucleic acid sequence may be a physical entity such as a nucleic acid molecule. However, for the invention, it is sufficient that the higher CG content of the ORF of the second heterologous nucleic acid can be determined by counting GC bases. Therefore, it is not necessary that the first heterologous nucleic acid and its ORF is/are a physical entity; it is sufficient that the first heterologous nucleic acid is a virtual nucleic acid, e.g. represented by the commonly used characters C, G, A, and T/U written on a sheet of paper or written in a computer-readable electronic file. As is generally known, these characters stand for cytosine, guanine, adenine and thymine/uracil nucleotides, respectively, in a nucleic acid sequence.

The method employed for producing the second heterologous nucleic acid is not limited, provided the GC-content of the ORF encoding the protein of interest is higher than that of the ORF encoding the protein of interest of a first heterologous nucleic acid sequence. Methods of producing a nucleic acid are part of the general knowledge in molecular biology. The second heterologous nucleic acid may, for example, be produced by automated DNA synthesis. The second heterologous nucleic acid may, alternatively, be produced by modifying the first heterologous nucleic acid by replacing nucleotides such that the GC content of the ORF encoding the protein of interest increases. Nucleotides of the first heterologous nucleic acid other than of the ORF may, if desired, also be changed in the production of the second heterologous nucleic acid.

Using the produced second heterologous nucleic acid, the potexviral replicon or the potexviral vector may be provided. The methods applicable in this step are generally known methods of molecular biology, and the invention is not limited with regard to the specific method used. Generally, it is preferred and more common to make the necessary nucleic acid modifications on the DNA level. Therefore, it is preferred that the second heterologous nucleic acid sequence is DNA and that the potexviral vector encoding the potexviral replicon is produced. For example, the second heterologous nucleic acid sequence may be inserted into a nucleic acid comprising a nucleic acid comprising the segments (i) and (ii) above to produce the potexviral vector of the invention. The step of inserting the second heterologous nucleic acid sequence may be a usual a sub-cloning step wherein parts or nucleotides of the second heterologous nucleic acid, e.g. nucleotides of an endonuclease restriction site, may get lost, i.e. may not be present in the product. Thus, it is possible that not the entire second heterologous nucleic acid sequence ends up in the potexviral vector. In any event, at least a portion comprising the ORF of the protein of interest of the second heterologous nucleic acid (i.e. said second ORF) is inserted into the product which is the potexviral vector. In another embodiment, the second ORF is inserted into the product which is the potexviral vector, e.g. without additional sequence stretches beyond the second ORF. However, also in this case, the genetic elements necessary for expressing the protein of interest are preferably provided to the potexviral vector.

The second heterologous nucleic acid sequence may, apart from said ORF, further comprise genetic elements for expressing the protein of interest in plants or plants cells from the potexviral replicon, such as a ribosome binding site, a 5′-untranslated region and/or a 3′-untranslated region.

Said portion thereof, i.e. the portion of the second heterologous nucleic acid sequence that comprises or consists of the second ORF, is a (sequence) segment of the second heterologous nucleic acid, that comprises or consists of the second ORF. Said portion may be a product of the second heterologous nucleic acid sequence after digestion with one or two restriction enzymes or endonucleases for insertion of the digestion product into the potexviral vector. The portion may contain genetic elements for expressing the protein of interest in plants or plants cells from the potexviral replicon, as those mentioned in the previous paragraph.

In the following, embodiments of the nucleic acid are described.

The nucleic acid of the invention comprises the following segments: (i) a nucleic acid sequence encoding a potexviral RNA-dependent RNA polymerase, (ii) a nucleic acid sequence comprising or encoding a potexviral triple-gene block, and (iii) a heterologous nucleic acid sequence comprising an ORF encoding the protein of interest.

In one embodiment, said ORF consists of at least 100 and at most 500 nucleotides and has a GC-content of at least 50%; or said ORF consists of at least 501 and at most 1000 nucleotides has a GC-content of at least 55%; and/or said ORF consists of at least 1001 nucleotides and has a GC-content of at least 58%.

In another embodiment, said ORF consists of at least 100 and at most 500 nucleotides and has a GC-content of at least 50%; or said ORF consists of at least 501 and at most 1000 nucleotides has a GC-content of at least 55%; and/or said ORF consists of at least 1001 nucleotides and has a GC-content of at least 58%.

In a further embodiment, said ORF has a GC-content of at least 50% within a segment of said heterologous nucleic acid sequence, said segment consisting of at least 200 and at most 400 nucleotides, preferably at least 100 and at most 500 nucleotides; or said ORF has a GC-content of at least 55% within a segment of said heterologous nucleic acid sequence, said segment consisting of from 401 to 800 nucleotides, preferably from 501 to 1000 nucleotides; and/or said ORF has a GC-content of at least 58% within a segment of said heterologous nucleic acid sequence, said segment consisting of 801 or more, preferably 1001 or more nucleotides. Preferably, said ORF has a GC-content of at least 52% within a segment of said heterologous nucleic acid sequence, said segment consisting of at least 200 and at most 400 nucleotides, preferably at least 100 and at most 500 nucleotides; or said ORF has a GC-content of at least 57% within a segment of said heterologous nucleic acid sequence, said segment consisting of from 401 to 800 nucleotides, preferably from 501 to 1000 nucleotides; and/or said ORF has a GC-content of at least 60% within a segment of said heterologous nucleic acid sequence, said segment consisting of 801 or more, preferably 1001 or more nucleotides. In another embodiment of the nucleic acid, said ORF has a GC-content of at least 50% within a segment of said heterologous nucleic acid sequence, said segment consisting of at least 100 and at most 500 nucleotides; or said ORF has a GC-content of at least 55% within a segment of said heterologous nucleic acid sequence, said segment consisting of from 501 to 1000 nucleotides; and/or said ORF has a GC-content of at least 58% within a segment of said heterologous nucleic acid sequence, said segment consisting of 1001 or more nucleotides; preferably, said ORF has a GC-content of at least 52% within a segment of said heterologous nucleic acid sequence, said segment consisting of at least 100 and at most 500 nucleotides; or said ORF has a GC-content of at least 57% within a segment of said heterologous nucleic acid sequence, said segment consisting of from 501 to 1000 nucleotides; and/or said ORF has a GC-content of at least 60% within a segment of said heterologous nucleic acid sequence, said segment consisting of 1001 or more nucleotides.

Potexviral vectors or nucleic acids comprising a heterologous nucleic acid encoding a green fluorescent protein may be excluded from the potexviral vectors or nucleic acid of the invention, respectively.

Said potexviral vector or nucleic acid of the invention may be obtainable by inserting the second heterologous nucleic acid sequence into a nucleic acid construct encoding a potexvirus, whereby said heterologous nucleic acid sequence may be inserted downstream of a sequence encoding the triple gene block and/or downstream of a sequence encoding the coat protein of said potexvirus. However, modifications may be made to the genetic components of a natural potexvirus, such as to the RdRP gene, the triple gene block, the coat protein gene, or to the 5′ or 3′ non-translated regions of a potexvirus, examples for which are described below.

The potexviral vector of the invention comprises, generally in the order from the 5′ end to the 3′ end, said segments (i) to (iii) of the invention. Further genetic elements may be present on said replicon or vector for replication of the potexviral replicon in plant cells and/or or for expression of the protein of interest. For being a replicon, i.e. for autonomous replication in a plant cell, the potexviral replicon encodes an RdRp. The potexviral replicon may further have potexviral 5′- and/or 3′-untranslated regions and promoter-sequences in the 5′- or 3′-untranslated regions of said potexviral replicon for binding the potexviral RdRp and for replicating the potexviral replicon. Said potexviral replicon further may have sub-genomic promoters in segments of item (ii) and/or (iii) for generating sub-genomic RNAs for the expression of proteins encoded by the segments of items (ii) and (iii). If said potexviral vector or the nucleic acid is DNA, it will typically have a transcription promoter at its 5′-end for allowing production by transcription of said potexviral replicon in plant cells. An example of a transcription promoter allowing transcription of said RNA replicon from a DNA nucleic acid in planta is the 35S promoter that is widely used in plant biotechnology. The 35S promoter is an example of a constitutive promoter. Constitutive transcription promoters are preferably used in the potexviral vector, notably where the potexviral vector is used for transient transfection and transient expression on the protein of interest in a plant or in plant cells. If the potexviral vector is stably integrated in chromosomal DNA of a plant or in cells of a plant, the transcription promoter may be a regulated promoter such that formation of the potexviral replicon and expression of the protein of interest ca be started at a desired point in time. An example of regulated promoters is the ethanol-inducible promoter described, for example, in WO 2007/137788 A1.

Segment (i) encodes a potexviral RdRp. The encoded potexviral RdRp may be the RdRp of a potexvirus, such as potato virus X, or it may be a function-conservative variant of an RdRp of a potexvirus. Thus, the term “potexviral” is not restricted to sequences that are exactly present in a potexvirus; the terms “potexvirus” or “of a potexvirus” mean that the designated element or segment is taken from a potexvirus. The RdRp may be considered a function-conservative variant of the RdRp of a potexvirus if said sequence of segment (i) encodes a protein having a sequence identity of at least 36% to a protein encoded by SEQ ID NO: 37. In another embodiment, said sequence identity is at least 45%, in a further embodiment at least 55%, in another embodiment at least 65% and in an even further embodiment at least 75% to a protein encoded by SEQ ID NO: 37. These sequence identities may be present over the entire sequence of SEQ ID NO: 37. Alternatively, these sequence identities may be present within a protein sequence segment of at least 300 amino acid residues, within a protein sequence segment of at least 500 amino acid residues, within a protein sequence segment of at least 900 amino acid residues, or within a protein sequence segment of at least 1400 amino acid residues.

Herein, the determination of sequence identities and similarities is done using Align Sequences Protein BLAST (BLASTP 2.6.1+) (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402).

In one example, said sequence identity between an RdRP encoded by SEQ ID NO: 37 and a function-conservative variant of a potexvirus RdRp is at least 45% in a protein sequence segment of at least 900 amino acid residues. In another example, said sequence identity between a protein encoded by SEQ ID NO: 37 and a function-conservative variant of a potexvirus RdRp is at least 55% in a protein sequence segment of at least 900 amino acid residues.

Alternatively, the RdRp used in the potexviral replicon may be considered a function-conservative variant of a RdRp of a potexvirus if said sequence of item (i) encodes a protein having a sequence similarity of at least 50% to a protein encoded by SEQ ID NO: 37. In another embodiment, said sequence similarity is at least 60%, in a further embodiment at least 70%, and in another embodiment at least 80% to a protein encoded by SEQ ID NO: 37. These sequence similarities may be present over the entire sequence of SEQ ID NO: 37. Alternatively, these sequence similarities may be present within a protein sequence segment of at least 300 amino acid residues, at least 500 amino acid residues, at least 900 amino acid residues, or at least 1400 amino acid residues. Amino acid sequence similarities may be determined using BLASTX defined above.

In one example, the sequence similarity between a protein encoded by SEQ ID NO: 37 and a function-conservative variant of a potexvirus RdRp is at least 70% in a protein sequence segment of at least 900 amino acid residues. In another example, said sequence similarity between a protein encoded by SEQ ID NO: 37 and a function-conservative variant of a potexvirus RdRp is at least 80% in a protein sequence segment of at least 900 amino acid residues.

Alternatively, the RdRp used in said potexviral replicon may be considered a function-conservative variant of a RdRp of a potexvirus if said sequence of item (i) has a sequence identity of at least 55%, of at least 60%, or of at least 70% to SEQ ID NO: 37. Said sequence identities may be present within SEQ ID NO: 37, or within a sequence segment of at least 900 nucleotides, within a sequence segment of at least 1500 nucleotides, within a sequence segment of at least 2000 nucleotides, or within a sequence segment of at least 4200 nucleotides of SEQ ID NO: 37. Nucleotide sequence identities may be determined using the BLAST given above.

The potexviral replicon comprises the nucleic acid segment of item (ii) for allowing cell-to-cell movement of said potexviral replicon in a plant or in plant tissue. Cell-to-cell movement of the potexviral replicon is important for achieving expression of the segment of item (iii) in as many cells of said plant or said tissue as possible. The nucleic acid sequence of item (ii) comprises or encodes a potexviral triple gene block (abbreviated “TGB” herein; a review on the TGB is found in J. Gen. Virol. (2003) 84, 1351-1366). The potexviral triple gene block encodes three proteins necessary to provide the capability of cell-to-cell movement to a potexvirus. The term “potexviral triple gene block” includes variants of the TGB of a potexvirus, provided the variants can provide, optionally with other necessary components, the potexviral replicon of the invention with the capability of cell-to-cell movement in a plant or in plant tissue.

Examples of a potexviral TGB are TGBs of a potexvirus. An example of a potexviral TGB is the TGB of potato virus X (referred to as “PVX TGB” herein). The PVX TGB consists of three genes encoding three proteins designated 25K, 12K, and 8K according to their approximate molecular weight. The gene sequences encoding the PVX 25K, the PVX 12 K protein, and the PVX 8K protein are given in SEQ ID NO: 29, SEQ ID NO: 31, and SEQ ID NO: 33, respectively. Protein sequences of the PVX 25 K protein, the PVX 12K protein, and the PVX 8K protein are given in SEQ ID NO: 30, SEQ ID NO: 32, and SEQ ID NO: 34, respectively.

In one embodiment, said variant of a potexvirus TGB is a block of three genes, said block encoding three proteins one of which having a sequence identity of at least 33% to the PVX 25K protein, one having a sequence identity of at least 36% to the PVX 12K protein and one having a sequence identity of at least 30% to the PVX 8K protein. In another embodiment, said function-conservative variant of a potexvirus TGB encodes three proteins one of which having a sequence identity of at least 40% to the PVX 25K protein, one having a sequence identity of at least 40% to the PVX 12K protein, and one having a sequence identity of at least 40% to the PVX 8K protein. In a further embodiment, said function-conservative variant of a potexvirus TGB encodes three proteins one of which having a sequence identity of at least 50% to the PVX 25K protein, one having a sequence identity of at least 50% to the PVX 12K protein and one having a sequence identity of at least 50% to the PVX 8K protein. In a further embodiment, the corresponding sequence identity values are at least 60% for each protein. In a further embodiment, the corresponding sequence identity values are at least 70%, preferably at least 80%, for each protein.

In another embodiment, a function-conservative variant of a potexvirus TGB encodes three proteins as follows: a first protein comprising a protein sequence segment of at least 200 amino acid residues, said segment having a sequence identity of at least 40% to a sequence segment of the PVX 25K protein; a second protein comprising a protein sequence segment of at least 100 amino acid residues, said sequence segment having a sequence identity of at least 40% to a sequence segment of the PVX 12K protein; and a third protein comprising a protein sequence segment of at least 55 amino acid residues, said sequence segment having a sequence identity of at least 40% to a sequence segment of the PVX 8K protein. In a further embodiment, the corresponding sequence identity values are at least 50% for each protein. In a further embodiment, the corresponding sequence identity values are at least 60% for each of said first, second, and third protein.

Said nucleic acid sequence of item (ii) preferably comprises a further sequence encoding a protein for cell-to-cell movement and long distance movement of said potexviral replicon such as a potexvirus coat protein or a function-conservative variant thereof. A variant of said potexvirus coat protein is considered a function-conservative variant of said coat protein if it is capable of providing said potexviral replicon, together with other necessary components such as the TGB, with the capability of cell-to-cell movement and long distance movement in a plant or in plant tissue. In one embodiment where said potexviral replicon comprises a potexviral coat protein, said potexviral replicon does not have an origin of viral particle assembly for avoiding spread of said potexviral replicon from plant to plant in the form of an assembled plant virus. If said potexviral replicon comprises a potexviral coat protein gene and a potexviral TGB, it is possible that said TGB is located upstream of said coat protein gene or vice versa. Thus, said potexviral coat protein gene and said potexviral TGB may be present in any order in said nucleic acid sequence of item (ii).

The coding sequence of a PVX coat protein is given as SEQ ID NO: 35, and the amino acid sequence of the PVX coat protein is given as SEQ ID NO: 36. A protein can be considered a function-conservative variant of a potexvirus coat protein if it comprises a protein sequence segment of at least 200, alternatively at least 220, further alternatively 237 amino acid residues, said sequence segment having a sequence identity of at least 35% to a sequence segment of SEQ ID NO: 36. In another embodiment, a protein is considered a function-conservative variant of a potexvirus coat protein if it comprises a protein sequence segment of at least 200, alternatively at least 220, further alternatively 237 amino acid residues, said sequence segment having a sequence identity of at least 45% to a sequence segment of SEQ ID NO: 36. In alternative embodiments, the corresponding sequence identity values are at least 55%, preferably at least 65%, and more preferably at least 75%.

Alternatively, said nucleic acid sequence of item (ii) may comprise, optionally instead of said sequence encoding said potexviral coat protein or variant thereof, a sequence encoding a plant viral movement protein (MP). An example of a suitable MP is a tobamoviral MP such as an MP of tobacco mosaic virus or an MP of turnip vein clearing virus. Said sequence encoding a plant viral movement protein and said potexvirus TGB (or a function-conservative variant thereof) may be present in any order in said nucleic acid sequence of item (ii).

As described above, the heterologous nucleic acid sequence of item (iii) comprises at least the ORF of a protein of interest to be expressed in a plant or in plant tissue. The heterologous nucleic acid sequence of item (iii) corresponds to the second heterologous nucleic acid sequence of the method claims. Said heterologous sequences are heterologous in that they are heterologous to the potexvirus on which said potexviral replicon is based. In many cases, said sequences are also heterologous to said plant or said plant tissue in which it is to be expressed. For being expressible from said potexviral replicon in a plant or in plant tissue, the second heterologous nucleic acid of item (iii) typically comprises a sub-genomic promoter and other sequences required for expression such as ribosome binding site and/or an internal ribosome entry site (IRES). In a preferred embodiment, the second heterologous nucleic acid of item (iii) has one ORF that codes for one protein of interest. The protein of interest of the invention is preferably not a plant viral protein or it is a protein that is heterologous to plant viruses, notably it should be heterologous to the potexvirus on which said potexviral replicon is based. A plant viral protein is a protein encoded by a plant virus. I one embodiment, said protein of interest is neither a potexviral coat protein nor a tobamoviral movement protein.

The nucleic acid, the potexviral vector and/or the potexviral replicon of the invention may comprise a potexviral or, preferably, a potexvirus 5′-nontranslated region (5′-NTR) and a potexviral or, preferably, a potexvirus 3′-nontranslated region (3′-NTR).

Preferred methods of the invention are as follows:

a method of improving the capability for long-distance movement in a plant of a potexviral replicon encoding a protein of interest to be expressed in said plant, comprising

producing a second heterologous nucleic acid sequence comprising a second ORF encoding said protein of interest and having, in the second ORF, an increased GC-content compared to a first ORF encoding said protein of interest in a first heterologous nucleic acid sequence, and

providing said potexviral replicon, or a potexviral vector comprising or encoding said potexviral replicon, said potexviral replicon comprising the following segments: (i) a nucleic acid sequence encoding a potexviral RNA-dependent RNA polymerase, (ii) a nucleic acid sequence comprising (a) a potexviral triple-gene block and (b) a nucleic acid sequence encoding a potexviral coat protein or a nucleic acid sequence encoding a tobamoviral movement protein, and (iii) said second ORF, said second heterologous nucleic acid sequence or a portion of the latter that comprises said second ORF;

a method of improving the capability for long-distance movement in a plant of a potexviral replicon encoding a protein of interest to be expressed in said plant, comprising producing a second heterologous nucleic acid sequence comprising a second ORF encoding said protein of interest and having, in the second ORF, an increased GC-content compared to a first ORF encoding said protein of interest in a first heterologous nucleic acid sequence, and

providing said potexviral replicon, or a potexviral vector comprising or encoding said potexviral replicon, said potexviral replicon comprising the following segments: (i) a nucleic acid sequence encoding a potexviral RNA-dependent RNA polymerase, (ii) a nucleic acid sequence comprising (a) a potexviral triple-gene block and (b) a nucleic acid sequence encoding a potexviral coat protein or a nucleic acid sequence encoding a tobamoviral movement protein, and (iii) said second ORF, said second heterologous nucleic acid sequence or a portion of the latter that comprises said second ORF.

In these preferred embodiments, the order of the segments (i) to (iii) is preferably in this order from the 5′-end to the 3′-end of the vector or replicon. Alternatively, the order may be segments (i), (iii), and (ii) from the 5′-end to the 3′-end of the vector or replicon. In segment (ii), the order of sub-items (ii-a) and (ii-b) is not limited. However, the order may be from (ii-a) to (ii-b) in the 5′- to 3′-direction of the vector or replicon. The nucleic acid sequence encoding a potexviral coat protein and a nucleic acid sequence encoding a tobamoviral movement protein are as described elsewhere herein.

The process of expressing a protein of interest in a plant or in plant tissue of the invention generally comprises providing a plant or plant tissue with said nucleic acid or potexviral vector of the invention. It is of course also possible to infect a plant or plant tissue with the potexviral replicon of the invention. In one embodiment, said process is a transient expression process, whereby incorporation of the nucleic acid or potexviral vector of the invention into chromosomal DNA of the plant host is not necessary and not selected for. Alternatively, the potexviral vector may be stably incorporated into chromosomal DNA to produce a transgenic plant. The production of transgenic plants is known to the skilled person and comprises, inter alia, transformation of plant cells or tissue, selection of transformed cells or tissue, and regeneration of transformed plants.

If said nucleic acid or said potexviral vector of the invention is RNA, it may be used for infecting a plant or plant tissue, preferably in combination with mechanical injury of infected plant tissue such as leaves. In another embodiment, said nucleic acid or potexviral vector of the invention is DNA. Said DNA may be introduced into cells of a plant or plant tissue, e.g. by particle bombardment or by Agrobacterium-mediated transformation. Agrobacterium-mediated transformation is the method of choice if several plants are to be provided with said nucleic acid or potexviral vector of the invention, e.g. for large scale protein production methods. Particularly efficient methods for Agrobacterium-mediated transformation or transfection are described in WO 2012/019660 and WO 2013/056829.

The process of expressing a protein of interest in a plant may be performed using the pro-vector approach (described in W002088369 and by Marillonnet et al., 2004, Proc. Natl. Acad. Sci. USA, 101:6852-6857) by providing a plant or plant tissue with said kit or combination of nucleic acids of the invention. In this embodiment, the nucleic acid of the invention is produced by site-specific recombination between a first and a second nucleic acid in cells of said plant. Said first and a second nucleic acid act as the pro-vectors described in WO02088369 and by Marillonnet et al. (above) and are also referred to herein as pro-vectors. In one embodiment, a first nucleic acid (pro-vector) comprising or encoding segments of items (i) and (ii) and a second nucleic acid (pro-vector) comprising or encoding the segment of item (iii) is provided to a plant or plant tissue (e.g. by Agrobacterium-mediated transformation such as infiltration), wherein said first and said second pro-vector each has a recombination site for allowing assembly of a nucleic acid of the invention by site-specific recombination between said first and said second pro-vector. Preferable, said first nucleic acid has, downstream of segment (ii) a first site-specific recombination site recognizable by a site-specific recombinase, and said second nucleic acid has, upstream of segment (iii), a second site-specific recombination site recognizable by a, preferably the same, site-specific recombinase for allowing site-specific recombination between said first and said second site-specific recombination site and formation of a nucleic acid according to the invention.

Two or more vectors or said first and second nucleic acids may be provided to a plant or to plant tissue by providing mixtures of the vectors or mixtures of Agrobacterium strains, each strain containing one of said vectors or pro-vectors, to a plant or to plant tissue. The plant or plant tissue may further have or be provided with a site-specific recombinase recognizing the recombination sites of the first and second nucleic acids (pro-vectors). If the plant or plant tissue does not express the recombinase, a plant-expressible gene encoding the recombinase may be provided to the plant or plant tissue on one of said pro-vectors or on a separate vector. Examples of a usable site-specific recombinase are as described in WO02088369; an integrase as mentioned therein is also considered a site-specific recombinase.

Said protein of interest may be purified after production in said plant or plant tissue. Methods or purifying proteins from plants or plant cells are known in the art. In one method, a protein of interest may be directed to a plant apoplast and purified therefrom as described in WO 03/020938.

If one protein of interest has to be produced or expressed, a heterologous nucleic acid or ORF coding for said protein of interest may be included in said nucleic acid encoding said potexviral replicon. If two or more proteins of interest are to be produced in the same plant or in the same plant tissue, said plant or plant cells may be provided with another nucleic acid or potexviral vector comprising or encoding a further potexviral replicon. Said further potexviral replicon may then encode one or more further proteins of interest. In one embodiment, a first and a further nucleic acid of the invention may comprise or encode non-competing potexviral replicons as described in WO 2006/079546.

The process of expressing a protein of interest in a plant of the present invention is, with regard to the plant, not particularly limited. In one embodiment, dicotyledonous plants or tissue thereof are used. In another embodiment, Nicotiana species like Nicotiana benthamiana and Nicotiana tabacum are used; preferred plant species other than Nicotiana species are tomato, potato, pepper, eggplant, soybean, Petunia hybrida, Brassica napus, Brassica campestris, Brassica juncea, cress, arugula, mustard, strawberry, spinach, Chenopodium capitatum, alfalfa, lettuce, sunflower, potato, cucumber, corn, wheat and rice.

The most preferred plant viruses the potexviral replicons of the invention may be based on are Potexviruses such as potato virus X (PVX), papaya mosaic potexvirus or bamboo mosaic potexvirus.

The invention may also be used for improving the capability for long-distance movement in a plant of a potexviral RNA replicon encoding a protein to be expressed in said plant. In one embodiment, the method comprises the following steps:

a step of increasing the GC-content of a first ORF encoding said protein in a first heterologous nucleic acid sequence, thereby obtaining a second heterologous nucleic acid sequence comprising a second ORF, said second ORF encoding said protein and having an increased GC-content, and

a step of inserting said second heterologous nucleic acid sequence, or a portion thereof containing said second ORF, into a nucleic acid comprising (i) a nucleic acid sequence encoding a potexviral RNA-dependent RNA polymerase and (ii) a nucleic acid comprising or encoding a potexviral triple-gene block to produce a potexviral vector comprising or encoding said RNA replicon, said potexviral vector comprising the second heterologous nucleic acid or a portion thereof comprising said second ORF.

In another embodiment, the method comprises the following steps:

a step of producing a second heterologous nucleic acid sequence comprising a second ORF encoding said protein and having, in the second ORF, an increased GC-content compared to a first ORF encoding said protein in a first heterologous nucleic acid sequence, and

a step of providing said potexviral RNA replicon, or a potexviral vector comprising or encoding said potexviral RNA replicon, said potexviral RNA replicon comprising the following segments: (i) a nucleic acid sequence encoding a potexviral RNA-dependent RNA polymerase, (ii) a nucleic acid sequence comprising a potexviral triple-gene block, and (iii) said second heterologous nucleic acid or a portion thereof comprising said second ORF.

The above increasing, inserting, producing and providing steps may be performed similarly as described above. The methods of increasing the capability for long-distance movement in a plant of a potexviral replicon may be followed by providing the obtained potexviral replicon or potexviral vector to at least a part of said plant. An increase of the capability for long-distance movement in a plant may be followed experimentally, e.g. as described in the Examples. Generally, a plant may be provided with the potexviral vector on a selected leaf. After a predetermined period of time, e.g. after 5 days, after 7 days or after 9 days, tissue of systemic leaves may be investigated for the presence of the potexviral replicon encoded by the potexviral vector. RT-PCR may be used for testing any potexviral replicon in a systemic leaf for correctness and/or presence of all components of the potexviral replicon encoded by the potexviral vector. A systemic leaf is a leaf other than an inoculated leaf; a systemic leaf is a leaf where virus moves from a site of primary infection or transfection in inoculated leaf due to a long-distance systemic movement.

### EXAMPLES

### Example 1: Plasmid Constructs

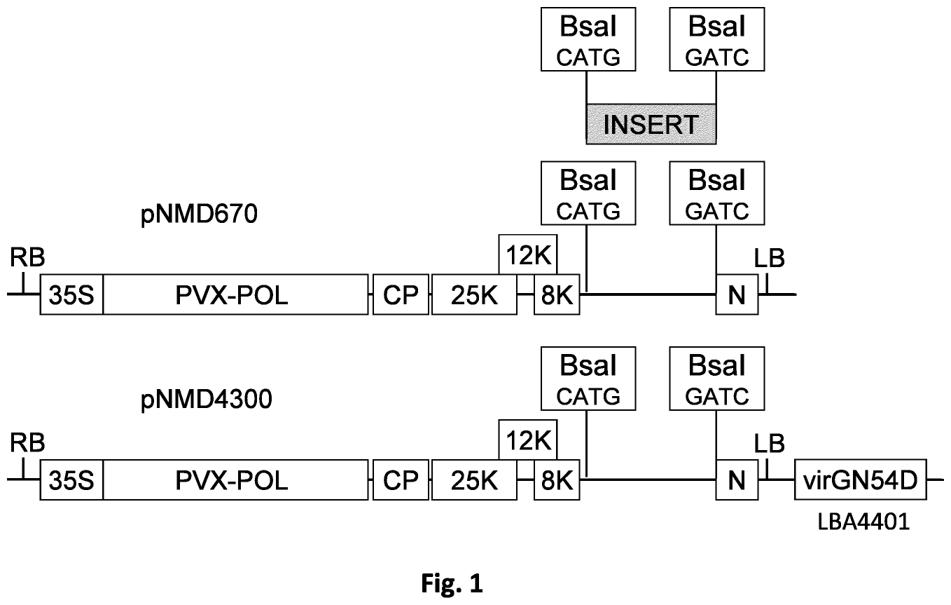

PVX-based assembled viral vectors pNMD670 and pNMD4300 (FIG. 1) were used for cloning of DNA inserts of interest. pNMD4300 is a modified version of pNMD670 construct which is described in WO 2012/019660. In contrast to pNMD670, pNMD4300 contains virG N54D mutant gene sequence from LBA4404 strain of Agrobacterium tumefaciens (GenBank Accession No CP007228, nucleotide positions 161000-161725) inserted into the plasmid backbone for increasing the efficiency of T-DNA transfer.

Nucleotide sequences of inserts of interest were either directly retrieved from GenBank or designed with modified GC content based on codon usage optimized for certain organisms. Sequences for cloning were either amplified using cDNA as a template or synthesized by Eurofins Genomics (Eurofins Genomics GmbH, Ebersberg, Germany). Codon usage modification was performed with Eurofins Genomics online tool based on codon usage patterns of organisms differing in average GC content (GENEius software). Inserts of interest were subcloned into pNMD4300 vector using Bsal restriction sites with CATG and GATC overhangs (FIG. 1). Flanking Bsal sites were added to sequences of interest either by PCR or during gene synthesis.

Sequences of gene inserts used for cloning are listed in Table1, Table 2 and Table 3.

### Example 2: PVX Vector Stability with Inserts Differing in Length, GC Content and Ratio Between GC Content and the Length

We subcloned AtFT, CaDREB-LP1, AmROS1, GmLOG1, SILOG1, sGFP, SIGR, SIDREB1, SIOVATE, SISUN, GUS and SIWoolly coding sequences (12 in total) into pNMD4300 cloning vector (Table 1). All of them except sGFP were native sequences from corresponding organisms. sGFP is a synthetic coding sequence for Green Fluorescent Protein from a jellyfish Aequorea victoria altered to conform to the favored codons of highly expressed human proteins which resulted in a substantial increase in expression efficiency (Haas et al 1996; Chiu et al 1996).

Gene inserts differed in their Length and GC content. The shortest insertion was AtFT (528b), and the longest one was SIWoolly (2193 bp) (Table 1). The GC content of inserts was determined using ENDMEMO on-line DNA/RNA GC Content Calculator (www.endmemo.com/bio/gc.php). The GC content of listed inserts was in in the range between 40.0% (SISUN) and 61.4% (sGFP) (Table 1). We also calculated the Ratio between GC content and Length of inserts. It was done using the following formula:

Ratio GC Content/Length=(GC content (%)/Length (bp)×100.

Multiplier x100 was used for convenience to avoid too small fractional numbers. According to this formula, the Ratio GC Content/Length varied between 2.0 (SIWoolly) and 8.6 (AtFT).

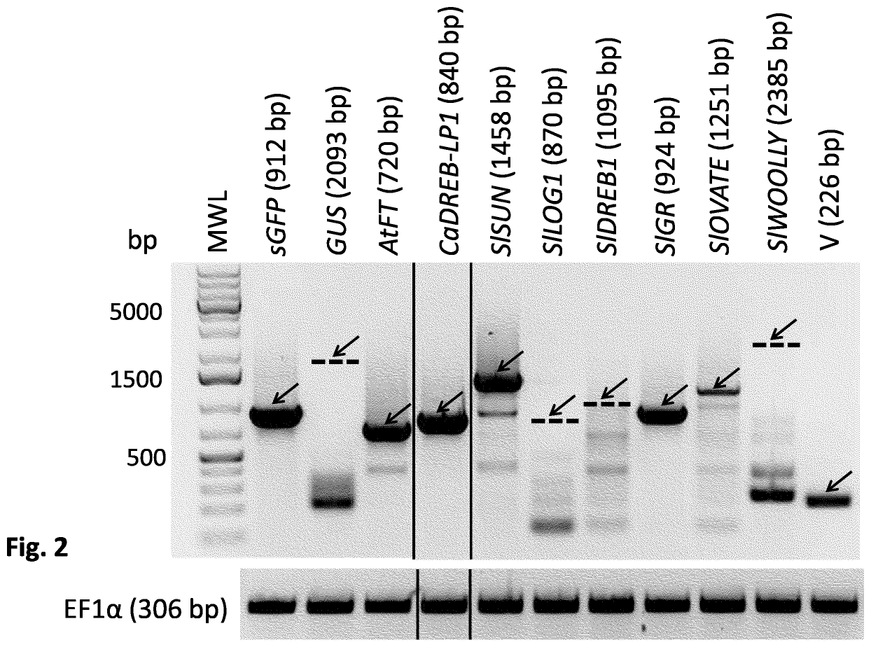

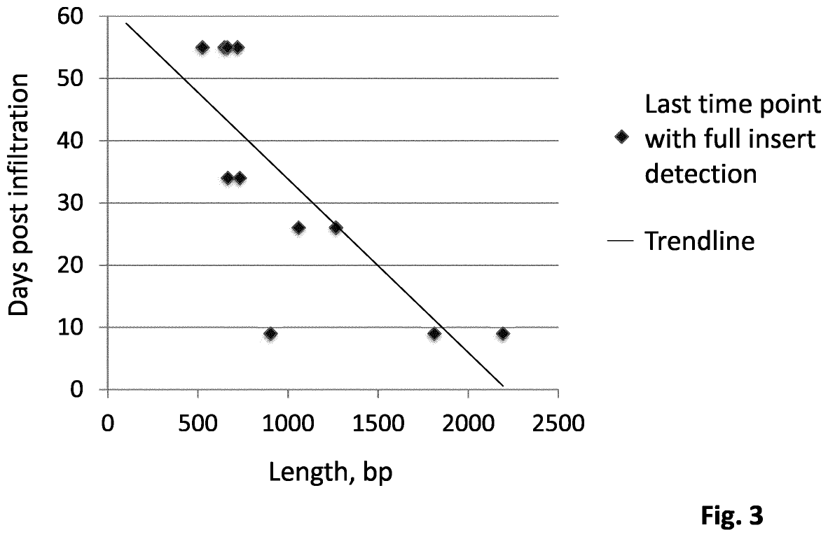

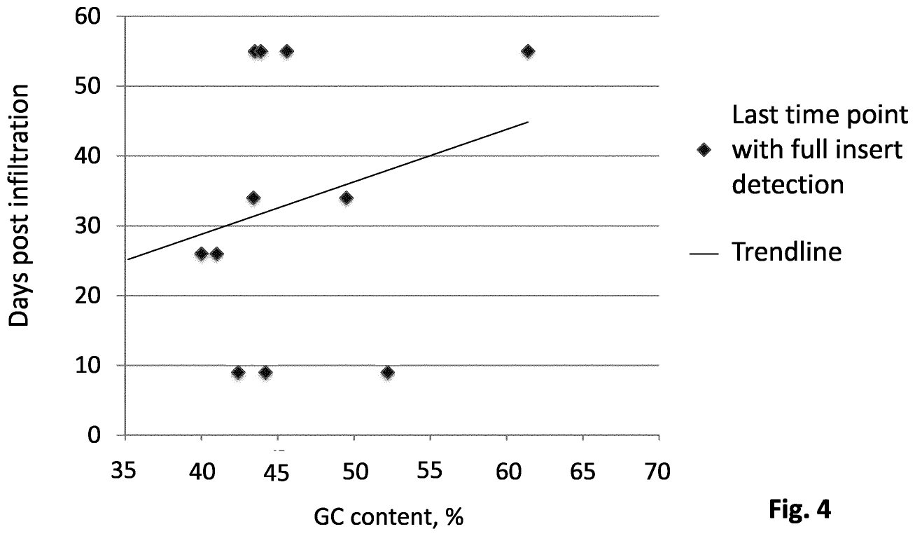

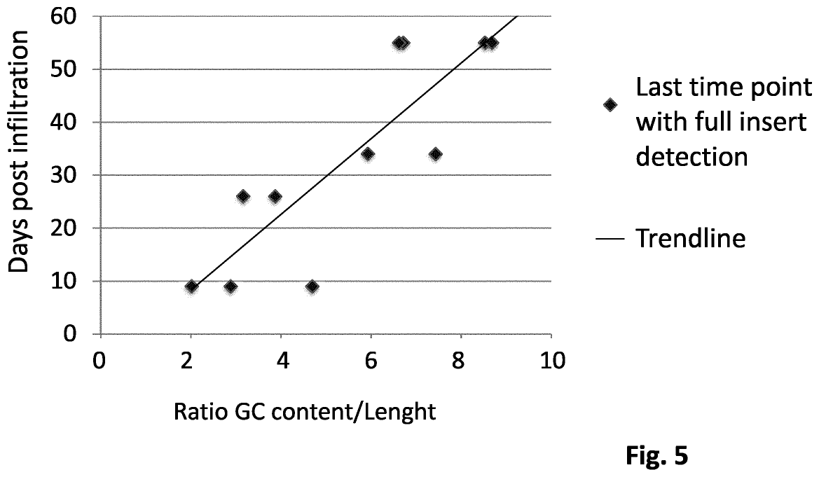

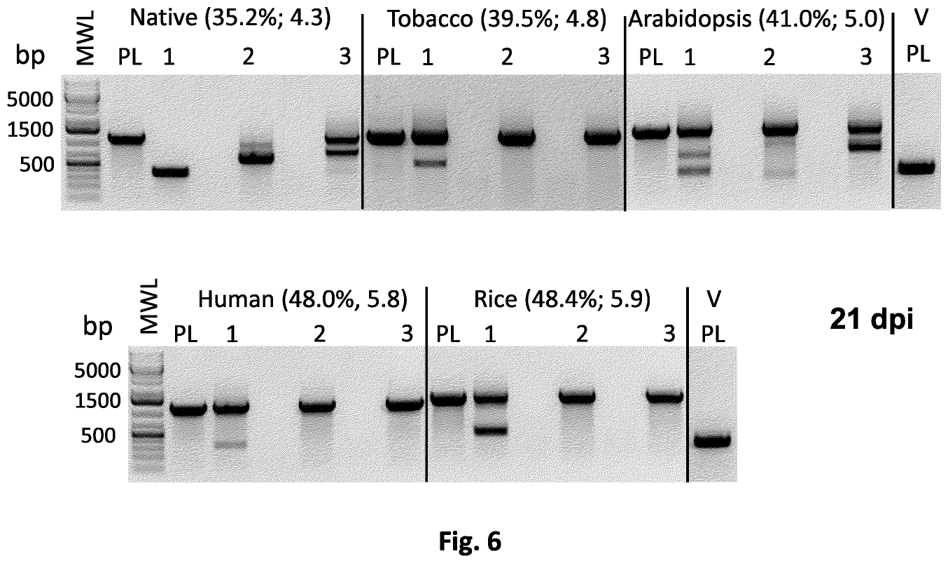

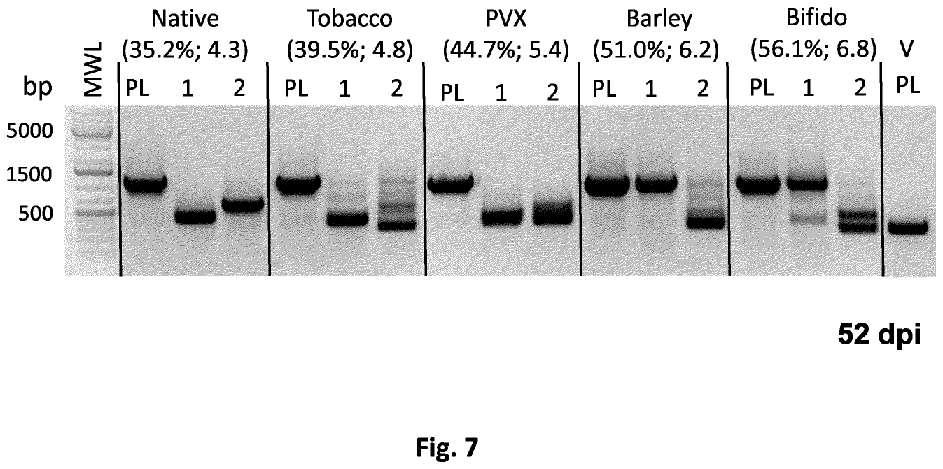

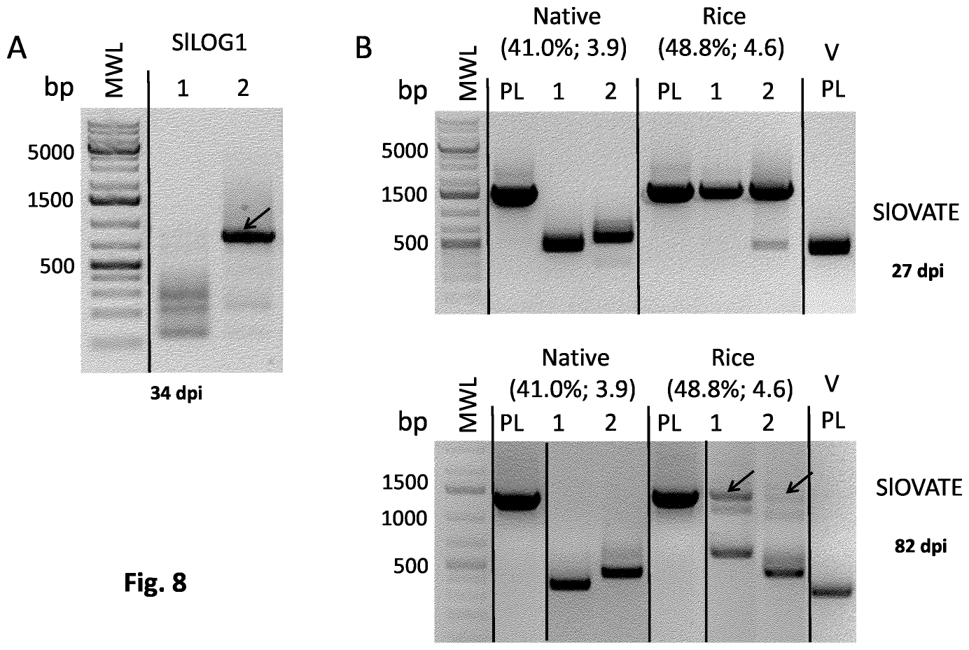

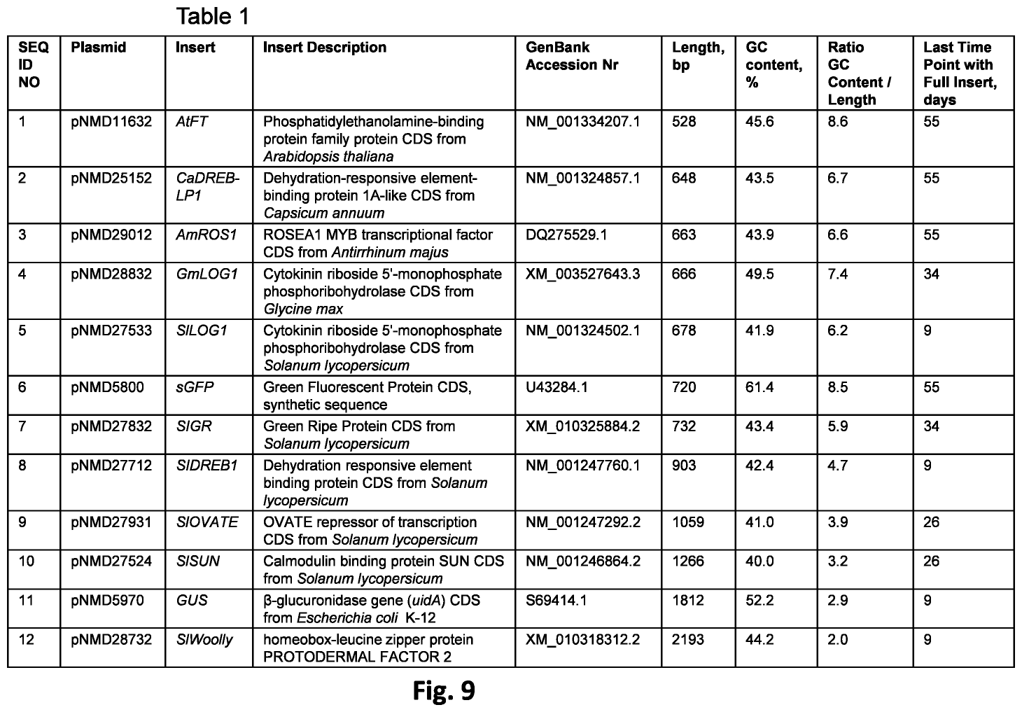

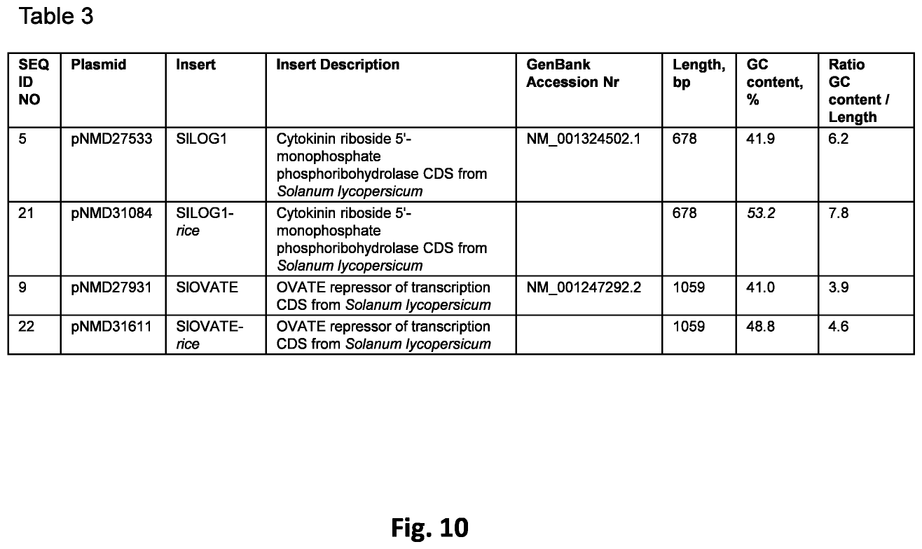

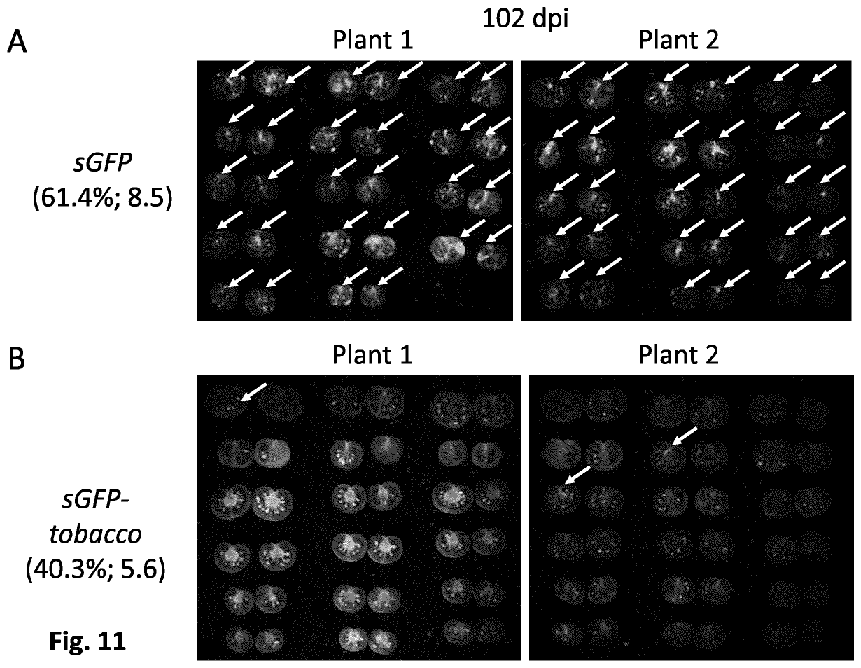

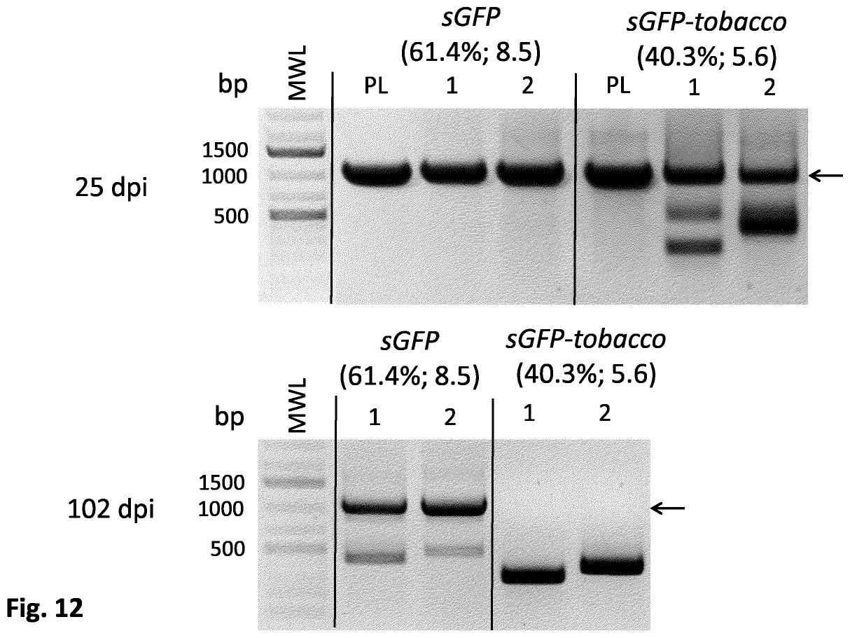

Cotyledons and the two first true leaves of 36 days old tomato Solanum lycopersicum ‘Balcony Red’ plants were syringe inoculated with agrobacterial cultures carrying PVX vectors listed in Table 1 (one independent plant per one construct). Plant material from infiltrated leaves was harvested using the cork borer at 9 dpi; the material from systemic leaves was harvested at 26, 27, 34 and 55 dpi. Total RNA isolated from harvested plant material reverse transcribed using PrimeScript™ RT Reagent Kit (Takara Clontech) and oligo dT primer. Resulting cDNA was used as a template for PCR with oligos specific for either PVX (8K-RT: tttgaagacatctcaacgcaatcatacttgtgc (SEQ ID NO: 25) and 3NTR-RT: tttgaagacttctcggttatgtagacgtagttatggtg (SEQ ID NO: 26)) or Elongation Factor EF1a from N. benthamiana (Genbank No. AY206004.1, oligos NbEF_for and NbEF_rev (Dean et al., 2005) used as an RNA loading control. PCR products were resolved in 1% agarose gel. FIG. 2 illustrates the result of RT-PCR analysis for PVX vectors containing insertions of sGFP, GUS, AtFT, CaDREB-LP1, SISUN, SILOG1, SIDREB1, SIGR, SIOVATE and SIWoolly genes 26 days post infiltration. As one can see, at this time point PVX vectors with sGFP, AtFT, CaDREB1-LP1, SISUN and SIGR remain pretty stable. In contrast, vectors with GUS, SILOG1, SIDREB1 and SIWoolly have already lost their inserts.

The last day post infiltration when full-length insert was detected (even if additional shorter fragments resulting from partial insert elimination were present) was considered as a Last Time Point with Full Insert and used as criterion of vector stability (Table 1). We found the vectors with AtFT, CaDREB-LP1, AmROS1, and sGFP to be most stable: their inserts were detectable at 55 dpi. Vectors with SILOG1, SIDREB1, GUS and SIWoolly genes were highly unstable: their inserts were not detectable in systemic leaves at all. Vectors with GmLOG1, SIGR, SIOVATE and SISUN had moderate stability: their inserts were lost after 26-34 dpi.

We analyzed the relation between Length of tested inserts and their Stability. For this purpose, we plotted the Last Time Point with Full Insert (Y-axis) against the Length of Insert (X axis) (FIG. 3). We found that increasing the size of insert results in decreased vector stability, which is in accordance with former data from literature (e. g. Avesani et al., 2007).

We also analyzed the relation between GC content and Stability of inserts (FIG. 4). We did not find a clear trend for analyzed pool of sequences probably due to large difference in size between individual inserts (e.g. 4 times difference between SIWoolly and AtFT). We then analyzed the relation between the Ratio GC Content/Length and Insert Stability. In this case, a clear trend was observed: an increase of the Ratio GC Content/Length resulted in an increase of insert stability (FIG. 5).

### Example 3: Improving the Stability of PVX with SIANT1 Insert

Solanum lycopersicum anthocyanin 1 (SIANT1) gene (AY348870.1) codes for MYB transcription factor anthocyanin 1 (SIANT1) (AAQ55181). ANT1 transcriptional factor activates the biosynthetic pathway leading to anthocyanin accumulation; plants overexpressing ANT1 gene acquire intensive purple coloration due to anthocyanin accumulation (Mathews et al 2003).

We tried to overexpress SIANT1 gene in tomato ‘Balcony Red’ plants using PVX-based viral vector. Native SIANT1 coding sequence was subcloned into pNMD670 (without VirG) vector resulting in pNMD721 construct (Table 2). The pNMD721 construct was tested in planta using agrobacterial delivery via syringe infiltration of 28 days old plants. 21 dpi, relatively dense purple coloration was observed in infiltrated leaves. In contrast, few sparse colored spots were observed in systemic leaves. We analyzed systemic leaves of 3 independent plants transfected with this vector for the integrity of SIANT1 insert. RT-PCR analysis was performed as described in Example 2. It detected the loss of the insert by the PVX vector.

TABLE 2
SIANT1 sequences with different codon usage (Example 3).

| | | | | | Ratio |
|---|---|---|---|---|---|
| SEQ | | | | GC | GC |
| ID | | | Length, | content, | content/ |
| NO: | Plasmid | Codon usage | bp | % | Length |
| 13 | pNMD721 | Solanum | 825 | 35.2 | 4.3 |
| | | lycopersicum, | | | |
| | | native (GenBank | | | |
| | | Accession No. | | | |
| | | AY348870.1) | | | |
| 14 | pNMD29561 | Nicotiana tabacum | 825 | 39.5 | 4.8 |
| 15 | pNMD29541 | Arabidopsis thaliana | 825 | 41.0 | 5.0 |
| 16 | pNMD30881 | Potato Virus X | 825 | 44.7 | 5.4 |
| 17 | pNMD29531 | Homo sapiens | 825 | 48.0 | 5.8 |
| 18 | pNMD29551 | Oryza sativa | 825 | 48.4 | 5.9 |
| 19 | pNMD30722 | Hordeum vulgare | 825 | 51.0 | 6.2 |
| 20 | pNMD30891 | Bifidobacterium | 825 | 56.1 | 6.8 |

SIANTI1 sequence analysis revealed very low GC content (35.2%) and quite low Ratio GC content/Length (4.3). We designed 7 new sequence versions with increased GC content and, as result, Ratio between GC content and Length (Table 2). The design was performed using online codon optimization tool from Eurofins Genomics (GENEius software) based on codon usage of organisms with different average values of GC content in their genomes (data retrieved from Kazusa Codon Usage Database (www.kazusa.or.jp/codon/)). For this purpose, we selected codon usage patterns of Nicotiana tabacum, Arabidopsis thaliana, Potato Virus X, Homo sapiens, Oryza sativa, Hordeum vulgare and Bifidobacterium with average GC content 39.2%, 41.0%, 44.7%, 48.0%, 48.4%, 51.0%, and 56.1%, respectively. Additionally, poly dA (AAAAA and AAAAAAA) and poly dT (TTTTT) sequences as well as Bsal cleavage sites (GGTCTCNNNNN (SEQ ID NO: 27)) and predicted donor/acceptor splicing sites (AGGTRAG/GCAGGT (SEQ ID NO: 28)) were avoided inside sequences. Designed sequences were synthesized by Eurofins Genomics and subcloned into pNMD670 vector resulting in constructs listed in Table 2. All constructs were tested in tomato ‘Balcony Red’ using agrobacterial delivery via syringe infiltration (3 independent 28 days old plants per one construct). Systemic leaves of infected tomato plants were analyzed for PVX vector integrity at 21 and 52 dpi (FIGS. 6 and 7).

At 21 dpi, complete loss of the insert with native sequence was found in 2 out of 3 plants. In one plant both intact and partially degraded vector sequences were detected (FIG. 6). For all other sequences (codon optimization for tobacco, Arabidopsis, human and rice), all tested plants contained intact vector sequence, although in some cases additional bands indicating partial loss of the insertion were also present (FIG. 6).

At 52 dpi, 2 plants for each construct were analyzed (FIG. 7). We found complete loss of the insert for native sequence in both plants. Vector degradation was also observed for tobacco and PVX-optimized sequences with lower GC content and Ratio between GC content and Length. In contrast, for sequences with higher GC content (barley and Bifidobacterium codon usage) one of two plants contained intact vectors with SIANT1 insertion (FIG. 7).

These data show that increasing the GC content of the foreign insert sequence and, correspondingly, the ratio between the GC content and Length allows improving the stability and increasing the lifetime of systemic PVX vector.

### Example 4: Improving the Stability of PVX with SILOG1 and SIOVATE Inserts

We also tried to improve the stability of PVX vectors with SILOG1 and SIOVATE inserts. As it was shown in Example 2, SILOG1 insert with native sequence (pNMD27533) was not detectable in systemic leaves, indicating very high instability (Table 1). SIOVATE (pNMD27931) showed moderate stability; intact insert as well as products of degradation was still detectable in systemic leaves at 26 dpi; however, the intact insert was completely lost already at 27 dpi (Table 1).

SILOG1 native sequence is 678 bp in length; it has 41.9% GC content and 6.2 ratio between GC content and Length. SIOVATE is 1059 bp long; it has it has 41.0% GC content and 3.9 ratio between GC content and Length. We redesigned both sequences based on rice adapted codon used. Resulting sequences had increased GC content: 53.2% for SILOG1-rice and 48.8% for SIOVATE-rice. Both sequences were synthesized by Eurofins MWG Operon and subcloned into pNMD4300 vector.

Resulting constructs (pNMD31084 for SILOG1 -rice and pNMD31611 for SIOVATE-rice) were tested in 24 and 25 days old tomato ‘Balcony Red’ plants as described in Example 2. At 34 dpi, RT-PCR analysis revealed the dramatic increase of SILOG1 -rice insert stability if compared with native sequence (FIG. 8, A). Significant increase of insert stability was also shown for codon-optimized SIOVATE. Rice codon usage adapted inserts remain intact at 27 dpi, whereas native sequence is completely lost (FIG. 8, B, Upper panel). Despite the presence of products of vector degradation, one can detect the intact insert of SIOVATE-rice (FIG. 8, B, Lower panel) even 82 dpi.

### Example 5: Decreasing the Stability of sGFP Insert in PVX Vector

We also analyzed whether decrease in GC content of the insert results in the PVX vector instability.

sGFP (SEQ ID NO: 6) has 61.4% GC content and 8.53 Ratio between GC content and Length. In our experiments, PVX vectors with sGFP insert demonstrated high degree of stability. We redesigned sGFP sequence based on Nicotiana tabacum adapted codon usage. The resulting sequence (sGFP-tobacco, SEQ ID NO: 38) had 40.3% GC content and 5.60 Ratio between GC content and Length.

sGFP and sGFP-tobacco sequences were subcloned into pNMD4300 vector, resulting in pNMD5800 and pNMD32685 constructs, respectively. Both constructs were transferred into Agrobacterium tumefaciens NMX021 cells.

First photosynthetic leaves of 25 days old tomato ‘Balcony Red’ plants were inoculated with Agrobacterium cultures carrying pNMD5800 and pNMD32685 constructs (two plants per construct). The inoculation was performed using syringe infiltration with a 1:100 dilution of agrobacterial suspension of OD600=1.5.

After 25 dpi, samples from systemic leaves of inoculated plants were taken for RT-PCR analysis.

After 102 dpi, all mature fruits of inoculated plants were collected and analyzed for GFP fluorescence using visual inspection in UV light. Fruit samples were also subjected to RT-PCR analysis. All fruits of the pNMD5800 treated plants (original sGFP sequence) showed GFP fluorescence (FIG. 11, A). In contrast, only a few fruits of two plants which were transfected with pNMD32685 construct (sGFP-tobacco sequence) showed tiny GFP spots (FIG. 11, B).

Vector insert stability was analyzed using RT-PCR. The RNA isolated from 25 dpi leaf samples and 102 dpi samples of fruits was used for cDNA synthesis. Resulting cDNA samples were used as templates for PCR amplification with PVX-specific oligos 8K-RT (tttgaagacatctcaacgcaatcatacttgtgc) (SEQ ID NO: 25) and pvx3NTR-RT (tttgaagacttctcggttatgtagacgtagttatggtg) (SEQ ID NO: 26). As it is shown in FIG. 12, the degradation of sGFP-tobacco construct was detectable in systemic leaves already after 25 dpi (upper panel). It further continued so that only one degradation product per plant could be detected after 102 dpi (lower panel). It has to be noted that the original sGFP construct with higher GC content was stable at 25 dpi (upper panel) and 102 dpi (lower panel). Some minor degradation products were detectable only at 102 dpi (lower panel).

These data clearly show that the decrease in GC content of PVX vector insert results in the decrease of vector stability.

### REFERENCES

1) Haas J., Park E. C., and Seed B. (1996) Codon usage limitation in the expression of HIV-1 envelope glycoprotein, Curr Biol 6(3): 315-24.
2) Chiu W., Niwa Y., Zeng W., Hirano T., Kobayashi H., and Sheen J. (1996) Engineered GFP as a vital reporter in plants, Curr Biol 6(3): 325-30.
3) Dean J. D., Goodwin P. H., Hsiang T. (2005) Induction of glutathione S-transferase genes of Nicotiana benthamiana following infection by Colletotrichum destructivum and C. orbiculare and involvement of one in resistance 56(416): 1525-1533.
4) Avesani L., Marconi G., Morandini F., Albertini E., Bruschetta M., Bortesi L., Pezzotti M., Porceddu A. (2007) Stability of Potato Virus X expression vectors is related to insert size: implications for replication models and risk assessment, Transgenic Res 16(5): 587-97.
5) Mathews H., Clendennen S. K., Caldwell C. G., Liu X. L., Connors K., Matheis N., Schuster D. K., Menasco D. J., Wagoner W., Lightner, J. and Wagner D. R. (2003) Activation tagging in tomato identifies a transcriptional regulator of anthocyanin biosynthesis, modification, and transport, Plant Cell 15 (8), 1689-1703.

Nucleotide and amino acid sequences

SEQ ID NO: 1
AtFT (NM_001334207.1)/one nucleotide exchange (deletion of BsaI-cleavage site)
Atgtctataaatataagggaccctcttatagtaagcagagttgttggagacgttcttgatccgtttaatagatcaatcactctaaag
gttacttatggccaaagagaggtgactaatggcttggatctaaggccttctcaggttcaaaacaagccaagagttgagattggtgga
gaagacctcaggaacttctatactttggttatggtggatccagatgttccaagtcctagcaaccctcacctccgagaatatctccat
tggttggtgactgatatccctgctacaactggaacaacctttggcaatgagattgtgtgttacgaaaatccaagtcccactgcagga
attcatcgtgtcgtgtttatattgtttcgacagcttggcaggcaaacagtgtatgcaccagggtggcgccagaacttcaacactcgc
gagtttgctgagatctacaatctcggccttcccgtggccgcagttttctacaattgtcagagggagagtggctgcggaggaagaaga
ctttag
SEQ ID NO: 2
>CaDREB-LP1 (NM_001324857.1)
ATGAACATCTTTAGAAGCTATTATTCGGACCCACTTACTGAATCTTCATCATCTTTTTCTGATAGTAGCATTTACTCCCCTAATAGA
GCTATTTTTTCTGATGAGGAAGTTATATTAGCATCAAATAACCCGAAAAAGCCAGCTGGGAGGAAGAAGTTTCGAGAAACTCGACAT
CCAGTATACAGGGGAGTTAGGAAGAGGAATTCAGGCAAATGGGTTTGTGAAGTCAGAGAACCCAATAAGAAATCAAGAATTTGGCTT
GGTACTTTTCCTACAGCTGAAATGGCTGCTAGAGCTCATGACGTGGCGGCTATAGCATTAAGAGGTCGTTCTGCTTGTTTGAACTTT
GCTGATTCTGCTTGGAGGTTGCCTGTTCCGGCTTCCTCTGACACTAAAGATATTCAAAAGGCGGCCGCTGAGGCCGCGGAAGCCCTC
CGACCATTGAAGTTGGAAGGAATTTCAAAAGAATCATCTAGCAGTACTCCAGAGAGTATGTTCTTTATGGATGAGGAAGCGCTCTTC
TGCATGCCGGGATTACTTACGAATATGGCTGAAGGGCTAATGTTACCACCACCTCAATGTGCAGAAATTGGAGATCATGTGGAAACT
GCTGATGCGGATACCCCTTTATGGAGCTATTCCATTTAA
SEQ ID NO: 3
>AmROS1(DQ275529.1)
atggaaaagaattgtcgtggagtgagaaaaggtacttggaccaaagaagaagacactctcttgaggcaatgtatagaagagtatggt
gaagggaaatggcatcaagttccacacagagcagggttgaaccggtgtaggaagagttgcaggctgaggtggttgaattatctgagg
ccaaatatcaaaagaggtcggttttcgagagatgaagtggacctaattgtgaggcttcataagctgttgggtaacaaatggtcgctg
attgctggtagaattcctggaaggacagctaatgacgtgaagaacttttggaatactcatgtggggaagaatttaggcgaggatgga
gaacgatgccggaaaaatgttatgaacacaaaaaccattaagctgactaatatcgtaagaccccgagctcggaccttcaccggattg
cacgttacttggccgagagaagtcggaaaaaccgatgaattttcaaatgtccggttaacaactgatgagattccagattgtgagaag
caaacgcaattttacaatgatgttgcgtcgccacaagatgaagttgaagactgcattcagtggtggagtaagttgctagaaacaacg
gaggatggggaattaggaaacctattcgaggaggcccaacaaattggaaattaa
SEQ ID NO: 4
>GmLOG1(XM_003527643.3)
ATGGAAACTCAACACCAACAACCCACCATCAAGTCTAGGTTCAGACGCATCTGTGTCTACTGTGGTAGCAGCCCTGGCAAAAACCCC
AGCTACCAGCTCGCTGCTATTCAACTCGGAAAACAACTGGTGGAGAGGAACATTGACTTGGTTTATGGAGGAGGAAGCATAGGGTTG
ATGGGTCTAATCTCACAAGTTGTGTATGATGGTGGACGCCACGTGTTAGGGGTGATTCCAGAGACACTTAATGCAAGAGAGATAACT
GGAGAGAGTGTTGGAGAAGTGAGAGCTGTATCGGGCATGCACCAACGCAAAGCCGAAATGGCCCGACAAGCCGATGCATTTATTGCA
CTGCCAGGTGGATATGGCACCCTTGAAGAACTACTGGAAATTATCACCTGGGCTCAACTAGGCATCCATGATAAACCGGTGGGGTTG
TTGAACGTGGATGGGTACTACAACTCGCTGCTGGCATTCATGGACAAAGCTGTGGACGAAGGTTTCGTAACACCAGCTGCCCGTCAC
ATTATTGTTTCTGCCCACACTGCCCAAGAACTCATGTGCAAACTTGAGGAATATGTCCCCGAGCACTGTGGCGTGGCCCCCAAGCTA
AGTTGGGAGATGGAGCAACAGTTAGTTAACACTGCAAAGTCAGATATTTCCCGTTGA
SEQ ID NO: 5
>SILOG1 (NM_001324502.1)
ATGGAAAACAATCACCAGACACAAATTCAGACCACTAAAACATCAAGATTCAAACGCATATGTGTTTTTTGTGGAAGCAGTCCAGGC
AAAAAGCCAAGTTATCAACTTGCTGCTATTCAACTTGGCAATCAACTGGTTGAAAGGAACATCGACTTGGTTTATGGAGGTGGCAGT
GTGGGCTTGATGGGCCTAGTTTCTCAATCAGTTTTTAATGGTGGCCGCCACGTGTTAGGGGTGATTCCTAAAACTCTTATGCCAAGA
GAGATTACTGGAGAAAGTGTTGGAGAAGTAAGAGCAGTGTCTGGGATGCATCAAAGAAAAGCAGAAATGGCAAGACAAGCTGATGCA
TTCATAGCCTTACCAGGTGGCTATGGGACATTGGAAGAGCTCCTAGAAGTCATCACTTGGGCTCAACTAGGCATTCATGATAAACCA
GTAGGTTTACTTAATGTAGATGGCTACTATAATTCATTATTATCATTTATAGACAAAGCTGTTGATGAAGGCTTTGTCACACCCTCT
GCCCGTCACATCATTATTTCTGCCCCAACTGCCCAAGAACTCATGTCTAAGCTTGAGGATTATGTACCAAAGCATAATGGGGTGGCA
CCAAAATTGAGTTGGGAAATGGAACAACAACTTGGCTACACAACAACAAAATTGGAAATTGCTCGTTAA
SEQ ID NO: 6
>sGFP (U43284.1), nucleotide positions 826-1545/nucleotide exchanges C96T and T695A
atggtgagcaagggcgaggagctgttcaccggggtggtgcccatcctggtcgagctggacggcgacgtaaacggccacaagttcagc
gtgtccggcgagggcgagggcgatgccacctacggcaagctgaccctgaagttcatctgcaccaccggcaagctgcccgtgccctgg
cccaccctcgtgaccaccttcagctacggcgtgcagtgcttcagccgctaccccgaccacatgaagcagcacgacttcttcaagtcc
gccatgcccgaaggctacgtccaggagcgcaccatcttcttcaaggacgacggcaactacaagacccgcgccgaggtgaagttcgag
ggcgacaccctggtgaaccgcatcgagctgaagggcatcgacttcaaggaggacggcaacatcctggggcacaagctggagtacaac
tacaacagccacaacgtctatatcatggccgacaagcagaagaacggcatcaaggtgaacttcaagatccgccacaacatcgaggac
ggcagcgtgcagctcgccgaccactaccagcagaacacccccatcggcgacggccccgtgctgctgcccgacaaccactacctgagc
acccagtccgccctgagcaaagaccccaacgagaagcgcgatcacatggtcctgctggagttcgtgaccgccgccgggatcactcac
ggcatggacgagctgtacaagtaa
SEQ ID NO: 7
>SIGR (XM_010325884.2)
atgctgccaagaagatatcctcagatggatgctaatcctagtaatgggggtgaaagggataatgctttgcgaggaattctgcaggac
ttatggccactggatgaaattgatccaagcactcaaaagttcccttgttgccttgtttggactcctctccctgtgatttcttggctt
gcaccttttgttggacatgttggcatatgcagggaggatggtaccattgtggatttttctggagatagcatgattcattttggtcag
ctcttctatggaactgtagccaaatactatcaggtagacagacagcagtgctgttttgctcgcaactttggtggacacacatgccgt
aagggttatgaacatgttgtatttgggacagcagtaagttgggatgatgctgttcagttgtttaggcgcacctttgagaacagaaac
ttcaaagttttcagttgcaacggccactcattcgctgctgattgcctgaacctgctatcatttagaggatcaatgcgctggaacatg
attaatgttggagctcttataatgtttgagggaaagtgggtcagtcgctggtcaatgttacgatcatttctgcctttcattgggata
ctttgcttcggctatttaatgattggatggatgtttccaattggtctgctctccatgttattgggacttttggatggtatgtcatga
tctgttactgttgcaagattgaggatgacaattag
SEQ ID NO: 8
>SIDREB1 (NM_001247760.1)
ATGGCTATTATGGATGAAGCTGCTAATATGGTTTGTGTGCCGTTGGATTATAGTAGAAAGAGGAAATCAAGGAGTAGAAGGGACAGA
ACAAAAAATGTGGAAGAGACACTAGCTAAATGGAAGGAGTATAATGAGAAACTAGACAATGAAGGGAAAGGGAAGCCAGTGCGTAAA
GTTCCTGCTAAAGGTTCAAAGAAGGGGTGTATGAGAGGTAAAGGGGGACCAGAAAATTGGCGGTGTAAATACAGAGGTGTTAGACAG
AGGATATGGGGTAAATGGGTTGCTGAGATTAGGGAACCTAAAAGAGGTAGTAGGTTATGGTTGGGTACATTTGGTACAGCAATTGAA
GCTGCTTTAGCATATGATGATGCTGCAAGAGCTATGTATGGTCCTTGTGCAAGGCTTAATTTGCCAAATTACGCGTGTGATTCTGTT
TCCTGGGCAACTACATCTGCATCTGCATCTGCATCTGATTGCACCGTTGCTTCTGGTTTCGGCGAGGTATGTCCGGTTGATGGTGCT
CTTCATGAAGCTGACACACCATTGAGCTCAGTGAAAGACGAAGGGACCGCGATGGATATTGTTGAACCTACGAGTATTGATGAAGAT
ACGCTTAAGTCTGGATGGGATTGTCTAGATAAATTAAATATGGATGAGATGTTTGATGTAGATGAGCTATTGGCTATGTTAGATTCT
ACTCCAGTTTTCACCAAGGACTACAATTCAGATGGAAAGCACAACAATATGGTATCAGATTCGCAATGTCAGGAGCCGAATGCAGTG
GTAGATCCTATGACTGTTGACTATGGCTTTGATTTTCTGAAACCAGGCAGGCAAGAAGATCTTAATTTCAGTTCGGATGACCTTGCA
TTCATAGACTTGGATTCTGAACTTGTCGTTTGA
SEQ ID NO: 9
>SIOVATE(NM_001247292.2)
ATGGGAAAAAGTTTGAAGCTTCGGTTCTCCAGAGTTATTGCTTCTTTCAATTCGTGCCGTTCGAAAAACCCTTCTTCTCTTCCCCAA
AATCCTAATTTCTTCCCACATAAGCTCACTAGTACAAAACACATTTCCCCCGATTTCCCTCTTATTGATCAAAATCAAAATCAAAAT
CACCGTAATTACGTGCCAGAATCCACGATGATCTCCGTTGGGTGTTGTAGATCAGAATTCAAGTGGGAGAAAGAAGAGAAGTTTCAC
GTGGTTTCTAGTTCCTTCGTGTCTGAAGAAGAAGAATGTGAAGAGGAGATCAATTTGGCCTTACGACCTCCTCTTACACCTCCGCGA
TTCAGTAGAATTGTTGTTGAGAAGAAGAAGAAGAAACAACAGCGAGTTAAAAAAACGAAAACAAAAAGTAGAATCATCCGAATGAGT
ACTTCCTCAGCTGATGAGTACAGCGGGATATTAAGCGGTACTAATACTGATTGGGATAATAATGAAGAGGAAACTGAATCTTTAGTT
TCATCTTCCAGAAGCTGTTACGATTTCTCAAGCGATGACTCATCTACTGATTTCAACCCTCACTTAGAAACCATATGTGAGACCACT
ACAATGAGGCGTCGTCACAAGAGAAATGCCAACACCAAGAGGAGATCAATCAAGCAATCCAGACCAAGTTTTTCCTCTTCAAAAGGT
AGAAGATCGTCGGTTTCTACGTCATCAGATAGCGAGCTACCGGCAAGGTTATCGGTGTTTAAGAAGCTGATACCGTGTAGTGTGGAT
GGGAAAGTGAAGGAGAGTTTCGCGATAGTGAAGAAATCTCAGGACCCGTACGAAGATTTCAAGAGATCGATGATGGAAATGATTTTA
GAGAAGGAAATGTTTGAGAAGAATGAGCTGGAACAGCTTTTACAATGTTTTCTGTCGTTGAACGGAAAGCATTATCATGGAGTGATA
GTTGAGGCGTTCTCAGACATTTGGGAGACTTTGTTTTTAGGTAATAATGATAGAGTAAGGAGGATGTCAATTCATGATCCCACACCC
ACCTACTGTAGGTAG
SEQ ID NO: 10
>SISUN (NM_001246864.2)
ATGGGAAAGCGAAGAAACTGGTTTACCTTTGTCAAGAGACTTTTCATTCCTGAAACAGAATCAACAGCAGATCAAAAGAAACCAAAG
AGATGGAGATGTTGTTTTCTGAGAAAGTTCAAGTTGAGGAAATGTCCTGCTATAACATCAGCACCTCAGCAAACGTTACCTGAGGCG
AAAGGAACACCTCAGCAAACGTTAACTGAGGCGAAAGAACAGCAAAGAAAACATGCTTTTGCAGTTGCTATAGCAACGGCAGCAGCT
GCTGAGGCTGCTGTAGCTGCTGCTAATGCTGCTGCTGATGTTATTCGTCTAACAGATGCTCCAAGTGAATTCAAAAGGAAACGCAAA
CAAGCTGCTATTAGAATCCAAAGTGCTTATCGCGCTCACCTGGCCCAGAAAGCATTAAGGGCTCTAAAGGGTGTTGTGAAGCTTCAA
GCAGTGATTAGAGGTGAAATTGTGAGAGGAAGACTCATTGCCAAACTGAAGTTCATGTTGCCACTTCATCAAAAGTCAAAAACAAGA
GTTAATCAAATTAGAGTCCCTACTTTTGAAGATCATCATGACAAGAAACTCATCAATAGTCCAAGGGAAATTATGAAAGCTAAAGAA
CTAAAGCTTAAATGCAAGAGCCTTAGCACTTGGAATTTCAACTTAGCTTCAGAACAAGACAGTGAAGCCTTGTGGTCAAGAAGAGAA
GAAGCCATTGACAAAAGAGAGCATTTGATGAAATACTCGTTTTCACATCGGGAGAGAAGAAACGATCAAACTCTACAAGACTTACTA
AACAGAAAGCAAAACAGAAGAAGCTACAGGATTGACCAGTTAGTAGAACTTGACGCACCAAGAAAAGCAGGGTTGTTAGAGAAATTG
AGATCATTTACAGACTCAAATGTTCCTCTAACTGATATGGATGGAATGACACAGCTTCAAGTGAGAAAAATGCATAGATCAGATTGT
ATAGAGGACCTACATTCTCCTTCTTCACTTCCAAGAAGATCATTTTCTAATGCAAAACGAAAATCAAACGTTGATGATAACTCATTA
CCAAGTTCTCCTATATTTCCTACTTACATGGCAGCCACAGAATCTGCAAAGGCAAAAACAAGGTCAAACAGCACAGCGAAGCAACAC
CTAAGGTTACACGAGACATTGTCAGGTCAACATTCTCCTTATAACCTCAAGATTTCTTCTTGGAGATTGTCTAATGGTGAAATGTAT
GACAGCGCCAGAACAAGCAGAACTTCTAGCAGTTATATGTTAATATAG
SEQ ID NO: 11
>GUS (S69414.1)/nucleotide exchanges G835C and G903A
atgttacgtcctgtagaaaccccaacccgtgaaatcaaaaaactcgacggcctgtgggcattcagtctggatcgcgaaaactgtgga
attgatcagcgttggtgggaaagcgcgttacaagaaagccgggcaattgctgtgccaggcagttttaacgatcagttcgccgatgca
gatattcgtaattatgcgggcaacgtctggtatcagcgcgaagtctttataccgaaaggttgggcaggccagcgtatcgtgctgcgt
ttcgatgcggtcactcattacggcaaagtgtgggtcaataatcaggaagtgatggagcatcagggcggctatacgccatttgaagcc
gatgtcacgccgtatgttattgccgggaaaagtgtacgtatcaccgtttgtgtgaacaacgaactgaactggcagactatcccgccg
ggaatggtgattaccgacgaaaacggcaagaaaaagcagtcttacttccatgatttctttaactatgccggaatccatcgcagcgta
atgctctacaccacgccgaacacctgggtggacgatatcaccgtggtgacgcatgtcgcgcaagactgtaaccacgcgtctgttgac
tggcaggtggtggccaatggtgatgtcagcgttgaactgcgtgatgcggatcaacaggtggttgcaactggacaaggcactagcggg
actttgcaagtggtgaatccgcacctctggcaaccgggtgaaggttatctctatgaactgtgcgtcacagccaaaagccagacagag
tgtgatatctacccgcttcgcgtcggcatccggtcagtggcagtgaagggccaacagttcctgattaaccacaaaccgttctacttt
actggctttggtcgtcatgaagatgcggacttacgtggcaaaggattcgataacgtgctgatggtgcacgaccacgcattaatggac
tggattggggccaactcctaccgtacctcgcattacccttacgctgaagagatgctcgactgggcagatgaacatggcatcgtggtg
attgatgaaactgctgctgtcggctttaacctctctttaggcattggtttcgaagcgggcaacaagccgaaagaactgtacagcgaa
gaggcagtcaacggggaaactcagcaagcgcacttacaggcgattaaagagctgatagcgcgtgacaaaaaccacccaagcgtggtg
atgtggagtattgccaacgaaccggatacccgtccgcaaggtgcacgggaatatttcgcgccactggcggaagcaacgcgtaaactc
gacccgacgcgtccgatcacctgcgtcaatgtaatgttctgcgacgctcacaccgataccatcagcgatctctttgatgtgctgtgc
ctgaaccgttattacggatggtatgtccaaagcggcgatttggaaacggcagagaaggtactggaaaaagaacttctggcctggcag
gagaaactgcatcagccgattatcatcaccgaatacggcgtggatacgttagccgggctgcactcaatgtacaccgacatgtggagt
gaagagtatcagtgtgcatggctggatatgtatcaccgcgtctttgatcgcgtcagcgccgtcgtcggtgaacaggtatggaatttc
gccgattttgcgacctcgcaaggcatattgcgcgttggcggtaacaagaaagggatcttcactcgcgaccgcaaaccgaagtcggcg
gcttttctgctgcaaaaacgctggactggcatgaacttcggtgaaaaaccgcagcagggaggcaaacaatga
SEQ ID NO: 12
>SIWoolly (XM_004232686.3)
atgtttaataaccaccagcacttgctcgatatatcgtcctcagctcaacgaacacctgataacgagttggatttcattcgtgatgaa
gagtttgatagcaactctggtgctgataacatggaagctcccaattcaggtgatgacgatcaagctgatccaaaccaacctccaaac
aagaagaagcgttatcatcgccacactcagaatcagattcaggaaatggagtccttttacaaggaatgcaatcatccagatgacaag
caaaggaaggaattgggaagaagacttggtttggagccattacaagtgaaattttggttccagaacaagcgtactcagatgaaggct
caacatgagcgatgtgagaacacacagttgaggaatgaaaatgagaagcttcgcgctgagaacataaggtacaaagaagctttgagt
aatgcagcatgcccaaattgtggagggccagcagctataggagagatgtcatttgatgagcatcagttgaggattgaaaatgctcgt
cttagagatgagattgacaggataactggaatagctggaaagtatgttggtaaatcagcccttggatattctcatcaacttcctctt
cctcagcccgaagctcctcgggttctggatcttgcttttgggcctcaatcgggcctgcttggagaaatgtacgctgctggtgacctt
ctaagaactgctgttacgggccttacagatgctgagaagcccgtggtcattgagcttgctgttactgcaatggaggaacttataagg
atggctcaaactgaagagccattatggttgccaagctcaggctctgagactttatgtgagcaagaatatgctcgtattttccctcga
ggccttggacctaagccagctacactcaattctgaagcctcacgagaatctgctgttgtgattatgaatcatatcaatttagttgag
attttgatggatgtgaaccaatggactactgtttttgctggtctggtgtcaaaagcaatgactcttgaagtcttatcaactggtgtc
gcaggaaatcacaatggagcattgcaagtgatgacagcagaatttcaagttccatctccacttgttccaactcgggagaactatttc
ttaagatactgtaaacaacatggtgaagggacttgggtagtggttgatgtttccctggacaacttgcgcactgtttcagttccgcgt
tgcagaagaaggccatctggttgtttaatccaagaaatgccaaatggttactcaagggttatatgggttgaacacgttgaggtggat
gaaaatgctgtccatgacatctacaaacctcttgtcaattctgggattgcatttggagcaaaacgctgggtagcaactttagataga
caatgtgaacgccttgcaagtgtgttggcgcttaacatcccaacaggagatgttggaatcattactagtccagctggtcgaaagagt
atgctaaaacttgctgagagaatggtgatgagcttttgtgctggagttggtgcatcgacaactcacatatggacaactttgtctgga
agtggtgcggatgatgttagagtcatgactaggaagagtatcgatgatccagggagacctcctggtattgtgctgagtgctgcaaca
tctttttggcttccagtttctcctaagagagtgtttgattttctccgcgatgagaactctagaaatgagtgggatattctttcaaat
ggtgggattgttcaggaaatggcacacattgcaaatggtcgtgatccaggaaactgtgtttctctactccgtgtcaatactggaaca
aactctaaccagagtaacatgctgatactccaagagagcacaactgatgtaacaggatcttacgtcatttacgctccagttgatatt
gctgcaatgaacgtggtgttaggtgggggtgaccctgactatgttgctctgttgccatctggttttgctattcttccagacggaccg
atgaattatcatggtggaggtaattcagaaattgattctcctggtggatcgctactaactgtagcatttcagatattggttgattca
gtcccaactgcaaagctttcccttggctctgttgcgactgttaatagtctcatcaaatgcaccgttgaaaagatcaaaggtgctgta
acttccgcaaatgcatga
SEQ ID NO: 13
>SIANT1(NM_001247488.1) native sequence from Solanum lycopersicum
atgaacagtacatctatgtcttcattgggagtgagaaaaggttcatggactgatgaagaagattttcttctaagaaaatgtattgat
aagtatggtgaaggaaaatggcatcttgttcccataagagctggtctgaatagatgtcggaaaagttgtagattgaggtggctgaat
tatctaaggccacatatcaagagaggtgactttgaacaagatgaagtggatctcattttgaggcttcataagctcttaggcaacaga
tggtcacttattgctggtagacttcccggaaggacagctaacgatgtgaaaaactattggaacactaatcttctaaggaagttaaat
actactaaaattgttcctcgcgaaaagattaacaataagtgtggagaaattagtactaagattgaaattataaaacctcaacgacgc
aagtatttctcaagcacaatgaagaatgttacaaacaataatgtaattttggacgaggaggaacattgcaaggaaataataagtgag
aaacaaactccagatgcatcgatggacaacgtagatccatggtggataaatttactggaaaattgcaatgacgatattgaagaagat
gaagaggttgtaattaattatgaaaaaacactaacaagtttgttacatgaagaaatatcaccaccattaaatattggtgaaggtaac
tccatgcaacaaggacaaataagtcatgaaaattggggtgaattttctcttaatttaccacccatgcaacaaggagtacaaaatgat
gatttttctgctgaaattgacttatggaatctacttgattaa
SEQ ID NO: 14
>SIANT1 with Nicotiana tabacum codon usage
atgaattctacaagtatgtcaagcttaggcgttcgtaagggatcttggacagatgaagaagatttccttctacgaaagtgtattgac
aaatatggtgagggaaaatggcatttggttccgattagagctggtttgaatcgatgcaggaaatcctgtagacttaggtggttgaac
tatcttagacctcacataaagagaggtgatttcgagcaagatgaagtggatctcatactcagactacacaaacttttagggaatcgt
tggagtcttattgcaggcagattaccaggtagaacagccaatgatgtcaagaactattggaatactaatcttttaaggaagttgaac
actacaaagatagtaccaagggagaaaatcaacaacaaatgtggggaaatttctacgaaaattgagattatcaagccccaaagacgt
aagtacttttcatccactatgaagaatgtcaccaacaacaatgttatcctcgacgaagaagaacattgcaaagagatcatttctgag
aagcagactcctgatgcttcaatggacaacgttgatccttggtggataaatcttctagagaattgcaacgatgatatagaagaggat
gaagaagtggtgattaactacgagaaaaccttaactagcctgttgcatgaagaaatctctccaccccttaatattggagaaggaaat
tcaatgcaacaaggccagatttctcatgagaattggggtgaattttccttgaatctgccacctatgcagcaaggagtacagaatgac
gactttagtgcagagattgatctctggaatctgttggactaa
SEQ ID NO: 15
>SIANT1 with Arabidopsis thaliana codon usage
atgaattcaacatcaatgtctagtctaggagtaaggaaaggttcatggacagatgaagaggactttcttctccggaaatgcattgat
aagtatggggaaggaaaatggcatttagtccccattagagctggcttgaatcgttgtaggaaatcgtgtcgactcagatggctaaac
tatcttagaccgcatatcaagcggggtgatttcgaacaggacgaagtggacttgattttgaggcttcacaagttattgggtaatcgt
tggtcccttatagctgggagattaccaggtagaacagccaatgatgtgaagaattactggaatacgaacttgctgagaaaactcaac
actaccaagatcgttccgagagaaaagatcaacaacaaatgtggcgagattagcacgaagatagagatcataaagcctcaacgtcga
aaatacttctctagcactatgaagaatgtcaccaataacaacgtgatactagatgaagaagaacactgtaaggagattatcagtgag
aaacagactcctgatgcatctatggacaatgttgatccttggtggattaaccttctggagaattgcaatgacgatattgaggaggat
gaagaggttgtaatcaactatgagaaaacacttacttcactccttcatgaagagatatctccaccacttaacattggagagggtaac
tccatgcaacaaggacagatctctcatgaaaattggggagaattttcgctgaatttgcctccaatgcaacaaggagttcagaacgac
gattttagtgcggaaattgatctctggaacttattggattaa
SEQ ID NO: 16
>SIANT1 with Potato Virus X codon usage
atgaatagcactagcatgtcaagcttaggtgtgagaaagggctcatggactgacgaagaggatttcctgttgaggaagtgcatcgac
aagtatggagaaggcaaatggcaccttgtaccgattagggcagggcttaacaggtgcaggaaaagctgtaggttgaggtggttgaac
tatctcagaccccatataaagagaggcgactttgagcaagatgaagtggacctaattcttcgcttacacaaactccttgggaatagg
tggagtctgatagctggaaggctacctggtagaacagctaacgacgtgaagaactactggaataccaacctattacgcaaactgaac
actaccaaaatcgttcccagagagaagatcaacaacaagtgtggcgagataagcacgaagatcgaaatcatcaaaccgcaaagaagg
aagtacttcagttcaaccatgaagaatgtcacaaacaacaatgtcatactggatgaagaagagcactgcaaggagattatttccgag
aaacagacaccagacgcatccatggacaatgtcgatccatggtggattaacctactcgaaaattgcaacgatgacattgaagaggat
gaggaagtagtgatcaactacgagaaaacactgacttctctcttgcatgaggagatcagtccacctttgaacattggagaagggaat
tctatgcaacaaggacagataagccacgaaaattggggagagttttccctcaatctcccacctatgcaacagggtgttcagaacgat
gacttctcagccgaaatcgacttatggaacctactcgactaa
SEQ ID NO: 17
>SIANT1 with Homo sapiens codon usage
atgaattctacgtccatgtctagcctcggggttaggaaaggctcatggacagacgaagaggactttctgctgcgcaaatgcatagac
aagtatggcgaaggaaagtggcatctggtgcccattagggctggtctgaaccggtgtcgcaagtcctgtaggttgcggtggcttaac
tacctcagaccccacatcaaacgaggcgatttcgaacaggatgaggtcgacctgattctccgtctgcacaagctgttgggtaacaga
tggagcctcattgcagggagactccctggaagaactgccaatgacgtcaagaactactggaacaccaaccttcttcgcaagctgaat
accactaagatcgttcctcgagagaagatcaacaacaaatgtggagaaatatccaccaaaatcgagatcatcaagccacaacggagg
aaatacttctccagcacaatgaagaatgtgaccaacaacaacgtgattttggacgaagaggagcattgcaaagagatcatcagtgag
aagcagacacctgatgcctctatggataatgtggacccctggtggataaatctgctggagaattgcaatgatgacattgaagaagat
gaggaagtggtcatcaactatgagaaaacactgacttcactgctgcatgaagagattagtccaccgctgaacattggggaggggaat
agcatgcagcagggacagatcagtcacgaaaattggggcgaattcagccttaatctcccacccatgcaacagggcgtacagaacgac
gacttttcagcggagattgatctgtggaatttgctggattaa
SEQ ID NO: 18
>SIANT1 with Oryza sativa codon usage
atgaattcaacgagcatgagctcgttgggtgttcgcaaaggctcttggaccgatgaagaggacttcctcttgcgaaagtgcatcgat
aagtatggggaaggaaagtggcatcttgtacccatacgtgcgggacttaaccggtgtcgcaagtcgtgcagactcaggtggctcaac
tatctacggcctcacatcaaacgtggcgatttcgaacaagacgaggttgaccttatcctgagactgcacaaactgctcggcaatcgc
tggagtctcatagctggtcgattgcctgggaggactgccaatgacgtcaagaattactggaatacaaaccttctgaggaagctgaat
accacgaagatagttcctcgggagaagatcaacaacaagtgtggggagatttccacgaaaatcgagatcatcaagccgcaaaggcgc
aaatacttctcaagcacaatgaagaacgtcaccaacaacaacgtgattctcgatgaggaggaacactgcaaggagatcatctctgag
aaacagactccagatgcctcaatggacaatgtggatccgtggtggattaacctcctggagaactgcaatgatgacattgaagaggac
gaagaggtcgtgatcaactacgaaaagaccctcacatctctcctccatgaggaaataagtccaccgctcaatattggcgaaggcaat
tccatgcagcaaggccagatttcgcatgagaactggggtgagttttccctgaatctaccacccatgcagcaaggagtgcagaatgat
gacttttccgcagagattgacttgtggaacttgcttgattaa
SEQ ID NO: 19
>SIANT1 with Hordeum vulgare codon usage
atgaatagcacctccatgtcctctctgggcgttcgtaaggggtcatggacagatgaggaggacttcttgctccgcaaatgcatcgac
aagtatggcgaaggcaaatggcatcttgtcccgataagggccggactcaaccgctgcagaaagtcttgccgccttaggtggctaaac
tacctacggccccacattaagcggggtgactttgagcaggatgaggtagacttgatcttgcggctacacaagcttctgggcaatagg
tggtcactgattgccggtagactccctggtcgcactgcgaatgacgtgaagaactactggaacaccaatctgctccgcaaactcaac
accaccaagatcgtcccacgtgagaagatcaacaacaagtgtggcgagatcagcaccaagatcgagatcatcaagccacaacggagg
aagtacttctcctctacgatgaagaatgtgacgaacaacaacgtgattctcgacgaagaggagcactgtaaggagatcatctccgag
aaacagactcccgatgcttcgatggacaatgtcgatccgtggtggattaacctcctggagaattgcaacgatgacatagaagaggac
gaagaagtcgtgatcaactacgaaaagacgctgacaagcctcttgcacgaggagatatcgccacccctcaacattggagaggggaac
agcatgcagcaagggcagatcagtcatgaaaactggggagagttcagcctcaatcttcctccgatgcagcaaggcgttcagaacgat
gacttcagtgcagagattgacctgtggaaccttctcgattaa
SEQ ID NO: 20
>SIANT1 with Bifidobacterium codon usage
atgaactccacctccatgtcctcgctcggcgttcgcaaaggcagctggaccgatgaggaggacttcctcctgcgcaagtgcatcgac
aagtacggagaaggcaaatggcaccttgtccccattcgcgctggtctgaaccgctgtcgcaagagctgccgtttgcggtggctgaac
tatctgcgtccgcacatcaagcgcggcgacttcgagcaggacgaagtcgacctgattctgcgcctgcataagctgctggggaaccgc
tggtccctgattgccggccggttgcccggtaggaccgcgaacgacgtgaagaactactggaacaccaacctccttcgcaagctgaat
accacgaagatcgtgccgagggagaagatcaacaacaaatgcggggaaatctcgacgaagatcgagatcatcaagccccaacgtcgg
aagtacttcagcagcaccatgaagaacgtgacgaacaacaacgtgatcctggacgaagaggaacactgcaaggagatcatctcggag
aagcagactccggatgcctccatggacaacgtggatccgtggtggatcaatctgctggagaactgcaacgacgacatcgaggaggat
gaggaagtcgtgatcaactacgaaaagaccttgacgtccctcctccatgaggagatttcccctccgctgaacatcggcgagggcaac
tccatgcaacagggccagatctcccacgagaattggggcgaattctcgctgaatctcccgccgatgcagcagggagtccagaacgac
gactttagcgccgaaatcgacctctggaaccttctcgattaa
SEQ ID NO: 21
>SILOG1 with Oryza sativa codon usage
atggagaacaaccatcaaacgcagattcagactaccaagacttctcgcttcaagcgcatttgcgtgttctgtgggtcaagtccaggc
aagaagccctcctatcagcttgctgccatccagctggggaatcagctggttgaacggaatatcgatctcgtctatggtggaggctct
gttggcctaatgggactcgtgagccaatccgtgttcaatggtggtcgacatgtcctcggcgtgataccgaaaaccctgatgcccaga
gagatcacgggagagtcagtcggagaagtccgggctgtttctggcatgcatcagaggaaagccgagatggcacgtcaagccgatgcg
tttatagcgcttcctggcggttacggaaccctcgaagagctactggaggtgattacatgggctcagttgggcatacacgacaaacca
gttggcctcttgaacgtggatgggtactacaactcgttgctttcgttcatcgacaaggcagtagacgaggggtttgtgacaccatcc
gcaagacacatcatcattagtgcgcctacagcccaagaactcatgagcaagcttgaggactatgtcccgaagcacaatggggtagcc
ccgaaactgagctgggagatggaacaacagctcggctacacgactaccaagctcgagattgcgaggtga
SEQ ID NO: 22
>SIOVATE with Oryza sativa codon usage
atgggcaaaagtctcaagctgcgcttttctcgtgtgattgccagcttcaattcgtgcagatctaagaatcccagctcacttccgcaa
aatccgaacttctttccccacaagcttacatcgacaaaacacatctctccagactttccgctgattgaccagaaccagaaccagaat
cacaggaactacgttcctgagtcgaccatgatcagtgtgggctgttgcagatccgaattcaagtgggagaaagaggagaagtttcac
gtggtatcaagctcgttcgtttccgaggaagaggagtgtgaagaagagatcaaccttgctctacgtccaccgctaacaccaccgcgc
ttctcaaggatagttgtcgagaagaagaagaagaaacagcaacgggtgaagaaaacgaaaaccaaatcccgcatcattcgcatgtcc
acttcatctgcggatgagtacagtgggatcttgagcggtaccaacacagattgggacaacaatgaggaggaaaccgaaagtctggtg
tccagctcaaggagctgttacgacttctcgagtgatgactcgtccacggatttcaatccgcatttggagactatttgcgaaactacg
acaatgagaaggcggcataaaaggaatgccaacacgaagcgacgctctatcaaacaaagccgaccttcattctcctcaagcaaggga
cgcagaagctccgtgtcgacctcctcagactctgagctcccagctaggctcagtgtctttaagaagctcattccttgctctgtggat
ggaaaggtcaaggagtccttcgcaatcgtcaagaaatcgcaagatccctatgaggacttcaagcggtctatgatggagatgatcctg
gagaaggaaatgtttgagaagaatgagctcgaacagcttctccagtgcttcctctccctcaacggcaagcattaccatggtgtcata
gttgaagcgtttagcgacatatgggaaacgctgttcttggggaataacgatcgggtacgtcgaatgagcattcacgatcctactccc
acctattgccggtga
SEQ ID NO: 23
>pNMD674
cttctgtcagcgggcccactgcatccaccccagtacattaaaaacgtccgcaatgtgttattaagttgtctaagcgtcaatttgttt
acaccacaatatatcctgccaccagccagccaacagctccccgaccggcagctcggcacaaaatcaccactcgatacaggcagccca
tcagtcagatcaggatctcctttgcgacgctcaccgggctggttgccctcgccgctgggctggcggccgtctatggccctgcaaacg
cgccagaaacgccgtcgaagccgtgtgcgagacaccgcggccgccggcgttgtggatacctcgcggaaaacttggccctcactgaca
gatgaggggcggacgttgacacttgaggggccgactcacccggcgcggcgttgacagatgaggggcaggctcgatttcggccggcga
cgtggagctggccagcctcgcaaatcggcgaaaacgcctgattttacgcgagtttcccacagatgatgtggacaagcctggggataa
gtgccctgcggtattgacacttgaggggcgcgactactgacagatgaggggcgcgatccttgacacttgaggggcagagtgctgaca
gatgaggggcgcacctattgacatttgaggggctgtccacaggcagaaaatccagcatttgcaagggtttccgcccgtttttcggcc
accgctaacctgtcttttaacctgcttttaaaccaatatttataaaccttgtttttaaccagggctgcgccctgtgcgcgtgaccgc
gcacgccgaaggggggtgcccccccttctcgaaccctcccggcccgctaacgcgggcctcccatccccccaggggctgcgcccctcg
gccgcgaacggcctcaccccaaaaatggcagcgctggccaattcgtgcgcggaacccctatttgtttatttttctaaatacattcaa
atatgtatccgctcatgagacaataaccctgataaatgcttcaataatattgaaaaaggaagagtatggctaaaatgagaatatcac
cggaattgaaaaaactgatcgaaaaataccgctgcgtaaaagatacggaaggaatgtctcctgctaaggtatataagctggtgggag
aaaatgaaaacctatatttaaaaatgacggacagccggtataaagggaccacctatgatgtggaacgggaaaaggacatgatgctat
ggctggaaggaaagctgcctgttccaaaggtcctgcactttgaacggcatgatggctggagcaatctgctcatgagtgaggccgatg
gcgtcctttgctcggaagagtatgaagatgaacaaagccctgaaaagattatcgagctgtatgcggagtgcatcaggctctttcact
ccatcgacatatcggattgtccctatacgaatagcttagacagccgcttagccgaattggattacttactgaataacgatctggccg
atgtggattgcgaaaactgggaagaagacactccatttaaagatccgcgcgagctgtatgattttttaaagacggaaaagcccgaag
aggaacttgtcttttcccacggcgacctgggagacagcaacatctttgtgaaagatggcaaagtaagtggctttattgatcttggga
gaagcggcagggcggacaagtggtatgacattgccttctgcgtccggtcgatcagggaggatatcggggaagaacagtatgtcgagc
tattttttgacttactggggatcaagcctgattgggagaaaataaaatattatattttactggatgaattgttttagctgtcagacc
aagtttactcatatatactttagattgatttaaaacttcatttttaatttaaaaggatctaggtgaagatcctttttgataatctca
tgaccaaaatcccttaacgtgagttttcgttccactgagcgtcagaccccgtagaaaagatcaaaggatcttcttgagatccttttt
ttctgcgcgtaatctgctgcttgcaaacaaaaaaaccaccgctaccagcggtggtttgtttgccggatcaagagctaccaactcttt
ttccgaaggtaactggcttcagcagagcgcagataccaaatactgtccttctagtgtagccgtagttaggccaccacttcaagaact
ctgtagcaccgcctacatacctcgctctgctaatcctgttaccagtggctgctgccagtggcgataagtcgtgtcttaccgggttgg
actcaagacgatagttaccggataaggcgcagcggtcgggctgaacggggggttcgtgcacacagcccagcttggagcgaacgacct
acaccgaactgagatacctacagcgtgagctatgagaaagcgccacgcttcccgaagggagaaaggcggacaggtatccggtaagcg
gcagggtcggaacaggagagcgcacgagggagcttccagggggaaacgcctggtatctttatagtcctgtcgggtttcgccacctct
gacttgagcgtcgatttttgtgatgctcgtcaggggggcggagcctatggaaaaacgccagcaacgcggcctttttacggttcctgg
cagatcctagatgtggcgcaacgatgccggcgacaagcaggagcgcaccgacttcttccgcatcaagtgttttggctctcaggccga
ggcccacggcaagtatttgggcaaggggtcgctggtattcgtgcagggcaagattcggaataccaagtacgagaaggacggccagac
ggtctacgggaccgacttcattgccgataaggtggattatctggacaccaaggcaccaggcgggtcaaatcaggaataagggcacat
tgccccggcgtgagtcggggcaatcccgcaaggagggtgaatgaatcggacgtttgaccggaaggcatacaggcaagaactgatcga
cgcggggttttccgccgaggatgccgaaaccatcgcaagccgcaccgtcatgcgtgcgccccgcgaaaccttccagtccgtcggctc
gatggtccagcaagctacggccaagatcgagcgcgacagcgtgcaactggctccccctgccctgcccgcgccatcggccgccgtgga
gcgttcgcgtcgtctcgaacaggaggcggcaggtttggcgaagtcgatgaccatcgacacgcgaggaactatgacgaccaagaagcg
aaaaaccgccggcgaggacctggcaaaacaggtcagcgaggccaagcaggccgcgttgctgaaacacacgaagcagcagatcaagga
aatgcagctttccttgttcgatattgcgccgtggccggacacgatgcgagcgatgccaaacgacacggcccgctctgccctgttcac
cacgcgcaacaagaaaatcccgcgcgaggcgctgcaaaacaaggtcattttccacgtcaacaaggacgtgaagatcacctacaccgg
cgtcgagctgcgggccgacgatgacgaactggtgtggcagcaggtgttggagtacgcgaagcgcacccctatcggcgagccgatcac
cttcacgttctacgagctttgccaggacctgggctggtcgatcaatggccggtattacacgaaggccgaggaatgcctgtcgcgcct
acaggcgacggcgatgggcttcacgtccgaccgcgttgggcacctggaatcggtgtcgctgctgcaccgcttccgcgtcctggaccg
tggcaagaaaacgtcccgttgccaggtcctgatcgacgaggaaatcgtcgtgctgtttgctggcgaccactacacgaaattcatatg
ggagaagtaccgcaagctgtcgccgacggcccgacggatgttcgactatttcagctcgcaccgggagccgtacccgctcaagctgga
aaccttccgcctcatgtgcggatcggattccacccgcgtgaagaagtggcgcgagcaggtcggcgaagcctgcgaagagttgcgagg
cagcggcctggtggaacacgcctgggtcaatgatgacctggtgcattgcaaacgctagggccttgtggggtcagttccggctggggg
ttcagcagccagcgcctgatctggggaaccctgtggttggcacatacaaatggacgaacggataaaccttttcacgcccttttaaat
atccgattattctaataaacgctcttttctcttaggtttacccgccaatatatcctgtcaaacactgatagtttaaactgaaggcgg
gaaacgacaatctgatctaagctaggcatgcctgcaggtcaacatggtggagcacgacacgcttgtctactccaaaaatatcaaaga
tacagtctcagaagaccaaagggcaattgagacttttcaacaaagggtaatatccggaaacctcctcggattccattgcccagctat
ctgtcactttattgtgaagatagtggaaaaggaaggtggctcctacaaatgccatcattgcgataaaggaaaggccatcgttgaaga
tgcctctgccgacagtggtcccaaagatggacccccacccacgaggagcatcgtggaaaaagaagacgttccaaccacgtcttcaaa
gcaagtggattgatgtgatatctccactgacgtaagggatgacgcacaatcccactatccttcgcaagacccttcctctatataagg
aagttcatttcatttggagaggagaaaactaaaccatacaccaccaacacaaccaaacccaccacgcccaattgttacacacccgct
tgaaaaagaaagtttaacaaatggccaaggtgcgcgaggtttaccaatcttttacagactccaccacaaaaactctcatccaagatg
aggcttatagaaacattcgccccatcatggaaaaacacaaactagctaacccttacgctcaaacggttgaagcggctaatgatctag
aggggttcggcatagccaccaatccctatagcattgaattgcatacacatgcagccgctaagaccatagagaataaacttctagagg
tgcttggttccatcctaccacaagaacctgttacatttatgtttcttaaacccagaaagctaaactacatgagaagaaacccgcgga
tcaaggacattttccaaaatgttgccattgaaccaagagacgtagccaggtaccccaaggaaacaataattgacaaactcacagaga
tcacaacggaaacagcatacattagtgacactctgcacttcttggatccgagctacatagtggagacattccaaaactgcccaaaat
tgcaaacattgtatgcgaccttagttctccccgttgaggcagcctttaaaatggaaagcactcacccgaacatatacagcctcaaat
acttcggagatggtttccagtatataccaggcaaccatggtggcggggcataccatcatgaattcgctcatctacaatggctcaaag
tgggaaagatcaagtggagggaccccaaggatagctttctcggacatctcaattacacgactgagcaggttgagatgcacacagtga
cagtacagttgcaggaatcgttcgcggcaaaccacttgtactgcatcaggagaggagacttgctcacaccggaggtgcgcactttcg
gccaacctgacaggtacgtgattccaccacagatcttcctcccaaaagttcacaactgcaagaagccgattctcaagaaaactatga
tgcagctcttcttgtatgttaggacagtcaaggtcgcaaaaaattgtgacatttttgccaaagtcagacaattaattaaatcatctg
acttggacaaatactctgctgtggaactggtttacttagtaagctacatggagttccttgccgatttacaagctaccacctgcttct
cagacacactttctggtggcttgctaacaaagacccttgcaccggtgagggcttggatacaagagaaaaagatgcagctgtttggtc
ttgaggactacgcgaagttagtcaaagcagttgatttccacccggtggatttttctttcaaagtggaaacttgggacttcagattcc
accccttgcaagcgtggaaagccttccgaccaagggaagtgtcggatgtagaggaaatggaaagtttgttctcagatggggacctgc
ttgattgcttcacaagaatgccagcttatgcggtaaacgcagaggaagatttagctgcaatcaggaaaacgcccgagatggatgtcg
gtcaagaagttaaagagcctgcaggagacagaaatcaatactcaaaccctgcagaaactttcctcaacaagctccacaggaaacaca
gtagggaggtgaaacaccaggccgcaaagaaagctaaacgcctagctgaaatccaggagtcaatgagagctgaaggtgatgccgaac
caaatgaaataagcgggacgatgggggcaatacccagcaacgccgaacttcctggcacgaatgatgccagacaagaactcacactcc
caaccactaaacctgtccctgcaaggtgggaagatgcttcattcacagattctagtgtggaagaggagcaggttaaactccttggaa
aagaaaccgttgaaacagcgacgcaacaagtcatcgaaggacttccttggaaacactggattcctcaattaaatgctgttggattca
aggcgctggaaattcagagggataggagtggaacaatgatcatgcccatcacagaaatggtgtccgggctggaaaaagaggacttcc
ctgaaggaactccaaaagagttggcacgagaattgttcgctatgaacagaagccctgccaccatccctttggacctgcttagagcca
gagactacggcagtgatgtaaagaacaagagaattggtgccatcacaaagacacaggcaacgagttggggcgaatacttgacaggaa
agatagaaagcttaactgagaggaaagttgcgacttgtgtcattcatggagctggaggttctggaaaaagtcatgccatccagaagg
cattgagagaaattggcaagggctcggacatcactgtagtcctgccgaccaatgaactgcggctagattggagtaagaaagtgccta
acactgagccctatatgttcaagacctctgaaaaggcgttaattgggggaacaggcagcatagtcatctttgacgattactcaaaac
ttcctcccggttacatagaagccttagtctgtttctactctaaaatcaagctaatcattctaacaggagatagcagacaaagcgtct
accatgaaactgctgaggacgcctccatcaggcatttgggaccagcaacagagtacttctcaaaatactgccgatactatctcaatg
ccacacaccgcaacaagaaagatcttgcgaacatgcttggtgtctacagtgagagaacgggagtcaccgaaatcagcatgagcgccg
agttcttagaaggaatcccaactttggtaccctcggatgagaagagaaagctgtacatgggcaccgggaggaatgacacgttcacat
acgctggatgccaggggctaactaagccgaaggtacaaatagtgttggaccacaacacccaagtgtgtagcgcgaatgtgatgtaca
cggcactttctagagccaccgataggattcacttcgtgaacacaagtgcaaattcctctgccttctgggaaaagttggacagcaccc
cttacctcaagactttcctatcagtggtgagagaacaagcactcagggagtacgagccggcagaggcagagccaattcaagagcctg
agccccagacacacatgtgtgtcgagaatgaggagtccgtgctagaagagtacaaagaggaactcttggaaaagtttgacagagaga
tccactctgaatcccatggtcattcaaactgtgtccaaactgaagacacaaccattcagttgttttcgcatcaacaagcaaaagatg
agactctcctctgggcgactatagatgcgcggctcaagaccagcaatcaagaaacaaacttccgagaattcctgagcaagaaggaca
ttggggacgttctgtttttaaactaccaaaaagctatgggtttacccaaagagcgtattcctttttcccaagaggtctgggaagctt
gtgcccacgaagtacaaagcaagtacctcagcaagtcaaagtgcaacttgatcaatgggactgtgagacagagcccagacttcgatg
aaaataagattatggtattcctcaagtcgcagtgggtcacaaaggtggaaaaactaggtctacccaagattaagccaggtcaaacca
tagcagccttttaccagcagactgtgatgctttttggaactatggctaggtacatgcgatggttcagacaggctttccagccaaaag
aagtcttcataaactgtgagacgacgccagatgacatgtctgcatgggccttgaacaactggaatttcagcagacctagcttggcta
atgactacacagctttcgaccagtctcaggatggagccatgttgcaatttgaggtgctcaaagccaaacaccactgcataccagagg
aaatcattcaggcatacatagatattaagactaatgcacagattttcctaggcacgttatcaattatgcgcctgactggtgaaggtc
ccacttttgatgcaaacactgagtgcaacatagcttacacccatacaaagtttgacatcccagccggaactgctcaagtttatgcag
gagacgactccgcactggactgtgttccagaagtgaagcatagtttccacaggcttgaggacaaattactcctaaagtcaaagcctg
taatcacgcagcaaaagaagggcagttggcctgagttttgtggttggctgatcacaccaaaaggggtgatgaaagacccaattaagc
tccatgttagcttaaaattggctgaagctaagggtgaactcaagaaatgtcaagattcctatgaaattgatctgagttatgcctatg
accacaaggactctctgcatgacttgttcgatgagaaacagtgtcaggcacacacactcacttgcagaacactaatcaagtcaggga
gaggcactgtctcactttcccgcctcagaaactttctttaaccgttaagttaccttagagatttgaataagatgtcagcaccagcta
gtacaacacagcccatagggtcaactacctcaactaccacaaaaactgcaggcgcaactcctgccacagcttcaggcctgttcacta
tcccggatggggatttctttagtacagcccgtgccatagtagccagcaatgctgtcgcaacaaatgaggacctcagcaagattgagg
ctatttggaaggacatgaaggtgcccacagacactatggcacaggctgcttgggacttagtcagacactgtgctgatgtaggatcat
ccgctcaaacagaaatgatagatacaggtccctattccaacggcatcagcagagctagactggcagcagcaattaaagaggtgtgca
cacttaggcaattttgcatgaagtatgccccagtggtatggaactggatgttaactaacaacagtccacctgctaactggcaagcac
aaggtttcaagcctgagcacaaattcgctgcattcgacttcttcaatggagtcaccaacccagctgccatcatgcccaaagaggggc
tcatccggccaccgtctgaagctgaaatgaatgctgcccaaactgctgcctttgtgaagattacaaaggccagggcacaatccaacg
actttgccagcctagatgcagctgtcactcgaggaaggatcaccggaacgaccacagcagaggcagtcgttactctgcctcctccat
aacagaaactttctttaaccgttaagttaccttagagatttgaataagatggatattctcatcagtagtttgaaaagtttaggttat
tctaggacttccaaatctttagattcaggacctttggtagtacatgcagtagccggagccggtaagtccacagccctaaggaagttg
atcctcagacacccaacattcaccgtgcatacactcggtgtccctgacaaggtgagtatcagaactagaggcatacagaagccagga
cctattcctgagggcaacttcgcaatcctcgatgagtatactttggacaacaccacaaggaactcataccaggcactttttgctgac
ccttatcaggcaccggagtttagcctagagccccacttctacttggaaacatcatttcgagttccgaggaaagtggcagatttgata
gctggctgtggcttcgatttcgagacgaactcaccggaagaagggcacttagagatcactggcatattcaaagggcccctactcgga
aaggtgatagccattgatgaggagtctgagacaacactgtccaggcatggtgttgagtttgttaagccctgccaagtgacgggactt
gagttcaaagtagtcactattgtgtctgccgcaccaatagaggaaattggccagtccacagctttctacaacgctatcaccaggtca
aagggattgacatatgtccgcgcagggccataggctgaccgctccggtcaattctgaaaaagtgtacatagtattaggtctatcatt
tgctttagtttcaattacctttctgctttctagaaatagcttaccccacgtcggtgacaacattcacagcttgccacacggaggagc
ttacagagacggcaccaaagcaatcttgtacaactccccaaatctagggtcacgagtgagtctacacaacggaaagaacgcagcatt
tgctgccgttttgctactgactttgctgatctatggaagtaaatacatatctcaacgcaatcatacttgtgcttgtggtaacaatca
tagcagtcattagcacttccttagtgaggactgaaccttgtgtcatcaagattactggggaatcaatcacagtgttggcttgcaaac
tagatgcagaaaccataagggccattgccgatctcaagccactctccgttgaacggttaagtttccattgatactcgaaagaggtca
gcaccagctagcaacaaacaagaacatgagagacctcgcgatttaaatcgatggtctcagatcggtcgtatcactggaacaacaacc
gctgaggctgttgtcactctaccaccaccataactacgtctacataaccgacgcctaccccagtttcatagtattttctggtttgat
tgtatgaataatataaataaaaaaaaaaaaaaaaaaaaaaaactagtgagct
SEQ ID NO: 24
>pNMD4300
aaactgaaggcgggaaacgacaatctgatctaagctaggcatgcctgcaggtcaacatggtggagcacgacacgcttgtctactcca
aaaatatcaaagatacagtctcagaagaccaaagggcaattgagacttttcaacaaagggtaatatccggaaacctcctcggattcc
attgcccagctatctgtcactttattgtgaagatagtggaaaaggaaggtggctcctacaaatgccatcattgcgataaaggaaagg
ccatcgttgaagatgcctctgccgacagtggtcccaaagatggacccccacccacgaggagcatcgtggaaaaagaagacgttccaa
ccacgtcttcaaagcaagtggattgatgtgatatctccactgacgtaagggatgacgcacaatcccactatccttcgcaagaccctt
cctctatataaggaagttcatttcatttggagaggagaaaactaaaccatacaccaccaacacaaccaaacccaccacgcccaattg
ttacacacccgcttgaaaaagaaagtttaacaaatggccaaggtgcgcgaggtttaccaatcttttacagactccaccacaaaaact
ctcatccaagatgaggcttatagaaacattcgccccatcatggaaaaacacaaactagctaacccttacgctcaaacggttgaagcg
gctaatgatctagaggggttcggcatagccaccaatccctatagcattgaattgcatacacatgcagccgctaagaccatagagaat
aaacttctagaggtgcttggttccatcctaccacaagaacctgttacatttatgtttcttaaacccagaaagctaaactacatgaga
agaaacccgcggatcaaggacattttccaaaatgttgccattgaaccaagagacgtagccaggtaccccaaggaaacaataattgac
aaactcacagagatcacaacggaaacagcatacattagtgacactctgcacttcttggatccgagctacatagtggagacattccaa
aactgcccaaaattgcaaacattgtatgcgaccttagttctccccgttgaggcagcctttaaaatggaaagcactcacccgaacata
tacagcctcaaatacttcggagatggtttccagtatataccaggcaaccatggtggcggggcataccatcatgaattcgctcatcta
caatggctcaaagtgggaaagatcaagtggagggaccccaaggatagctttctcggacatctcaattacacgactgagcaggttgag
atgcacacagtgacagtacagttgcaggaatcgttcgcggcaaaccacttgtactgcatcaggagaggagacttgctcacaccggag
gtgcgcactttcggccaacctgacaggtacgtgattccaccacagatcttcctcccaaaagttcacaactgcaagaagccgattctc
aagaaaactatgatgcagctcttcttgtatgttaggacagtcaaggtcgcaaaaaattgtgacatttttgccaaagtcagacaatta
attaaatcatctgacttggacaaatactctgctgtggaactggtttacttagtaagctacatggagttccttgccgatttacaagct
accacctgcttctcagacacactttctggtggcttgctaacaaagacccttgcaccggtgagggcttggatacaagagaaaaagatg
cagctgtttggtcttgaggactacgcgaagttagtcaaagcagttgatttccacccggtggatttttctttcaaagtggaaacttgg
gacttcagattccaccccttgcaagcgtggaaagccttccgaccaagggaagtgtcggatgtagaggaaatggaaagtttgttctca
gatggggacctgcttgattgcttcacaagaatgccagcttatgcggtaaacgcagaggaagatttagctgcaatcaggaaaacgccc
gagatggatgtcggtcaagaagttaaagagcctgcaggagacagaaatcaatactcaaaccctgcagaaactttcctcaacaagctc
cacaggaaacacagtagggaggtgaaacaccaggccgcaaagaaagctaaacgcctagctgaaatccaggagtcaatgagagctgaa
ggtgatgccgaaccaaatgaaataagcgggacgatgggggcaatacccagcaacgccgaacttcctggcacgaatgatgccagacaa
gaactcacactcccaaccactaaacctgtccctgcaaggtgggaagatgcttcattcacagattctagtgtggaagaggagcaggtt
aaactccttggaaaagaaaccgttgaaacagcgacgcaacaagtcatcgaaggacttccttggaaacactggattcctcaattaaat
gctgttggattcaaggcgctggaaattcagagggataggagtggaacaatgatcatgcccatcacagaaatggtgtccgggctggaa
aaagaggacttccctgaaggaactccaaaagagttggcacgagaattgttcgctatgaacagaagccctgccaccatccctttggac
ctgcttagagccagagactacggcagtgatgtaaagaacaagagaattggtgccatcacaaagacacaggcaacgagttggggcgaa
tacttgacaggaaagatagaaagcttaactgagaggaaagttgcgacttgtgtcattcatggagctggaggttctggaaaaagtcat
gccatccagaaggcattgagagaaattggcaagggctcggacatcactgtagtcctgccgaccaatgaactgcggctagattggagt
aagaaagtgcctaacactgagccctatatgttcaagacctctgaaaaggcgttaattgggggaacaggcagcatagtcatctttgac
gattactcaaaacttcctcccggttacatagaagccttagtctgtttctactctaaaatcaagctaatcattctaacaggagatagc
agacaaagcgtctaccatgaaactgctgaggacgcctccatcaggcatttgggaccagcaacagagtacttctcaaaatactgccga
tactatctcaatgccacacaccgcaacaagaaagatcttgcgaacatgcttggtgtctacagtgagagaacgggagtcaccgaaatc
agcatgagcgccgagttcttagaaggaatcccaactttggtaccctcggatgagaagagaaagctgtacatgggcaccgggaggaat
gacacgttcacatacgctggatgccaggggctaactaagccgaaggtacaaatagtgttggaccacaacacccaagtgtgtagcgcg
aatgtgatgtacacggcactttctagagccaccgataggattcacttcgtgaacacaagtgcaaattcctctgccttctgggaaaag
ttggacagcaccccttacctcaagactttcctatcagtggtgagagaacaagcactcagggagtacgagccggcagaggcagagcca
attcaagagcctgagccccagacacacatgtgtgtcgagaatgaggagtccgtgctagaagagtacaaagaggaactcttggaaaag
tttgacagagagatccactctgaatcccatggtcattcaaactgtgtccaaactgaagacacaaccattcagttgttttcgcatcaa
caagcaaaagatgagactctcctctgggcgactatagatgcgcggctcaagaccagcaatcaagaaacaaacttccgagaattcctg
agcaagaaggacattggggacgttctgtttttaaactaccaaaaagctatgggtttacccaaagagcgtattcctttttcccaagag
gtctgggaagcttgtgcccacgaagtacaaagcaagtacctcagcaagtcaaagtgcaacttgatcaatgggactgtgagacagagc
ccagacttcgatgaaaataagattatggtattcctcaagtcgcagtgggtcacaaaggtggaaaaactaggtctacccaagattaag
ccaggtcaaaccatagcagccttttaccagcagactgtgatgctttttggaactatggctaggtacatgcgatggttcagacaggct
ttccagccaaaagaagtcttcataaactgtgagacgacgccagatgacatgtctgcatgggccttgaacaactggaatttcagcaga
cctagcttggctaatgactacacagctttcgaccagtctcaggatggagccatgttgcaatttgaggtgctcaaagccaaacaccac
tgcataccagaggaaatcattcaggcatacatagatattaagactaatgcacagattttcctaggcacgttatcaattatgcgcctg
actggtgaaggtcccacttttgatgcaaacactgagtgcaacatagcttacacccatacaaagtttgacatcccagccggaactgct
caagtttatgcaggagacgactccgcactggactgtgttccagaagtgaagcatagtttccacaggcttgaggacaaattactccta
aagtcaaagcctgtaatcacgcagcaaaagaagggcagttggcctgagttttgtggttggctgatcacaccaaaaggggtgatgaaa
gacccaattaagctccatgttagcttaaaattggctgaagctaagggtgaactcaagaaatgtcaagattcctatgaaattgatctg
agttatgcctatgaccacaaggactctctgcatgacttgttcgatgagaaacagtgtcaggcacacacactcacttgcagaacacta
atcaagtcagggagaggcactgtctcactttcccgcctcagaaactttctttaaccgttaagttaccttagagatttgaataagatg
tcagcaccagctagtacaacacagcccatagggtcaactacctcaactaccacaaaaactgcaggcgcaactcctgccacagcttca
ggcctgttcactatcccggatggggatttctttagtacagcccgtgccatagtagccagcaatgctgtcgcaacaaatgaggacctc
agcaagattgaggctatttggaaggacatgaaggtgcccacagacactatggcacaggctgcttgggacttagtcagacactgtgct
gatgtaggatcatccgctcaaacagaaatgatagatacaggtccctattccaacggcatcagcagagctagactggcagcagcaatt
aaagaggtgtgcacacttaggcaattttgcatgaagtatgccccagtggtatggaactggatgttaactaacaacagtccacctgct
aactggcaagcacaaggtttcaagcctgagcacaaattcgctgcattcgacttcttcaatggagtcaccaacccagctgccatcatg
cccaaagaggggctcatccggccaccgtctgaagctgaaatgaatgctgcccaaactgctgcctttgtgaagattacaaaggccagg
gcacaatccaacgactttgccagcctagatgcagctgtcactcgaggaaggatcaccggaacgaccacagcagaggcagtcgttact
ctgcctcctccataacagaaactttctttaaccgttaagttaccttagagatttgaataagatggatattctcatcagtagtttgaa
aagtttaggttattctaggacttccaaatctttagattcaggacctttggtagtacatgcagtagccggagccggtaagtccacagc
cctaaggaagttgatcctcagacacccaacattcaccgtgcatacactcggtgtccctgacaaggtgagtatcagaactagaggcat
acagaagccaggacctattcctgagggcaacttcgcaatcctcgatgagtatactttggacaacaccacaaggaactcataccaggc
actttttgctgacccttatcaggcaccggagtttagcctagagccccacttctacttggaaacatcatttcgagttccgaggaaagt
ggcagatttgatagctggctgtggcttcgatttcgagacgaactcaccggaagaagggcacttagagatcactggcatattcaaagg
gcccctactcggaaaggtgatagccattgatgaggagtctgagacaacactgtccaggcatggtgttgagtttgttaagccctgcca
agtgacgggacttgagttcaaagtagtcactattgtgtctgccgcaccaatagaggaaattggccagtccacagctttctacaacgc
tatcaccaggtcaaagggattgacatatgtccgcgcagggccataggctgaccgctccggtcaattctgaaaaagtgtacatagtat
taggtctatcatttgctttagtttcaattacctttctgctttctagaaatagcttaccccacgtcggtgacaacattcacagcttgc
cacacggaggagcttacagagacggcaccaaagcaatcttgtacaactccccaaatctagggtcacgagtgagtctacacaacggaa
agaacgcagcatttgctgccgttttgctactgactttgctgatctatggaagtaaatacatatctcaacgcaatcatacttgtgctt
gtggtaacaatcatagcagtcattagcacttccttagtgaggactgaaccttgtgtcatcaagattactggggaatcaatcacagtg
ttggcttgcaaactagatgcagaaaccataagggccattgccgatctcaagccactctccgttgaacggttaagtttccattgatac
tcgaaagaggtcagcaccagctagcaacaaacaagaacatgagagacctcgcgatttaaatcgatggtctcagatcggtcgtatcac
tggaacaacaaccgctgaggctgttgtcactctaccaccaccataactacgtctacataaccgacgcctaccccagtttcatagtat
tttctggtttgattgtatgaataatataaataaaaaaaaaaaaaaaaaaaaaaaactagtgagctcttctgtcagcgggcccactgc
atccaccccagtacattaaaaacgtccgcaatgtgttattaagttgtctaagcgtcaatttgtttacaccacaatatatcctgccac
cagccagccaacagctccccgaccggcagctcggcacaaaatcaccactcgatacaggcagcccatcagtcagatcaggatctcctt
tgcgacgctcaccgggctggttgccctcgccgctgggctggcggccgtctatggccctgcaaacgcgccagaaacgccgtcgaagcc
gtgtgcgagacaccgcggccgccggcgttgtggatacctcgcggaaaacttggccctcactgacagatgaggggcggacgttgacac
ttgaggggccgactcacccggcgcggcgttgacagatgaggggcaggctcgatttcggccggcgacgtggagctggccagcctcgca
aatcggcgaaaacgcctgattttacgcgagtttcccacagatgatgtggacaagcctggggataagtgccctgcggtattgacactt
gaggggcgcgactactgacagatgaggggcgcgatccttgacacttgaggggcagagtgctgacagatgaggggcgcacctattgac
atttgaggggctgtccacaggcagaaaatccagcatttgcaagggtttccgcccgtttttcggccaccgctaacctgtcttttaacc
tgcttttaaaccaatatttataaaccttgtttttaaccagggctgcgccctgtgcgcgtgaccgcgcacgccgaaggggggtgcccc
cccttctcgaaccctcccggcccgctaacgcgggcctcccatccccccaggggctgcgcccctcggccgcgaacggcctcaccccaa
aaatggcagcctgtcgatcagatctggctcgcggcggacgcacgacgccggggcgagaccataggcgatctcctaaatcaatagtag
ctgtaacctcgaagcgtttcacttgtaacaacgattgagaatttttgtcataaaattgaaatacttggttcgcatttttgtcatccg
cggtcagccgcaattctgacgaactgcccatttagctggagatgattgtacatccttcacgtgaaaatttctcaagtgctgtgaaca
agggttcagattttagattgaaaggtgagccgttgaaacacgttcttcttgtcgatgacgacgtcgctatgcggcatcttattattg
aataccttacgatccacgccttcaaagtgaccgcggtagccgacagcacccagttcacaagagtactctcttccgcgacggtcgatg
tcgtggttgttgatctagatttaggtcgtgaagatgggctcgagatcgttcgtaatctggcggcaaagtctgatattccaatcataa
ttatcagtggcgaccgccttgaggagacggataaagttgttgcactcgagctaggagcaagtgattttatcgctaagccgttcagta
tcagagagtttctagcacgcattcgggttgccttgcgcgtgcgccccaacgttgtccgctccaaagaccgacggtctttttgtttta
ctgactggacacttaatctcaggcaacgtcgcttgatgtccgaagctggcggtgaggtgaaacttacggcaggtgagttcaatcttc
tcctcgcgtttttagagaaaccccgcgacgttctatcgcgcgagcaacttctcattgccagtcgagtacgcgacgaggaggtttatg
acaggagtatagatgttctcattttgaggctgcgccgcaaacttgaggcggatccgtcaagccctcaactgataaaaacagcaagag
gtgccggttatttctttgacgcggacgtgcaggtttcgcacggggggacgatggcagcctaagatcgacaggctggccaattcgtgc
gcggaacccctatttgtttatttttctaaatacattcaaatatgtatccgctcatgagacaataaccctgataaatgcttcaataat
attgaaaaaggaagagtatggctaaaatgagaatatcaccggaattgaaaaaactgatcgaaaaataccgctgcgtaaaagatacgg
aaggaatgtctcctgctaaggtatataagctggtgggagaaaatgaaaacctatatttaaaaatgacggacagccggtataaaggga
ccacctatgatgtggaacgggaaaaggacatgatgctatggctggaaggaaagctgcctgttccaaaggtcctgcactttgaacggc
atgatggctggagcaatctgctcatgagtgaggccgatggcgtcctttgctcggaagagtatgaagatgaacaaagccctgaaaaga
ttatcgagctgtatgcggagtgcatcaggctctttcactccatcgacatatcggattgtccctatacgaatagcttagacagccgct
tagccgaattggattacttactgaataacgatctggccgatgtggattgcgaaaactgggaagaagacactccatttaaagatccgc
gcgagctgtatgattttttaaagacggaaaagcccgaagaggaacttgtcttttcccacggcgacctgggagacagcaacatctttg
tgaaagatggcaaagtaagtggctttattgatcttgggagaagcggcagggcggacaagtggtatgacattgccttctgcgtccggt
cgatcagggaggatatcggggaagaacagtatgtcgagctattttttgacttactggggatcaagcctgattgggagaaaataaaat
attatattttactggatgaattgttttagctgtcagaccaagtttactcatatatactttagattgatttaaaacttcatttttaat
ttaaaaggatctaggtgaagatcdttttgataatctcatgaccaaaatcccttaacgtgagttttcgttccactgagcgtcagaccc
cgtagaaaagatcaaaggatcttcttgagatcctttttttctgcgcgtaatctgctgcttgcaaacaaaaaaaccaccgctaccagc
ggtggtttgtttgccggatcaagagctaccaactctttttccgaaggtaactggcttcagcagagcgcagataccaaatactgtcct
tctagtgtagccgtagttaggccaccacttcaagaactctgtagcaccgcctacatacctcgctctgctaatcctgttaccagtggc
tgctgccagtggcgataagtcgtgtcttaccgggttggactcaagacgatagttaccggataaggcgcagcggtcgggctgaacggg
gggttcgtgcacacagcccagcttggagcgaacgacctacaccgaactgagatacctacagcgtgagctatgagaaagcgccacgct
tcccgaagggagaaaggcggacaggtatccggtaagcggcagggtcggaacaggagagcgcacgagggagcttccagggggaaacgc
ctggtatctttatagtcctgtcgggtttcgccacctctgacttgagcgtcgatttttgtgatgctcgtcaggggggcggagcctatg
gaaaaacgccagcaacgcggcctttttacggttcctggcagatcctagatgtggcgcaacgatgccggcgacaagcaggagcgcacc
gacttcttccgcatcaagtgttttggctctcaggccgaggcccacggcaagtatttgggcaaggggtcgctggtattcgtgcagggc
aagattcggaataccaagtacgagaaggacggccagacggtctacgggaccgacttcattgccgataaggtggattatctggacacc
aaggcaccaggcgggtcaaatcaggaataagggcacattgccccggcgtgagtcggggcaatcccgcaaggagggtgaatgaatcgg
acgtttgaccggaaggcatacaggcaagaactgatcgacgcggggttttccgccgaggatgccgaaaccatcgcaagccgcaccgtc
atgcgtgcgccccgcgaaaccttccagtccgtcggctcgatggtccagcaagctacggccaagatcgagcgcgacagcgtgcaactg
gctccccctgccctgcccgcgccatcggccgccgtggagcgttcgcgtcgtctcgaacaggaggcggcaggtttggcgaagtcgatg
accatcgacacgcgaggaactatgacgaccaagaagcgaaaaaccgccggcgaggacctggcaaaacaggtcagcgaggccaagcag
gccgcgttgctgaaacacacgaagcagcagatcaaggaaatgcagctttccttgttcgatattgcgccgtggccggacacgatgcga
gcgatgccaaacgacacggcccgctctgccctgttcaccacgcgcaacaagaaaatcccgcgcgaggcgctgcaaaacaaggtcatt
ttccacgtcaacaaggacgtgaagatcacctacaccggcgtcgagctgcgggccgacgatgacgaactggtgtggcagcaggtgttg
gagtacgcgaagcgcacccctatcggcgagccgatcaccttcacgttctacgagctttgccaggacctgggctggtcgatcaatggc
cggtattacacgaaggccgaggaatgcctgtcgcgcctacaggcgacggcgatgggcttcacgtccgaccgcgttgggcacctggaa
tcggtgtcgctgctgcaccgcttccgcgtcctggaccgtggcaagaaaacgtcccgttgccaggtcctgatcgacgaggaaatcgtc
gtgctgtttgctggcgaccactacacgaaattcatatgggagaagtaccgcaagctgtcgccgacggcccgacggatgttcgactat
ttcagctcgcaccgggagccgtacccgctcaagctggaaaccttccgcctcatgtgcggatcggattccacccgcgtgaagaagtgg
cgcgagcaggtcggcgaagcctgcgaagagttgcgaggcagcggcctggtggaacacgcctgggtcaatgatgacctggtgcattgc
aaacgctagggccttgtggggtcagttccggctgggggttcagcagccagcgcctgatctggggaaccctgtggttggcacatacaa
atggacgaacggataaaccttttcacgcccttttaaatatccgattattctaataaacgctcttttctcttaggtttacccgccaat
atatcctgtcaaacactgatagttt
SEQ ID NO: 25: tttgaagacatctcaacgcaatcatacttgtgc
SEQ ID NO: 26: tttgaagacttctcggttatgtagacgtagttatggtg
SEQ ID NO: 27: GGTCTCNNNNN
SEQ ID NO: 28: AGGTRAG/GCAGGT
SEQ ID NO: 29: PVX 25K nucleotide sequence
atggatattc tcatcagtag tttgaaaagt ttaggttatt ctaggacttc caaatcttta gattcaggac ctttggtagt
acatgcagta gccggagccg gtaagtccac agccctaagg aagttgatcc tcagacaccc aacattcacc gtgcatacac
tcggtgtccc tgacaaggtg agtatcagaa ctagaggcat acagaagcca ggacctattc ctgagggcaa cttcgcaatc
ctcgatgagt atactttgga caacaccaca aggaactcat accaggcact ttttgctgac ccttatcagg caccggagtt
tagcctagag ccccacttct acttggaaac atcatttcga gttccgagga aagtggcaga tttgatagct ggctgtggct
tcgatttcga gacgaactca ccggaagaag ggcacttaga gatcactggc atattcaaag ggcccctact cggaaaggtg
atagccattg atgaggagtc tgagacaaca ctgtccaggc atggtgttga gtttgttaag ccctgccaag tgacgggact
tgagttcaaa gtagtcacta ttgtgtctgc cgcaccaata gaggaaattg gccagtccac agctttctac aacgctatca
ccaggtcaaa gggattgaca tatgtccgcg cagggccata g
SEQ ID NO: 30: PVX 25K protein sequence
MDILISSLKSLGYSRTSKSL DSGPLVVHAVAGAGKSTALR KLILRHPTFTVHTLGVPDKV SIRTRGIQKPGPIPEGNFAI
LDEYTLDNTTRNSYQALFAD PYQAPEFSLEPHFYLETSFR VPRKVADLIAGCGFDFETNS PEEGHLEITGIFKGPLLGKV
IAIDEESETTLSRHGVEFVK PCQVTGLEFKVVTIVSAAPI EEIGQSTAFYNAITRSKGLT YVRAGP
SEQ ID NO: 31: PVX 12K nucleotide sequence
atgtccgcgc agggccatag gctgaccgct ccggtcaatt ctgaaaaagt gtacatagta ttaggtctat catttgcttt
agtttcaatt acctttctgc tttctagaaa tagcttaccc cacgtcggtg acaacattca cagcttgcca cacggaggag
cttacagaga cggcaccaaa gcaatcttgt acaactcccc aaatctaggg tcacgagtga gtctacacaa cggaaagaac
gcagcatttg ctgccgtttt gctactgact ttgctgatct atggaagtaa atacatatct caacgcaatc atacttgtgc
ttgtggtaac aatcatagca gtcat
SEQ ID NO: 32: PVX 12K protein sequence
MSAQGHRLTAPVNSEKVYIVLGLSFALVSITFLLSRNSLPHVGDNIHSLPHGGAYRDGTKAILYNSPNLGSRVSLHNGKNAAFAAVL
LLTLLIYGSKYISQRNHTCACGNNHSSH
SEQ ID NO: 33: PVX 8K nucleotide sequence
atggaagtaa atacatatct caacgcaatc atacttgtgc ttgtggtaac aatcatagca gtcattagca cttccttagt
gaggactgaa ccttgtgtca tcaagattac tggggaatca atcacagtgt tggcttgcaa actagatgca gaaaccataa
gggccattgc cgatctcaag ccactctccg ttgaacggtt aagtttccat
SEQ ID NO: 34: PVX 8K protein sequence
MEVNTYLNAIILVLVVTIIAVISTSLVRTEPCVIKITGESITVLACKLDAETIRAIADLKPLSVERLSFH
SEQ ID NO: 35: PVX coat protein coding sequence
atgtcagcac cagctagcac aacacagccc atagggtcaa ctacctcaac taccacaaaa actgcaggcg caactcctgc
cacagcttca ggcctgttca ctatcccgga tggggatttc tttagtacag cccgtgccat agtagccagc aatgctgtcg
caacaaatga ggacctcagc aagattgagg ctatttggaa ggacatgaag gtgcccacag acactatggc acaggctgct
tgggacttag tcagacactg tgctgatgta ggatcatccg ctcaaacaga aatgatagat acaggtccct attccaacgg
catcagcaga gctagactgg cagcagcaat taaagaggtg tgcacactta ggcaattttg catgaagtat gccccagtgg
tatggaactg gatgttaact aacaacagtc cacctgctaa ctggcaagca caaggtttca agcctgagca caaattcgct
gcattcgact tcttcaatgg agtcaccaac ccagctgcca tcatgcccaa agaggggctc atccggccac cgtctgaagc
tgaaatgaat gctgcccaaa ctgctgcctt tgtgaagatt acaaaggcca gggcacaatc caacgacttt gccagcctag
atgcagctgt cactcgaggt cgtatcactg gaacaacaac cgctgaggct gttgtcactc taccaccacc ataa
SEQ ID NO: 36: PVX coat protein
MSAPASTTQPIGSTTSTTTKTAGATPATASGLFTIPDGDFFSTARAIVASNAVATNEDLSKIEAIWKDMYVPTDTMAQAAWDLVRHC
ADVGSSAQTEMIDTGPYSNGISRARLAAAIKEVCTLRQFCMKYAPVVWNWMLTNNSPPANWQAQGFKPEHKFAAFDFFNGVTNPAAI
MPKEGLIRPPSEAEMNAAQTAAFVKITKARAQSNDFASLDAAVTRGRITGTTTAEAVVTLPPP
SEQ ID NO: 37: PVX RdRp coding sequence
atggccaagg tgcgcgaggt ttaccaatct tttacagact ccaccacaaa aactctcatc caagatgagg cttatagaaa
cattcgcccc atcatggaaa aacacaaact agctaaccct tacgctcaaa cggttgaagc ggctaatgat ctagaggggt
tcggcatagc caccaatccc tatagcattg aattgcatac acatgcagcc gctaagacca tagagaataa acttctagag
gtgcttggtt ccatcctacc acaagaacct gttacattta tgtttcttaa acccagaaag ctaaactaca tgagaagaaa
cccgcggatc aaggacattt tccaaaatgt tgccattgaa ccaagagacg tagccaggta ccccaaggaa acaataattg
acaaactcac agagatcaca acggaaacag catacattag tgacactctg cacttcttgg atccgagcta catagtggag
acattccaaa actgcccaaa attgcaaaca ttgtatgcga ccttagttct ccccgttgag gcagccttta aaatggaaag
cactcacccg aacatataca gcctcaaata cttcggagat ggtttccagt atataccagg caaccatggt ggcggggcat
accatcatga attcgctcat ctacaatggc tcaaagtggg aaagatcaag tggagggacc ccaaggatag ctttctcgga
catctcaatt acacgactga gcaggttgag atgcacacag tgacagtaca gttgcaggaa tcgttcgcgg caaaccactt
gtactgcatc aggagaggag acttgctcac accggaggtg cgcactttcg gccaacctga caggtacgtg attccaccac
agatcttcct cccaaaagtt cacaactgca agaagccgat tctcaagaaa actatgatgc agctcttctt gtatgttagg
acagtcaagg tcgcaaaaaa ttgtgacatt tttgccaaag tcagacaatt aattaaatca tctgacttgg acaaatactc
tgctgtggaa ctggtttact tagtaagcta catggagttc cttgccgatt tacaagctac cacctgcttc tcagacacac
tttctggtgg cttgctaaca aagacccttg caccggtgag ggcttggata caagagaaaa agatgcagct gtttggtctt
gaggactacg cgaagttagt caaagcagtt gatttccacc cggtggattt ttctttcaaa gtggaaactt gggacttcag
attccacccc ttgcaagcgt ggaaagcctt ccgaccaagg gaagtgtcgg atgtagagga aatggaaagt ttgttctcag
atggggacct gcttgattgc ttcacaagaa tgccagctta tgcggtaaac gcagaggaag atttagctgc aatcaggaaa
acgcccgaga tggatgtcgg tcaagaagtt aaagagcctg caggagacag aaatcaatac tcaaaccctg cagaaacttt
cctcaacaag ctccacagga aacacagtag ggaggtgaaa caccaggccg caaagaaagc taaacgccta gctgaaatcc
aggagtcaat gagagctgaa ggtgatgccg aaccaaatga aataagcggg acgatggggg caatacccag caacgccgaa
cttcctggca cgaatgatgc cagacaagaa ctcacactcc caaccactaa acctgtccct gcaaggtggg aagatgcttc
attcacagat tctagtgtgg aagaggagca ggttaaactc cttggaaaag aaaccgttga aacagcgacg caacaagtca
tcgaaggact tccttggaaa cactggattc ctcaattaaa tgctgttgga ttcaaggcgc tggaaattca gagggatagg
agtggaacaa tgatcatgcc catcacagaa atggtgtccg ggctggaaaa agaggacttc cctgaaggaa ctccaaaaga
gttggcacga gaattgttcg ctatgaacag aagccctgcc accatccctt tggacctgct tagagccaga gactacggca
gtgatgtaaa gaacaagaga attggtgcca tcacaaagac acaggcaacg agttggggcg aatacttgac aggaaagata
gaaagcttaa ctgagaggaa agttgcgact tgtgtcattc atggagctgg aggttctgga aaaagtcatg ccatccagaa
ggcattgaga gaaattggca agggctcgga catcactgta gtcctgccga ccaatgaact gcggctagat tggagtaaga
aagtgcctaa cactgagccc tatatgttca agacctctga aaaggcgtta attgggggaa caggcagcat agtcatcttt
gacgattact caaaacttcc tcccggttac atagaagcct tagtctgttt ctactctaaa atcaagctaa tcattctaac
aggagatagc agacaaagcg tctaccatga aactgctgag gacgcctcca tcaggcattt gggaccagca acagagtact
tctcaaaata ctgccgatac tatctcaatg ccacacaccg caacaagaaa gatcttgcga acatgcttgg tgtctacagt
gagagaacgg gagtcaccga aatcagcatg agcgccgagt tcttagaagg aatcccaact ttggtaccct cggatgagaa
gagaaagctg tacatgggca ccgggaggaa tgacacgttc acatacgctg gatgccaggg gctaactaag ccgaaggtac
aaatagtgtt ggaccacaac acccaagtgt gtagcgcgaa tgtgatgtac acggcacttt ctagagccac cgataggatt
cacttcgtga acacaagtgc aaattcctct gccttctggg aaaagttgga cagcacccct tacctcaaga ctttcctatc
agtggtgaga gaacaagcac tcagggagta cgagccggca gaggcagagc caattcaaga gcctgagccc cagacacaca
tgtgtgtcga gaatgaggag tccgtgctag aagagtacaa agaggaactc ttggaaaagt ttgacagaga gatccactct
gaatcccatg gtcattcaaa ctgtgtccaa actgaagaca caaccattca gttgttttcg catcaacaag caaaagatga
gactctcctc tgggcgacta tagatgcgcg gctcaagacc agcaatcaag aaacaaactt ccgagaattc ctgagcaaga
aggacattgg ggacgttctg tttttaaact accaaaaagc tatgggttta cccaaagagc gtattccttt ttcccaagag
gtctgggaag cttgtgccca cgaagtacaa agcaagtacc tcagcaagtc aaagtgcaac ttgatcaatg ggactgtgag
acagagccca gacttcgatg aaaataagat tatggtattc ctcaagtcgc agtgggtcac aaaggtggaa aaactaggtc
tacccaagat taagccaggt caaaccatag cagcctttta ccagcagact gtgatgcttt ttggaactat ggctaggtac
atgcgatggt tcagacaggc tttccagcca aaagaagtct tcataaactg tgagacgacg ccagatgaca tgtctgcatg
ggccttgaac aactggaatt tcagcagacc tagcttggct aatgactaca cagctttcga ccagtctcag gatggagcca
tgttgcaatt tgaggtgctc aaagccaaac accactgcat accagaggaa atcattcagg catacataga tattaagact
aatgcacaga ttttcctagg cacgttatca attatgcgcc tgactggtga aggtcccact tttgatgcaa acactgagtg
caacatagct tacacccata caaagtttga catcccagcc ggaactgctc aagtttatgc aggagacgac tccgcactgg
actgtgttcc agaagtgaag catagtttcc acaggcttga ggacaaatta ctcctaaagt caaagcctgt aatcacgcag
caaaagaagg gcagttggcc tgagttttgt ggttggctga tcacaccaaa aggggtgatg aaagacccaa ttaagctcca
tgttagctta aaattggctg aagctaaggg tgaactcaag aaatgtcaag attcctatga aattgatctg agttatgcct
atgaccacaa ggactctctg catgacttgt tcgatgagaa acagtgtcag gcacacacac tcacttgcag aacactaatc
aagtcaggga gaggcactgt ctcactttcc cgcctcagaa actttcttta a
SEQ ID NO: 38
>sGFP with Nicotiana tabacum codon usage
atggtctcaaaaggagaagagttgtttacaggtgttgttcccattctagtggagttagatggcgatgtgaatggacataagttttcc
gttagtggtgaaggcgaaggagatgcaacatatgggaaattgacactcaagtttatctgtactacagggaaattaccagttccatgg
cctacattggtcactaccttttcttatggtgtgcaatgctttagcagatatccagatcacatgaagcaacatgacttctttaagtct
gctatgcctgaaggctatgttcaggagagaaccattttcttcaaggatgatggtaactataaaacgagagctgaggtaaagtttgaa
ggagacactcttgttaatcgaatagaactgaaaggaattgacttcaaggaagatggcaatatacttggtcacaaacttgagtacaac
tacaatagtcacaatgtgtacattatggcggacaaacagaagaatgggatcaaagtcaacttcaagataaggcacaatatcgaagat
ggatctgtgcaacttgcagaccattaccaacagaacactccgattggagatggacctgtactattgccagataaccattatctctct
actcaatcagccttgtccaaagaccctaatgagaaacgtgatcatatggtactgttagagtttgttaccgcagctggtattactcat
ggtatggatgaactttacaagtaa

The content of European patent application No. 17 191 524.2, filed on Sep. 18, 2017, the priority of which is claimed by the present patent application, is incorporated by reference in its entirety including all claims, description, all drawings and sequences.

1. A method of producing a potexviral vector for expressing a protein of interest in a plant, comprising
producing a second heterologous nucleic acid sequence comprising a second ORF encoding said protein of interest and having, in the second ORF, an increased GC-content compared to a first ORF encoding said protein in a first heterologous nucleic acid sequence, and
providing said potexviral vector comprising the following segments: (i) a nucleic acid sequence encoding a potexviral RNA-dependent RNA polymerase, (ii) a nucleic acid sequence comprising or encoding a potexviral triple-gene block, and (iii) said second heterologous nucleic acid sequence or a portion thereof comprising said second ORF.

2. A method of improving the capability for long-distance movement in a plant of a potexviral replicon encoding a protein of interest to be expressed in said plant, comprising
producing a second heterologous nucleic acid sequence comprising a second ORF encoding said protein of interest and having, in the second ORF, an increased GC-content compared to a first ORF encoding said protein of interest in a first heterologous nucleic acid sequence, and
providing said potexviral replicon, or a potexviral vector comprising or encoding said potexviral replicon, said potexviral replicon comprising
(a) the following segments: (i) a nucleic acid sequence encoding a potexviral RNA-dependent RNA polymerase, (ii) a nucleic acid sequence comprising (ii-a) a potexviral triple-gene block and (ii-b) a nucleic acid sequence encoding a potexviral coat protein or a nucleic acid sequence encoding a tobamoviral movement protein, and (iii) said second ORF, said second heterologous nucleic acid sequence or a portion thereof, said portion comprising said second ORF;
(b) the following segments: (i) a nucleic acid sequence encoding a potexviral RNA-dependent RNA polymerase, (ii) a nucleic acid sequence comprising a potexviral triple-gene block, and (iii) said second heteroloqous nucleic acid sequence or a portion thereof comprising said second ORF; or
(c) the following segments: (i) a nucleic acid sequence encoding a potexviral RNA-dependent RNA polymerase, (ii) a nucleic acid sequence comprising (ii-a) a potexviral triple-gene block and (ii-b) a nucleic acid sequence encoding a potexviral coat protein or a nucleic acid sequence encoding a tobamoviral movement protein, and (iii) said second ORF, said second heteroloqous nucleic acid sequence or a portion thereof, said portion comprising said second ORF.

3-4. (canceled)

5. The method according to any claim 2, wherein said step of providing a potexviral vector or potexviral replicon comprises inserting said second heterologous nucleic acid sequence, or a portion thereof comprising said second ORF, into a nucleic acid comprising (i) a nucleic acid sequence encoding a potexviral RNA-dependent RNA polymerase and (ii) a nucleic acid sequence encoding a potexviral triple-gene block to produce the potexviral vector or the potexviral replicon comprising the second heterologous nucleic acid sequence or a portion thereof comprising said second ORF.

6. A process of expressing a protein of interest in a plant or in plant tissue, comprising producing a potexviral vector according to the method of claim 2 and providing the produced potexviral vector to at least a part of said plant.

7. The method or process according to claim 2, wherein said plant is selected from Nicotiana species such as Nicotiana benthamiana and Nicotiana tabacum, tomato, potato, pepper, eggplant, soybean, Petunia hybrida, Brassica napus, Brassica campestris, Brassica juncea, cress, arugula, mustard, strawberry, spinach, Chenopodium capitatum, alfalfa, lettuce, sunflower, potato, cucumber, corn, wheat, and rice.

8. The method or process according to claim 2, wherein said (ii) nucleic acid sequence comprising or encoding a potexviral triple-gene block further comprises a nucleic acid sequence encoding a potexviral coat protein or a nucleic acid sequence encoding a tobamoviral movement protein.

9. A method of improving the capability for long-distance movement in a plant of a potexviral replicon encoding a protein of interest to be expressed in said plant, comprising
increasing the GC-content of a first ORF encoding said protein in a first heterologous nucleic acid sequence, thereby obtaining a second heterologous nucleic acid sequence comprising a second ORF, said second ORF encoding said protein and having an increased GC-content, and
inserting said second heterologous nucleic acid sequence, or a portion thereof containing said second ORF, into a nucleic acid comprising (i) a nucleic acid sequence encoding a potexviral RNA-dependent RNA polymerase and (ii) a nucleic acid sequence comprising or encoding a potexviral triple-gene block to produce a potexviral vector comprising or encoding said potexviral replicon, said potexviral vector comprising the second heterologous nucleic acid sequence or a portion thereof comprising said second ORF.

10. A potexviral vector obtained or obtainable by the method of claim 1, wherein the protein of interest is not a plant viral protein, or wherein the protein of interest is a protein that is heterologous to plant viruses.

11. A nucleic acid comprising the following segments: (i) a nucleic acid sequence encoding a potexviral RNA-dependent RNA polymerase, (ii) nucleic acid sequence comprising or encoding a potexviral triple-gene block, and (iii) a heterologous nucleic acid sequence comprising an ORF encoding a protein of interest, wherein:
(a) said ORF consists of at least 200 and at most 400 nucleotides and has a GC-content of at least 50%; or
said ORF consists of at least 401 and at most 800 nucleotides has a GC-content of at least 55%; and/or
said ORF consists of at least 801 nucleotides and has a GC-content of at least 58%;
(b) said ORF consists of at least 100 and at most 500 nucleotides and has a GC-content of at least 50%; or
said ORF consists of at least 501 and at most 1000 nucleotides has a GC-content of at least 55%; and/or
said ORF consists of at least 1001 nucleotides and has a GC-content of at least 58%; and
wherein the protein of interest is not a plant viral protein or wherein the protein of interest is a protein that is heterologous to plant viruses.

12. (canceled)

13. The nucleic acid according to claim 11, said nucleic acid further comprising, preferably in the nucleic acid sequence of (ii), a nucleic acid sequence encoding a potexviral coat protein or a nucleic acid sequence encoding a tobamoviral movement protein.

14. A combination or kit comprising a first and a second nucleic acid, said first nucleic acid comprising segments (i) and (ii) as defined in claim 11, said second nucleic acid comprising segment (iii) as defined in claim 11.

15. The combination or kit according to claim 14, wherein said first nucleic acid has, downstream of segment (ii) a first site-specific recombination site recognizable by a site-specific recombinase, and said second nucleic add has, upstream of segment (iii), a second site-specific recombination site recognizable by said site-specific recombinase for allowing site-specific recombination between said first and said second site-specific recombination site and formation of a nucleic acid comprising the following segments: a nucleic acid sequence encoding a potexviral RNA-dependent RNA polymerase, (ii) nucleic acid sequence comprising or encoding a potexviral triple-gene block, and (iii) a heterologous nucleic add sequence comprising an ORF encoding a protein of interest, wherein
said ORF consists of at least 200 and at most 400 nucleotides and has a GC-content of at least 50%; or
said ORF consists of at east 401 and at most 800 nucleotides has a GC-content of at east 55%; and/or
said ORF consists of at least 801 nucleotides and has a GC-content of at least 58%,
wherein the protein of interest is not a plant viral protein or wherein the protein of interest is a protein that is heterologous to plant viruses.

16. A process of expressing a nucleic acid sequence of interest in a plant or in plant tissue, comprising providing the plant or plant tissue with said nucleic acid of claim 11.

17. Use of a nucleic acid as defined in claim 11, for expressing a protein encoded by said heterologous nucleic acid and for achieving improved long-distance movement of a potexviral vector in a plant.


SEQUENCE LISTING (38 sequences)

SEQ ID NO: 1 - DNA, 528 bp, Artificial Sequence
  AtFT, one nucleotide exchange
atgtctataaatataagggaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttag

SEQ ID NO: 2 - DNA, 648 bp, Capsicum annuum
atgaacatctttagaagctattattcggacccacttactgaatcttcatcatctttttctgatagtagcatttactcccctaatagagctattttttctgatgaggaagttatattagcatcaaataacccgaaaaagccagctgggaggaagaagtttcgagaaactcgacatccagtatacaggggagttaggaagaggaattcaggcaaatgggtttgtgaagtcagagaacccaataagaaatcaagaatttggcttggtacttttcctacagctgaaatggctgctagagctcatgacgtggcggctatagcattaagaggtcgttctgcttgtttgaactttgctgattctgcttggaggttgcctgttccggcttcctctgacactaaagatattcaaaaggcggccgctgaggccgcggaagccctccgaccattgaagttggaaggaatttcaaaagaatcatctagcagtactccagagagtatgttctttatggatgaggaagcgctcttctgcatgccgggattacttacgaatatggctgaagggctaatgttaccaccacctcaatgtgcagaaattggagatcatgtggaaactgctgatgcggatacccctttatggagctattccatttaa

SEQ ID NO: 3 - DNA, 663 bp, Antirrhinum majus
atggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaa

SEQ ID NO: 4 - DNA, 666 bp, Glycine max
atggaaactcaacaccaacaacccaccatcaagtctaggttcagacgcatctgtgtctactgtggtagcagccctggcaaaaaccccagctaccagctcgctgctattcaactcggaaaacaactggtggagaggaacattgacttggtttatggaggaggaagcatagggttgatgggtctaatctcacaagttgtgtatgatggtggacgccacgtgttaggggtgattccagagacacttaatgcaagagagataactggagagagtgttggagaagtgagagctgtatcgggcatgcaccaacgcaaagccgaaatggcccgacaagccgatgcatttattgcactgccaggtggatatggcacccttgaagaactactggaaattatcacctgggctcaactaggcatccatgataaaccggtggggttgttgaacgtggatgggtactacaactcgctgctggcattcatggacaaagctgtggacgaaggtttcgtaacaccagctgcccgtcacattattgtttctgcccacactgcccaagaactcatgtgcaaacttgaggaatatgtccccgagcactgtggcgtggcccccaagctaagttgggagatggagcaacagttagttaacactgcaaagtcagatatttcccgttga

SEQ ID NO: 5 - DNA, 678 bp, Lycopersicon esculentum
atggaaaacaatcaccagacacaaattcagaccactaaaacatcaagattcaaacgcatatgtgttttttgtggaagcagtccaggcaaaaagccaagttatcaacttgctgctattcaacttggcaatcaactggttgaaaggaacatcgacttggtttatggaggtggcagtgtgggcttgatgggcctagtttctcaatcagtttttaatggtggccgccacgtgttaggggtgattcctaaaactcttatgccaagagagattactggagaaagtgttggagaagtaagagcagtgtctgggatgcatcaaagaaaagcagaaatggcaagacaagctgatgcattcatagccttaccaggtggctatgggacattggaagagctcctagaagtcatcacttgggctcaactaggcattcatgataaaccagtaggtttacttaatgtagatggctactataattcattattatcatttatagacaaagctgttgatgaaggctttgtcacaccctctgcccgtcacatcattatttctgccccaactgcccaagaactcatgtctaagcttgaggattatgtaccaaagcataatggggtggcaccaaaattgagttgggaaatggaacaacaacttggctacacaacaacaaaattggaaattgctcgttaa

SEQ ID NO: 6 - DNA, 720 bp, Artificial Sequence
  sGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaa

SEQ ID NO: 7 - DNA, 732 bp, Lycopersicon esculentum
atgctgccaagaagatatcctcagatggatgctaatcctagtaatgggggtgaaagggataatgctttgcgaggaattctgcaggacttatggccactggatgaaattgatccaagcactcaaaagttcccttgttgccttgtttggactcctctccctgtgatttcttggcttgcaccttttgttggacatgttggcatatgcagggaggatggtaccattgtggatttttctggagatagcatgattcattttggtcagctcttctatggaactgtagccaaatactatcaggtagacagacagcagtgctgttttgctcgcaactttggtggacacacatgccgtaagggttatgaacatgttgtatttgggacagcagtaagttgggatgatgctgttcagttgtttaggcgcacctttgagaacagaaacttcaaagttttcagttgcaacggccactcattcgctgctgattgcctgaacctgctatcatttagaggatcaatgcgctggaacatgattaatgttggagctcttataatgtttgagggaaagtgggtcagtcgctggtcaatgttacgatcatttctgcctttcattgggatactttgcttcggctatttaatgattggatggatgtttccaattggtctgctctcctttgttattgggacttttggatggtatgtcatgatctgttactgttgcaagattgaggatgacaattag

SEQ ID NO: 8 - DNA, 903 bp, Lycopersicon esculentum
atggctattatggatgaagctgctaatatggtttgtgtgccgttggattatagtagaaagaggaaatcaaggagtagaagggacagaacaaaaaatgtggaagagacactagctaaatggaaggagtataatgagaaactagacaatgaagggaaagggaagccagtgcgtaaagttcctgctaaaggttcaaagaaggggtgtatgagaggtaaagggggaccagaaaattggcggtgtaaatacagaggtgttagacagaggatatggggtaaatgggttgctgagattagggaacctaaaagaggtagtaggttatggttgggtacatttggtacagcaattgaagctgctttagcatatgatgatgctgcaagagctatgtatggtccttgtgcaaggcttaatttgccaaattacgcgtgtgattctgtttcctgggcaactacatctgcatctgcatctgcatctgattgcaccgttgcttctggtttcggcgaggtatgtccggttgatggtgctcttcatgaagctgacacaccattgagctcagtgaaagacgaagggaccgcgatggatattgttgaacctacgagtattgatgaagatacgcttaagtctggatgggattgtctagataaattaaatatggatgagatgtttgatgtagatgagctattggctatgttagattctactccagttttcaccaaggactacaattcagatggaaagcacaacaatatggtatcagattcgcaatgtcaggagccgaatgcagtggtagatcctatgactgttgactatggctttgattttctgaaaccaggcaggcaagaagatcttaatttcagttcggatgaccttgcattcatagacttggattctgaacttgtcgtttga

SEQ ID NO: 9 - DNA, 1059 bp, Lycopersicon esculentum
atgggaaaaagtttgaagcttcggttctccagagttattgcttctttcaattcgtgccgttcgaaaaacccttcttctcttccccaaaatcctaatttcttcccacataagctcactagtacaaaacacatttcccccgatttccctcttattgatcaaaatcaaaatcaaaatcaccgtaattacgtgccagaatccacgatgatctccgttgggtgttgtagatcagaattcaagtgggagaaagaagagaagtttcacgtggtttctagttccttcgtgtctgaagaagaagaatgtgaagaggagatcaatttggccttacgacctcctcttacacctccgcgattcagtagaattgttgttgagaagaagaagaagaaacaacagcgagttaaaaaaacgaaaacaaaaagtagaatcatccgaatgagtacttcctcagctgatgagtacagcgggatattaagcggtactaatactgattgggataataatgaagaggaaactgaatctttagtttcatcttccagaagctgttacgatttctcaagcgatgactcatctactgatttcaaccctcacttagaaaccatatgtgagaccactacaatgaggcgtcgtcacaagagaaatgccaacaccaagaggagatcaatcaagcaatccagaccaagtttttcctcttcaaaaggtagaagatcgtcggtttctacgtcatcagatagcgagctaccggcaaggttatcggtgtttaagaagctgataccgtgtagtgtggatgggaaagtgaaggagagtttcgcgatagtgaagaaatctcaggacccgtacgaagatttcaagagatcgatgatggaaatgattttagagaaggaaatgtttgagaagaatgagctggaacagcttttacaatgttttctgtcgttgaacggaaagcattatcatggagtgatagttgaggcgttctcagacatttgggagactttgtttttaggtaataatgatagagtaaggaggatgtcaattcatgatcccacacccacctactgtaggtag

SEQ ID NO: 10 - DNA, 1266 bp, Lycopersicon esculentum
atgggaaagcgaagaaactggtttacctttgtcaagagacttttcattcctgaaacagaatcaacagcagatcaaaagaaaccaaagagatggagatgttgttttctgagaaagttcaagttgaggaaatgtcctgctataacatcagcacctcagcaaacgttacctgaggcgaaaggaacacctcagcaaacgttaactgaggcgaaagaacagcaaagaaaacatgcttttgcagttgctatagcaacggcagcagctgctgaggctgctgtagctgctgctaatgctgctgctgatgttattcgtctaacagatgctccaagtgaattcaaaaggaaacgcaaacaagctgctattagaatccaaagtgcttatcgcgctcacctggcccagaaagcattaagggctctaaagggtgttgtgaagcttcaagcagtgattagaggtgaaattgtgagaggaagactcattgccaaactgaagttcatgttgccacttcatcaaaagtcaaaaacaagagttaatcaaattagagtccctacttttgaagatcatcatgacaagaaactcatcaatagtccaagggaaattatgaaagctaaagaactaaagcttaaatgcaagagccttagcacttggaatttcaacttagcttcagaacaagacagtgaagccttgtggtcaagaagagaagaagccattgacaaaagagagcatttgatgaaatactcgttttcacatcgggagagaagaaacgatcaaactctacaagacttactaaacagaaagcaaaacagaagaagctacaggattgaccagttagtagaacttgacgcaccaagaaaagcagggttgttagagaaattgagatcatttacagactcaaatgttcctctaactgatatggatggaatgacacagcttcaagtgagaaaaatgcatagatcagattgtatagaggacctacattctccttcttcacttccaagaagatcattttctaatgcaaaacgaaaatcaaacgttgatgataactcattaccaagttctcctatatttcctacttacatggcagccacagaatctgcaaaggcaaaaacaaggtcaaacagcacagcgaagcaacacctaaggttacacgagacattgtcaggtcaacattctccttataacctcaagatttcttcttggagattgtctaatggtgaaatgtatgacagcgccagaacaagcagaacttctagcagttatatgttaatatag

SEQ ID NO: 11 - DNA, 1812 bp, Artificial Sequence
  GUS, nucleotide exchanges G835C and G903A
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 12 - DNA, 2193 bp, Lycopersicon esculentum
atgtttaataaccaccagcacttgctcgatatatcgtcctcagctcaacgaacacctgataacgagttggatttcattcgtgatgaagagtttgatagcaactctggtgctgataacatggaagctcccaattcaggtgatgacgatcaagctgatccaaaccaacctccaaacaagaagaagcgttatcatcgccacactcagaatcagattcaggaaatggagtccttttacaaggaatgcaatcatccagatgacaagcaaaggaaggaattgggaagaagacttggtttggagccattacaagtgaaattttggttccagaacaagcgtactcagatgaaggctcaacatgagcgatgtgagaacacacagttgaggaatgaaaatgagaagcttcgcgctgagaacataaggtacaaagaagctttgagtaatgcagcatgcccaaattgtggagggccagcagctataggagagatgtcatttgatgagcatcagttgaggattgaaaatgctcgtcttagagatgagattgacaggataactggaatagctggaaagtatgttggtaaatcagcccttggatattctcatcaacttcctcttcctcagcccgaagctcctcgggttctggatcttgcttttgggcctcaatcgggcctgcttggagaaatgtacgctgctggtgaccttctaagaactgctgttacgggccttacagatgctgagaagcccgtggtcattgagcttgctgttactgcaatggaggaacttataaggatggctcaaactgaagagccattatggttgccaagctcaggctctgagactttatgtgagcaagaatatgctcgtattttccctcgaggccttggacctaagccagctacactcaattctgaagcctcacgagaatctgctgttgtgattatgaatcatatcaatttagttgagattttgatggatgtgaaccaatggactactgtttttgctggtctggtgtcaaaagcaatgactcttgaagtcttatcaactggtgtcgcaggaaatcacaatggagcattgcaagtgatgacagcagaatttcaagttccatctccacttgttccaactcgggagaactatttcttaagatactgtaaacaacatggtgaagggacttgggtagtggttgatgtttccctggacaacttgcgcactgtttcagttccgcgttgcagaagaaggccatctggttgtttaatccaagaaatgccaaatggttactcaagggttatatgggttgaacacgttgaggtggatgaaaatgctgtccatgacatctacaaacctcttgtcaattctgggattgcatttggagcaaaacgctgggtagcaactttagatagacaatgtgaacgccttgcaagtgtgttggcgcttaacatcccaacaggagatgttggaatcattactagtccagctggtcgaaagagtatgctaaaacttgctgagagaatggtgatgagcttttgtgctggagttggtgcatcgacaactcacatatggacaactttgtctggaagtggtgcggatgatgttagagtcatgactaggaagagtatcgatgatccagggagacctcctggtattgtgctgagtgctgcaacatctttttggcttccagtttctcctaagagagtgtttgattttctccgcgatgagaactctagaaatgagtgggatattctttcaaatggtgggattgttcaggaaatggcacacattgcaaatggtcgtgatccaggaaactgtgtttctctactccgtgtcaatactggaacaaactctaaccagagtaacatgctgatactccaagagagcacaactgatgtaacaggatcttacgtcatttacgctccagttgatattgctgcaatgaacgtggtgttaggtgggggtgaccctgactatgttgctctgttgccatctggttttgctattcttccagacggaccgatgaattatcatggtggaggtaattcagaaattgattctcctggtggatcgctactaactgtagcatttcagatattggttgattcagtcccaactgcaaagctttcccttggctctgttgcgactgttaatagtctcatcaaatgcaccgttgaaaagatcaaaggtgctgtaacttccgcaaatgcatga

SEQ ID NO: 13 - DNA, 825 bp, Lycopersicon esculentum
atgaacagtacatctatgtcttcattgggagtgagaaaaggttcatggactgatgaagaagattttcttctaagaaaatgtattgataagtatggtgaaggaaaatggcatcttgttcccataagagctggtctgaatagatgtcggaaaagttgtagattgaggtggctgaattatctaaggccacatatcaagagaggtgactttgaacaagatgaagtggatctcattttgaggcttcataagctcttaggcaacagatggtcacttattgctggtagacttcccggaaggacagctaacgatgtgaaaaactattggaacactaatcttctaaggaagttaaatactactaaaattgttcctcgcgaaaagattaacaataagtgtggagaaattagtactaagattgaaattataaaacctcaacgacgcaagtatttctcaagcacaatgaagaatgttacaaacaataatgtaattttggacgaggaggaacattgcaaggaaataataagtgagaaacaaactccagatgcatcgatggacaacgtagatccatggtggataaatttactggaaaattgcaatgacgatattgaagaagatgaagaggttgtaattaattatgaaaaaacactaacaagtttgttacatgaagaaatatcaccaccattaaatattggtgaaggtaactccatgcaacaaggacaaataagtcatgaaaattggggtgaattttctcttaatttaccacccatgcaacaaggagtacaaaatgatgatttttctgctgaaattgacttatggaatctacttgattaa

SEQ ID NO: 14 - DNA, 825 bp, Artificial Sequence
  SlANT1 with Nicotiana tabacum codon usage
atgaattctacaagtatgtcaagcttaggcgttcgtaagggatcttggacagatgaagaagatttccttctacgaaagtgtattgacaaatatggtgagggaaaatggcatttggttccgattagagctggtttgaatcgatgcaggaaatcctgtagacttaggtggttgaactatcttagacctcacataaagagaggtgatttcgagcaagatgaagtggatctcatactcagactacacaaacttttagggaatcgttggagtcttattgcaggcagattaccaggtagaacagccaatgatgtcaagaactattggaatactaatcttttaaggaagttgaacactacaaagatagtaccaagggagaaaatcaacaacaaatgtggggaaatttctacgaaaattgagattatcaagccccaaagacgtaagtacttttcatccactatgaagaatgtcaccaacaacaatgttatcctcgacgaagaagaacattgcaaagagatcatttctgagaagcagactcctgatgcttcaatggacaacgttgatccttggtggataaatcttctagagaattgcaacgatgatatagaagaggatgaagaagtggtgattaactacgagaaaaccttaactagcctgttgcatgaagaaatctctccaccccttaatattggagaaggaaattcaatgcaacaaggccagatttctcatgagaattggggtgaattttccttgaatctgccacctatgcagcaaggagtacagaatgacgactttagtgcagagattgatctctggaatctgttggactaa

SEQ ID NO: 15 - DNA, 825 bp, Artificial Sequence
  SlANT1 with Arabidopsis thaliana codon usage
atgaattcaacatcaatgtctagtctaggagtaaggaaaggttcatggacagatgaagaggactttcttctccggaaatgcattgataagtatggggaaggaaaatggcatttagtccccattagagctggcttgaatcgttgtaggaaatcgtgtcgactcagatggctaaactatcttagaccgcatatcaagcggggtgatttcgaacaggacgaagtggacttgattttgaggcttcacaagttattgggtaatcgttggtcccttatagctgggagattaccaggtagaacagccaatgatgtgaagaattactggaatacgaacttgctgagaaaactcaacactaccaagatcgttccgagagaaaagatcaacaacaaatgtggcgagattagcacgaagatagagatcataaagcctcaacgtcgaaaatacttctctagcactatgaagaatgtcaccaataacaacgtgatactagatgaagaagaacactgtaaggagattatcagtgagaaacagactcctgatgcatctatggacaatgttgatccttggtggattaaccttctggagaattgcaatgacgatattgaggaggatgaagaggttgtaatcaactatgagaaaacacttacttcactccttcatgaagagatatctccaccacttaacattggagagggtaactccatgcaacaaggacagatctctcatgaaaattggggagaattttcgctgaatttgcctccaatgcaacaaggagttcagaacgacgattttagtgcggaaattgatctctggaacttattggattaa

SEQ ID NO: 16 - DNA, 825 bp, Artificial Sequence
  SlANT1 with Potato Virus X codon usage
atgaatagcactagcatgtcaagcttaggtgtgagaaagggctcatggactgacgaagaggatttcctgttgaggaagtgcatcgacaagtatggagaaggcaaatggcaccttgtaccgattagggcagggcttaacaggtgcaggaaaagctgtaggttgaggtggttgaactatctcagaccccatataaagagaggcgactttgagcaagatgaagtggacctaattcttcgcttacacaaactccttgggaataggtggagtctgatagctggaaggctacctggtagaacagctaacgacgtgaagaactactggaataccaacctattacgcaaactgaacactaccaaaatcgttcccagagagaagatcaacaacaagtgtggcgagataagcacgaagatcgaaatcatcaaaccgcaaagaaggaagtacttcagttcaaccatgaagaatgtcacaaacaacaatgtcatactggatgaagaagagcactgcaaggagattatttccgagaaacagacaccagacgcatccatggacaatgtcgatccatggtggattaacctactcgaaaattgcaacgatgacattgaagaggatgaggaagtagtgatcaactacgagaaaacactgacttctctcttgcatgaggagatcagtccacctttgaacattggagaagggaattctatgcaacaaggacagataagccacgaaaattggggagagttttccctcaatctcccacctatgcaacagggtgttcagaacgatgacttctcagccgaaatcgacttatggaacctactcgactaa

SEQ ID NO: 17 - DNA, 825 bp, Artificial Sequence
  SlANT1 with Homo sapiens codon usage
atgaattctacgtccatgtctagcctcggggttaggaaaggctcatggacagacgaagaggactttctgctgcgcaaatgcatagacaagtatggcgaaggaaagtggcatctggtgcccattagggctggtctgaaccggtgtcgcaagtcctgtaggttgcggtggcttaactacctcagaccccacatcaaacgaggcgatttcgaacaggatgaggtcgacctgattctccgtctgcacaagctgttgggtaacagatggagcctcattgcagggagactccctggaagaactgccaatgacgtcaagaactactggaacaccaaccttcttcgcaagctgaataccactaagatcgttcctcgagagaagatcaacaacaaatgtggagaaatatccaccaaaatcgagatcatcaagccacaacggaggaaatacttctccagcacaatgaagaatgtgaccaacaacaacgtgattttggacgaagaggagcattgcaaagagatcatcagtgagaagcagacacctgatgcctctatggataatgtggacccctggtggataaatctgctggagaattgcaatgatgacattgaagaagatgaggaagtggtcatcaactatgagaaaacactgacttcactgctgcatgaagagattagtccaccgctgaacattggggaggggaatagcatgcagcagggacagatcagtcacgaaaattggggcgaattcagccttaatctcccacccatgcaacagggcgtacagaacgacgacttttcagcggagattgatctgtggaatttgctggattaa

SEQ ID NO: 18 - DNA, 825 bp, Artificial Sequence
  SlANT1 with Oryza sativa codon usage
atgaattcaacgagcatgagctcgttgggtgttcgcaaaggctcttggaccgatgaagaggacttcctcttgcgaaagtgcatcgataagtatggggaaggaaagtggcatcttgtacccatacgtgcgggacttaaccggtgtcgcaagtcgtgcagactcaggtggctcaactatctacggcctcacatcaaacgtggcgatttcgaacaagacgaggttgaccttatcctgagactgcacaaactgctcggcaatcgctggagtctcatagctggtcgattgcctgggaggactgccaatgacgtcaagaattactggaatacaaaccttctgaggaagctgaataccacgaagatagttcctcgggagaagatcaacaacaagtgtggggagatttccacgaaaatcgagatcatcaagccgcaaaggcgcaaatacttctcaagcacaatgaagaacgtcaccaacaacaacgtgattctcgatgaggaggaacactgcaaggagatcatctctgagaaacagactccagatgcctcaatggacaatgtggatccgtggtggattaacctcctggagaactgcaatgatgacattgaagaggacgaagaggtcgtgatcaactacgaaaagaccctcacatctctcctccatgaggaaataagtccaccgctcaatattggcgaaggcaattccatgcagcaaggccagatttcgcatgagaactggggtgagttttccctgaatctaccacccatgcagcaaggagtgcagaatgatgacttttccgcagagattgacttgtggaacttgcttgattaa

SEQ ID NO: 19 - DNA, 825 bp, Artificial Sequence
  SlANT1 with Hordeum vulgare codon usage
atgaatagcacctccatgtcctctctgggcgttcgtaaggggtcatggacagatgaggaggacttcttgctccgcaaatgcatcgacaagtatggcgaaggcaaatggcatcttgtcccgataagggccggactcaaccgctgcagaaagtcttgccgccttaggtggctaaactacctacggccccacattaagcggggtgactttgagcaggatgaggtagacttgatcttgcggctacacaagcttctgggcaataggtggtcactgattgccggtagactccctggtcgcactgcgaatgacgtgaagaactactggaacaccaatctgctccgcaaactcaacaccaccaagatcgtcccacgtgagaagatcaacaacaagtgtggcgagatcagcaccaagatcgagatcatcaagccacaacggaggaagtacttctcctctacgatgaagaatgtgacgaacaacaacgtgattctcgacgaagaggagcactgtaaggagatcatctccgagaaacagactcccgatgcttcgatggacaatgtcgatccgtggtggattaacctcctggagaattgcaacgatgacatagaagaggacgaagaagtcgtgatcaactacgaaaagacgctgacaagcctcttgcacgaggagatatcgccacccctcaacattggagaggggaacagcatgcagcaagggcagatcagtcatgaaaactggggagagttcagcctcaatcttcctccgatgcagcaaggcgttcagaacgatgacttcagtgcagagattgacctgtggaaccttctcgattaa

SEQ ID NO: 20 - DNA, 825 bp, Artificial Sequence
  SlANT1 with Bifidobacterium codon usage
atgaactccacctccatgtcctcgctcggcgttcgcaaaggcagctggaccgatgaggaggacttcctcctgcgcaagtgcatcgacaagtacggagaaggcaaatggcaccttgtccccattcgcgctggtctgaaccgctgtcgcaagagctgccgtttgcggtggctgaactatctgcgtccgcacatcaagcgcggcgacttcgagcaggacgaagtcgacctgattctgcgcctgcataagctgctggggaaccgctggtccctgattgccggccggttgcccggtaggaccgcgaacgacgtgaagaactactggaacaccaacctccttcgcaagctgaataccacgaagatcgtgccgagggagaagatcaacaacaaatgcggggaaatctcgacgaagatcgagatcatcaagccccaacgtcggaagtacttcagcagcaccatgaagaacgtgacgaacaacaacgtgatcctggacgaagaggaacactgcaaggagatcatctcggagaagcagactccggatgcctccatggacaacgtggatccgtggtggatcaatctgctggagaactgcaacgacgacatcgaggaggatgaggaagtcgtgatcaactacgaaaagaccttgacgtccctcctccatgaggagatttcccctccgctgaacatcggcgagggcaactccatgcaacagggccagatctcccacgagaattggggcgaattctcgctgaatctcccgccgatgcagcagggagtccagaacgacgactttagcgccgaaatcgacctctggaaccttctcgattaa

SEQ ID NO: 21 - DNA, 678 bp, Artificial Sequence
  SlLOG1 with Oryza sativa codon usage
atggagaacaaccatcaaacgcagattcagactaccaagacttctcgcttcaagcgcatttgcgtgttctgtgggtcaagtccaggcaagaagccctcctatcagcttgctgccatccagctggggaatcagctggttgaacggaatatcgatctcgtctatggtggaggctctgttggcctaatgggactcgtgagccaatccgtgttcaatggtggtcgacatgtcctcggcgtgataccgaaaaccctgatgcccagagagatcacgggagagtcagtcggagaagtccgggctgtttctggcatgcatcagaggaaagccgagatggcacgtcaagccgatgcgtttatagcgcttcctggcggttacggaaccctcgaagagctactggaggtgattacatgggctcagttgggcatacacgacaaaccagttggcctcttgaacgtggatgggtactacaactcgttgctttcgttcatcgacaaggcagtagacgaggggtttgtgacaccatccgcaagacacatcatcattagtgcgcctacagcccaagaactcatgagcaagcttgaggactatgtcccgaagcacaatggggtagccccgaaactgagctgggagatggaacaacagctcggctacacgactaccaagctcgagattgcgaggtga

SEQ ID NO: 22 - DNA, 1059 bp, Artificial Sequence
  SlOVATE with Oryza sativa codon usage
atgggcaaaagtctcaagctgcgcttttctcgtgtgattgccagcttcaattcgtgcagatctaagaatcccagctcacttccgcaaaatccgaacttctttccccacaagcttacatcgacaaaacacatctctccagactttccgctgattgaccagaaccagaaccagaatcacaggaactacgttcctgagtcgaccatgatcagtgtgggctgttgcagatccgaattcaagtgggagaaagaggagaagtttcacgtggtatcaagctcgttcgtttccgaggaagaggagtgtgaagaagagatcaaccttgctctacgtccaccgctaacaccaccgcgcttctcaaggatagttgtcgagaagaagaagaagaaacagcaacgggtgaagaaaacgaaaaccaaatcccgcatcattcgcatgtccacttcatctgcggatgagtacagtgggatcttgagcggtaccaacacagattgggacaacaatgaggaggaaaccgaaagtctggtgtccagctcaaggagctgttacgacttctcgagtgatgactcgtccacggatttcaatccgcatttggagactatttgcgaaactacgacaatgagaaggcggcataaaaggaatgccaacacgaagcgacgctctatcaaacaaagccgaccttcattctcctcaagcaagggacgcagaagctccgtgtcgacctcctcagactctgagctcccagctaggctcagtgtctttaagaagctcattccttgctctgtggatggaaaggtcaaggagtccttcgcaatcgtcaagaaatcgcaagatccctatgaggacttcaagcggtctatgatggagatgatcctggagaaggaaatgtttgagaagaatgagctcgaacagcttctccagtgcttcctctccctcaacggcaagcattaccatggtgtcatagttgaagcgtttagcgacatatgggaaacgctgttcttggggaataacgatcgggtacgtcgaatgagcattcacgatcctactcccacctattgccggtga

SEQ ID NO: 23 - DNA, 11362 bp, Artificial Sequence
  pNMD674
cttctgtcagcgggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcagatcaggatctcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcgtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgcctgatctggggaaccctgtggttggcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctaagctaggcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagaaaactaaaccatacaccaccaacacaaccaaacccaccacgcccaattgttacacacccgcttgaaaaagaaagtttaacaaatggccaaggtgcgcgaggtttaccaatcttttacagactccaccacaaaaactctcatccaagatgaggcttatagaaacattcgccccatcatggaaaaacacaaactagctaacccttacgctcaaacggttgaagcggctaatgatctagaggggttcggcatagccaccaatccctatagcattgaattgcatacacatgcagccgctaagaccatagagaataaacttctagaggtgcttggttccatcctaccacaagaacctgttacatttatgtttcttaaacccagaaagctaaactacatgagaagaaacccgcggatcaaggacattttccaaaatgttgccattgaaccaagagacgtagccaggtaccccaaggaaacaataattgacaaactcacagagatcacaacggaaacagcatacattagtgacactctgcacttcttggatccgagctacatagtggagacattccaaaactgcccaaaattgcaaacattgtatgcgaccttagttctccccgttgaggcagcctttaaaatggaaagcactcacccgaacatatacagcctcaaatacttcggagatggtttccagtatataccaggcaaccatggtggcggggcataccatcatgaattcgctcatctacaatggctcaaagtgggaaagatcaagtggagggaccccaaggatagctttctcggacatctcaattacacgactgagcaggttgagatgcacacagtgacagtacagttgcaggaatcgttcgcggcaaaccacttgtactgcatcaggagaggagacttgctcacaccggaggtgcgcactttcggccaacctgacaggtacgtgattccaccacagatcttcctcccaaaagttcacaactgcaagaagccgattctcaagaaaactatgatgcagctcttcttgtatgttaggacagtcaaggtcgcaaaaaattgtgacatttttgccaaagtcagacaattaattaaatcatctgacttggacaaatactctgctgtggaactggtttacttagtaagctacatggagttccttgccgatttacaagctaccacctgcttctcagacacactttctggtggcttgctaacaaagacccttgcaccggtgagggcttggatacaagagaaaaagatgcagctgtttggtcttgaggactacgcgaagttagtcaaagcagttgatttccacccggtggatttttctttcaaagtggaaacttgggacttcagattccaccccttgcaagcgtggaaagccttccgaccaagggaagtgtcggatgtagaggaaatggaaagtttgttctcagatggggacctgcttgattgcttcacaagaatgccagcttatgcggtaaacgcagaggaagatttagctgcaatcaggaaaacgcccgagatggatgtcggtcaagaagttaaagagcctgcaggagacagaaatcaatactcaaaccctgcagaaactttcctcaacaagctccacaggaaacacagtagggaggtgaaacaccaggccgcaaagaaagctaaacgcctagctgaaatccaggagtcaatgagagctgaaggtgatgccgaaccaaatgaaataagcgggacgatgggggcaatacccagcaacgccgaacttcctggcacgaatgatgccagacaagaactcacactcccaaccactaaacctgtccctgcaaggtgggaagatgcttcattcacagattctagtgtggaagaggagcaggttaaactccttggaaaagaaaccgttgaaacagcgacgcaacaagtcatcgaaggacttccttggaaacactggattcctcaattaaatgctgttggattcaaggcgctggaaattcagagggataggagtggaacaatgatcatgcccatcacagaaatggtgtccgggctggaaaaagaggacttccctgaaggaactccaaaagagttggcacgagaattgttcgctatgaacagaagccctgccaccatccctttggacctgcttagagccagagactacggcagtgatgtaaagaacaagagaattggtgccatcacaaagacacaggcaacgagttggggcgaatacttgacaggaaagatagaaagcttaactgagaggaaagttgcgacttgtgtcattcatggagctggaggttctggaaaaagtcatgccatccagaaggcattgagagaaattggcaagggctcggacatcactgtagtcctgccgaccaatgaactgcggctagattggagtaagaaagtgcctaacactgagccctatatgttcaagacctctgaaaaggcgttaattgggggaacaggcagcatagtcatctttgacgattactcaaaacttcctcccggttacatagaagccttagtctgtttctactctaaaatcaagctaatcattctaacaggagatagcagacaaagcgtctaccatgaaactgctgaggacgcctccatcaggcatttgggaccagcaacagagtacttctcaaaatactgccgatactatctcaatgccacacaccgcaacaagaaagatcttgcgaacatgcttggtgtctacagtgagagaacgggagtcaccgaaatcagcatgagcgccgagttcttagaaggaatcccaactttggtaccctcggatgagaagagaaagctgtacatgggcaccgggaggaatgacacgttcacatacgctggatgccaggggctaactaagccgaaggtacaaatagtgttggaccacaacacccaagtgtgtagcgcgaatgtgatgtacacggcactttctagagccaccgataggattcacttcgtgaacacaagtgcaaattcctctgccttctgggaaaagttggacagcaccccttacctcaagactttcctatcagtggtgagagaacaagcactcagggagtacgagccggcagaggcagagccaattcaagagcctgagccccagacacacatgtgtgtcgagaatgaggagtccgtgctagaagagtacaaagaggaactcttggaaaagtttgacagagagatccactctgaatcccatggtcattcaaactgtgtccaaactgaagacacaaccattcagttgttttcgcatcaacaagcaaaagatgagactctcctctgggcgactatagatgcgcggctcaagaccagcaatcaagaaacaaacttccgagaattcctgagcaagaaggacattggggacgttctgtttttaaactaccaaaaagctatgggtttacccaaagagcgtattcctttttcccaagaggtctgggaagcttgtgcccacgaagtacaaagcaagtacctcagcaagtcaaagtgcaacttgatcaatgggactgtgagacagagcccagacttcgatgaaaataagattatggtattcctcaagtcgcagtgggtcacaaaggtggaaaaactaggtctacccaagattaagccaggtcaaaccatagcagccttttaccagcagactgtgatgctttttggaactatggctaggtacatgcgatggttcagacaggctttccagccaaaagaagtcttcataaactgtgagacgacgccagatgacatgtctgcatgggccttgaacaactggaatttcagcagacctagcttggctaatgactacacagctttcgaccagtctcaggatggagccatgttgcaatttgaggtgctcaaagccaaacaccactgcataccagaggaaatcattcaggcatacatagatattaagactaatgcacagattttcctaggcacgttatcaattatgcgcctgactggtgaaggtcccacttttgatgcaaacactgagtgcaacatagcttacacccatacaaagtttgacatcccagccggaactgctcaagtttatgcaggagacgactccgcactggactgtgttccagaagtgaagcatagtttccacaggcttgaggacaaattactcctaaagtcaaagcctgtaatcacgcagcaaaagaagggcagttggcctgagttttgtggttggctgatcacaccaaaaggggtgatgaaagacccaattaagctccatgttagcttaaaattggctgaagctaagggtgaactcaagaaatgtcaagattcctatgaaattgatctgagttatgcctatgaccacaaggactctctgcatgacttgttcgatgagaaacagtgtcaggcacacacactcacttgcagaacactaatcaagtcagggagaggcactgtctcactttcccgcctcagaaactttctttaaccgttaagttaccttagagatttgaataagatgtcagcaccagctagtacaacacagcccatagggtcaactacctcaactaccacaaaaactgcaggcgcaactcctgccacagcttcaggcctgttcactatcccggatggggatttctttagtacagcccgtgccatagtagccagcaatgctgtcgcaacaaatgaggacctcagcaagattgaggctatttggaaggacatgaaggtgcccacagacactatggcacaggctgcttgggacttagtcagacactgtgctgatgtaggatcatccgctcaaacagaaatgatagatacaggtccctattccaacggcatcagcagagctagactggcagcagcaattaaagaggtgtgcacacttaggcaattttgcatgaagtatgccccagtggtatggaactggatgttaactaacaacagtccacctgctaactggcaagcacaaggtttcaagcctgagcacaaattcgctgcattcgacttcttcaatggagtcaccaacccagctgccatcatgcccaaagaggggctcatccggccaccgtctgaagctgaaatgaatgctgcccaaactgctgcctttgtgaagattacaaaggccagggcacaatccaacgactttgccagcctagatgcagctgtcactcgaggaaggatcaccggaacgaccacagcagaggcagtcgttactctgcctcctccataacagaaactttctttaaccgttaagttaccttagagatttgaataagatggatattctcatcagtagtttgaaaagtttaggttattctaggacttccaaatctttagattcaggacctttggtagtacatgcagtagccggagccggtaagtccacagccctaaggaagttgatcctcagacacccaacattcaccgtgcatacactcggtgtccctgacaaggtgagtatcagaactagaggcatacagaagccaggacctattcctgagggcaacttcgcaatcctcgatgagtatactttggacaacaccacaaggaactcataccaggcactttttgctgacccttatcaggcaccggagtttagcctagagccccacttctacttggaaacatcatttcgagttccgaggaaagtggcagatttgatagctggctgtggcttcgatttcgagacgaactcaccggaagaagggcacttagagatcactggcatattcaaagggcccctactcggaaaggtgatagccattgatgaggagtctgagacaacactgtccaggcatggtgttgagtttgttaagccctgccaagtgacgggacttgagttcaaagtagtcactattgtgtctgccgcaccaatagaggaaattggccagtccacagctttctacaacgctatcaccaggtcaaagggattgacatatgtccgcgcagggccataggctgaccgctccggtcaattctgaaaaagtgtacatagtattaggtctatcatttgctttagtttcaattacctttctgctttctagaaatagcttaccccacgtcggtgacaacattcacagcttgccacacggaggagcttacagagacggcaccaaagcaatcttgtacaactccccaaatctagggtcacgagtgagtctacacaacggaaagaacgcagcatttgctgccgttttgctactgactttgctgatctatggaagtaaatacatatctcaacgcaatcatacttgtgcttgtggtaacaatcatagcagtcattagcacttccttagtgaggactgaaccttgtgtcatcaagattactggggaatcaatcacagtgttggcttgcaaactagatgcagaaaccataagggccattgccgatctcaagccactctccgttgaacggttaagtttccattgatactcgaaagaggtcagcaccagctagcaacaaacaagaacatgagagacctcgcgatttaaatcgatggtctcagatcggtcgtatcactggaacaacaaccgctgaggctgttgtcactctaccaccaccataactacgtctacataaccgacgcctaccccagtttcatagtattttctggtttgattgtatgaataatataaataaaaaaaaaaaaaaaaaaaaaaaactagtgagct

SEQ ID NO: 24 - DNA, 12380 bp, Artificial Sequence
  pNMD4300
aaactgaaggcgggaaacgacaatctgatctaagctaggcatgcctgcaggtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagaaaactaaaccatacaccaccaacacaaccaaacccaccacgcccaattgttacacacccgcttgaaaaagaaagtttaacaaatggccaaggtgcgcgaggtttaccaatcttttacagactccaccacaaaaactctcatccaagatgaggcttatagaaacattcgccccatcatggaaaaacacaaactagctaacccttacgctcaaacggttgaagcggctaatgatctagaggggttcggcatagccaccaatccctatagcattgaattgcatacacatgcagccgctaagaccatagagaataaacttctagaggtgcttggttccatcctaccacaagaacctgttacatttatgtttcttaaacccagaaagctaaactacatgagaagaaacccgcggatcaaggacattttccaaaatgttgccattgaaccaagagacgtagccaggtaccccaaggaaacaataattgacaaactcacagagatcacaacggaaacagcatacattagtgacactctgcacttcttggatccgagctacatagtggagacattccaaaactgcccaaaattgcaaacattgtatgcgaccttagttctccccgttgaggcagcctttaaaatggaaagcactcacccgaacatatacagcctcaaatacttcggagatggtttccagtatataccaggcaaccatggtggcggggcataccatcatgaattcgctcatctacaatggctcaaagtgggaaagatcaagtggagggaccccaaggatagctttctcggacatctcaattacacgactgagcaggttgagatgcacacagtgacagtacagttgcaggaatcgttcgcggcaaaccacttgtactgcatcaggagaggagacttgctcacaccggaggtgcgcactttcggccaacctgacaggtacgtgattccaccacagatcttcctcccaaaagttcacaactgcaagaagccgattctcaagaaaactatgatgcagctcttcttgtatgttaggacagtcaaggtcgcaaaaaattgtgacatttttgccaaagtcagacaattaattaaatcatctgacttggacaaatactctgctgtggaactggtttacttagtaagctacatggagttccttgccgatttacaagctaccacctgcttctcagacacactttctggtggcttgctaacaaagacccttgcaccggtgagggcttggatacaagagaaaaagatgcagctgtttggtcttgaggactacgcgaagttagtcaaagcagttgatttccacccggtggatttttctttcaaagtggaaacttgggacttcagattccaccccttgcaagcgtggaaagccttccgaccaagggaagtgtcggatgtagaggaaatggaaagtttgttctcagatggggacctgcttgattgcttcacaagaatgccagcttatgcggtaaacgcagaggaagatttagctgcaatcaggaaaacgcccgagatggatgtcggtcaagaagttaaagagcctgcaggagacagaaatcaatactcaaaccctgcagaaactttcctcaacaagctccacaggaaacacagtagggaggtgaaacaccaggccgcaaagaaagctaaacgcctagctgaaatccaggagtcaatgagagctgaaggtgatgccgaaccaaatgaaataagcgggacgatgggggcaatacccagcaacgccgaacttcctggcacgaatgatgccagacaagaactcacactcccaaccactaaacctgtccctgcaaggtgggaagatgcttcattcacagattctagtgtggaagaggagcaggttaaactccttggaaaagaaaccgttgaaacagcgacgcaacaagtcatcgaaggacttccttggaaacactggattcctcaattaaatgctgttggattcaaggcgctggaaattcagagggataggagtggaacaatgatcatgcccatcacagaaatggtgtccgggctggaaaaagaggacttccctgaaggaactccaaaagagttggcacgagaattgttcgctatgaacagaagccctgccaccatccctttggacctgcttagagccagagactacggcagtgatgtaaagaacaagagaattggtgccatcacaaagacacaggcaacgagttggggcgaatacttgacaggaaagatagaaagcttaactgagaggaaagttgcgacttgtgtcattcatggagctggaggttctggaaaaagtcatgccatccagaaggcattgagagaaattggcaagggctcggacatcactgtagtcctgccgaccaatgaactgcggctagattggagtaagaaagtgcctaacactgagccctatatgttcaagacctctgaaaaggcgttaattgggggaacaggcagcatagtcatctttgacgattactcaaaacttcctcccggttacatagaagccttagtctgtttctactctaaaatcaagctaatcattctaacaggagatagcagacaaagcgtctaccatgaaactgctgaggacgcctccatcaggcatttgggaccagcaacagagtacttctcaaaatactgccgatactatctcaatgccacacaccgcaacaagaaagatcttgcgaacatgcttggtgtctacagtgagagaacgggagtcaccgaaatcagcatgagcgccgagttcttagaaggaatcccaactttggtaccctcggatgagaagagaaagctgtacatgggcaccgggaggaatgacacgttcacatacgctggatgccaggggctaactaagccgaaggtacaaatagtgttggaccacaacacccaagtgtgtagcgcgaatgtgatgtacacggcactttctagagccaccgataggattcacttcgtgaacacaagtgcaaattcctctgccttctgggaaaagttggacagcaccccttacctcaagactttcctatcagtggtgagagaacaagcactcagggagtacgagccggcagaggcagagccaattcaagagcctgagccccagacacacatgtgtgtcgagaatgaggagtccgtgctagaagagtacaaagaggaactcttggaaaagtttgacagagagatccactctgaatcccatggtcattcaaactgtgtccaaactgaagacacaaccattcagttgttttcgcatcaacaagcaaaagatgagactctcctctgggcgactatagatgcgcggctcaagaccagcaatcaagaaacaaacttccgagaattcctgagcaagaaggacattggggacgttctgtttttaaactaccaaaaagctatgggtttacccaaagagcgtattcctttttcccaagaggtctgggaagcttgtgcccacgaagtacaaagcaagtacctcagcaagtcaaagtgcaacttgatcaatgggactgtgagacagagcccagacttcgatgaaaataagattatggtattcctcaagtcgcagtgggtcacaaaggtggaaaaactaggtctacccaagattaagccaggtcaaaccatagcagccttttaccagcagactgtgatgctttttggaactatggctaggtacatgcgatggttcagacaggctttccagccaaaagaagtcttcataaactgtgagacgacgccagatgacatgtctgcatgggccttgaacaactggaatttcagcagacctagcttggctaatgactacacagctttcgaccagtctcaggatggagccatgttgcaatttgaggtgctcaaagccaaacaccactgcataccagaggaaatcattcaggcatacatagatattaagactaatgcacagattttcctaggcacgttatcaattatgcgcctgactggtgaaggtcccacttttgatgcaaacactgagtgcaacatagcttacacccatacaaagtttgacatcccagccggaactgctcaagtttatgcaggagacgactccgcactggactgtgttccagaagtgaagcatagtttccacaggcttgaggacaaattactcctaaagtcaaagcctgtaatcacgcagcaaaagaagggcagttggcctgagttttgtggttggctgatcacaccaaaaggggtgatgaaagacccaattaagctccatgttagcttaaaattggctgaagctaagggtgaactcaagaaatgtcaagattcctatgaaattgatctgagttatgcctatgaccacaaggactctctgcatgacttgttcgatgagaaacagtgtcaggcacacacactcacttgcagaacactaatcaagtcagggagaggcactgtctcactttcccgcctcagaaactttctttaaccgttaagttaccttagagatttgaataagatgtcagcaccagctagtacaacacagcccatagggtcaactacctcaactaccacaaaaactgcaggcgcaactcctgccacagcttcaggcctgttcactatcccggatggggatttctttagtacagcccgtgccatagtagccagcaatgctgtcgcaacaaatgaggacctcagcaagattgaggctatttggaaggacatgaaggtgcccacagacactatggcacaggctgcttgggacttagtcagacactgtgctgatgtaggatcatccgctcaaacagaaatgatagatacaggtccctattccaacggcatcagcagagctagactggcagcagcaattaaagaggtgtgcacacttaggcaattttgcatgaagtatgccccagtggtatggaactggatgttaactaacaacagtccacctgctaactggcaagcacaaggtttcaagcctgagcacaaattcgctgcattcgacttcttcaatggagtcaccaacccagctgccatcatgcccaaagaggggctcatccggccaccgtctgaagctgaaatgaatgctgcccaaactgctgcctttgtgaagattacaaaggccagggcacaatccaacgactttgccagcctagatgcagctgtcactcgaggaaggatcaccggaacgaccacagcagaggcagtcgttactctgcctcctccataacagaaactttctttaaccgttaagttaccttagagatttgaataagatggatattctcatcagtagtttgaaaagtttaggttattctaggacttccaaatctttagattcaggacctttggtagtacatgcagtagccggagccggtaagtccacagccctaaggaagttgatcctcagacacccaacattcaccgtgcatacactcggtgtccctgacaaggtgagtatcagaactagaggcatacagaagccaggacctattcctgagggcaacttcgcaatcctcgatgagtatactttggacaacaccacaaggaactcataccaggcactttttgctgacccttatcaggcaccggagtttagcctagagccccacttctacttggaaacatcatttcgagttccgaggaaagtggcagatttgatagctggctgtggcttcgatttcgagacgaactcaccggaagaagggcacttagagatcactggcatattcaaagggcccctactcggaaaggtgatagccattgatgaggagtctgagacaacactgtccaggcatggtgttgagtttgttaagccctgccaagtgacgggacttgagttcaaagtagtcactattgtgtctgccgcaccaatagaggaaattggccagtccacagctttctacaacgctatcaccaggtcaaagggattgacatatgtccgcgcagggccataggctgaccgctccggtcaattctgaaaaagtgtacatagtattaggtctatcatttgctttagtttcaattacctttctgctttctagaaatagcttaccccacgtcggtgacaacattcacagcttgccacacggaggagcttacagagacggcaccaaagcaatcttgtacaactccccaaatctagggtcacgagtgagtctacacaacggaaagaacgcagcatttgctgccgttttgctactgactttgctgatctatggaagtaaatacatatctcaacgcaatcatacttgtgcttgtggtaacaatcatagcagtcattagcacttccttagtgaggactgaaccttgtgtcatcaagattactggggaatcaatcacagtgttggcttgcaaactagatgcagaaaccataagggccattgccgatctcaagccactctccgttgaacggttaagtttccattgatactcgaaagaggtcagcaccagctagcaacaaacaagaacatgagagacctcgcgatttaaatcgatggtctcagatcggtcgtatcactggaacaacaaccgctgaggctgttgtcactctaccaccaccataactacgtctacataaccgacgcctaccccagtttcatagtattttctggtttgattgtatgaataatataaataaaaaaaaaaaaaaaaaaaaaaaactagtgagctcttctgtcagcgggcccactgcatccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcagatcaggatctcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcctgtcgatcagatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagtgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcggatccgtcaagccctcaactgataaaaacagcaagaggtgccggttatttctttgacgcggacgtgcaggtttcgcacggggggacgatggcagcctaagatcgacaggctggccaattcgtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgcctgatctggggaaccctgtggttggcacatacaaatggacgaacggataaaccttttcacgcccttttaaatatccgattattctaataaacgctcttttctcttaggtttacccgccaatatatcctgtcaaacactgatagttt

SEQ ID NO: 25 - DNA, 33 bp, Artificial Sequence
  oligonucleotide
tttgaagacatctcaacgcaatcatacttgtgc

SEQ ID NO: 26 - DNA, 38 bp, Artificial Sequence
  oligonucleotide
tttgaagacttctcggttatgtagacgtagttatggtg

SEQ ID NO: 27 - DNA, 11 bp, Artificial Sequence
  BsaI cleavage site
  misc_feature (7)..(11): n is a, c, g, or t
ggtctcnnnnn

SEQ ID NO: 28 - DNA, 13 bp, Artificial Sequence
  donor/acceptor splicing site
aggtraggcaggt

SEQ ID NO: 29 - DNA, 681 bp, Potato virus X
atggatattctcatcagtagtttgaaaagtttaggttattctaggacttccaaatctttagattcaggacctttggtagtacatgcagtagccggagccggtaagtccacagccctaaggaagttgatcctcagacacccaacattcaccgtgcatacactcggtgtccctgacaaggtgagtatcagaactagaggcatacagaagccaggacctattcctgagggcaacttcgcaatcctcgatgagtatactttggacaacaccacaaggaactcataccaggcactttttgctgacccttatcaggcaccggagtttagcctagagccccacttctacttggaaacatcatttcgagttccgaggaaagtggcagatttgatagctggctgtggcttcgatttcgagacgaactcaccggaagaagggcacttagagatcactggcatattcaaagggcccctactcggaaaggtgatagccattgatgaggagtctgagacaacactgtccaggcatggtgttgagtttgttaagccctgccaagtgacgggacttgagttcaaagtagtcactattgtgtctgccgcaccaatagaggaaattggccagtccacagctttctacaacgctatcaccaggtcaaagggattgacatatgtccgcgcagggccatag

SEQ ID NO: 30 - protein, 226 aa, Potato virus X
MDILISSLKSLGYSRTSKSLDSGPLVVHAVAGAGKSTALRKLILRHPTFTVHTLGVPDKVSIRTRGIQKPGPIPEGNFAILDEYTLDNTTRNSYQALFADPYQAPEFSLEPHFYLETSFRVPRKVADLIAGCGFDFETNSPEEGHLEITGIFKGPLLGKVIAIDEESETTLSRHGVEFVKPCQVTGLEFKVVTIVSAAPIEEIGQSTAFYNAITRSKGLTYVRAGP

SEQ ID NO: 31 - DNA, 345 bp, Potato virus X
atgtccgcgcagggccataggctgaccgctccggtcaattctgaaaaagtgtacatagtattaggtctatcatttgctttagtttcaattacctttctgctttctagaaatagcttaccccacgtcggtgacaacattcacagcttgccacacggaggagcttacagagacggcaccaaagcaatcttgtacaactccccaaatctagggtcacgagtgagtctacacaacggaaagaacgcagcatttgctgccgttttgctactgactttgctgatctatggaagtaaatacatatctcaacgcaatcatacttgtgcttgtggtaacaatcatagcagtcat

SEQ ID NO: 32 - protein, 115 aa, Potato virus X
MSAQGHRLTAPVNSEKVYIVLGLSFALVSITFLLSRNSLPHVGDNIHSLPHGGAYRDGTKAILYNSPNLGSRVSLHNGKNAAFAAVLLLTLLIYGSKYISQRNHTCACGNNHSSH

SEQ ID NO: 33 - DNA, 210 bp, Potato virus X
atggaagtaaatacatatctcaacgcaatcatacttgtgcttgtggtaacaatcatagcagtcattagcacttccttagtgaggactgaaccttgtgtcatcaagattactggggaatcaatcacagtgttggcttgcaaactagatgcagaaaccataagggccattgccgatctcaagccactctccgttgaacggttaagtttccat

SEQ ID NO: 34 - protein, 70 aa, Potato virus X
MEVNTYLNAIILVLVVTIIAVISTSLVRTEPCVIKITGESITVLACKLDAETIRAIADLKPLSVERLSFH

SEQ ID NO: 35 - DNA, 714 bp, Potato virus X
atgtcagcaccagctagcacaacacagcccatagggtcaactacctcaactaccacaaaaactgcaggcgcaactcctgccacagcttcaggcctgttcactatcccggatggggatttctttagtacagcccgtgccatagtagccagcaatgctgtcgcaacaaatgaggacctcagcaagattgaggctatttggaaggacatgaaggtgcccacagacactatggcacaggctgcttgggacttagtcagacactgtgctgatgtaggatcatccgctcaaacagaaatgatagatacaggtccctattccaacggcatcagcagagctagactggcagcagcaattaaagaggtgtgcacacttaggcaattttgcatgaagtatgccccagtggtatggaactggatgttaactaacaacagtccacctgctaactggcaagcacaaggtttcaagcctgagcacaaattcgctgcattcgacttcttcaatggagtcaccaacccagctgccatcatgcccaaagaggggctcatccggccaccgtctgaagctgaaatgaatgctgcccaaactgctgcctttgtgaagattacaaaggccagggcacaatccaacgactttgccagcctagatgcagctgtcactcgaggtcgtatcactggaacaacaaccgctgaggctgttgtcactctaccaccaccataa

SEQ ID NO: 36 - protein, 237 aa, Potato virus X
MSAPASTTQPIGSTTSTTTKTAGATPATASGLFTIPDGDFFSTARAIVASNAVATNEDLSKIEAIWKDMKVPTDTMAQAAWDLVRHCADVGSSAQTEMIDTGPYSNGISRARLAAAIKEVCTLRQFCMKYAPVVWNWMLTNNSPPANWQAQGFKPEHKFAAFDFFNGVTNPAAIMPKEGLIRPPSEAEMNAAQTAAFVKITKARAQSNDFASLDAAVTRGRITGTTTAEAVVTLPPP

SEQ ID NO: 37 - DNA, 4371 bp, Potato virus X
atggccaaggtgcgcgaggtttaccaatcttttacagactccaccacaaaaactctcatccaagatgaggcttatagaaacattcgccccatcatggaaaaacacaaactagctaacccttacgctcaaacggttgaagcggctaatgatctagaggggttcggcatagccaccaatccctatagcattgaattgcatacacatgcagccgctaagaccatagagaataaacttctagaggtgcttggttccatcctaccacaagaacctgttacatttatgtttcttaaacccagaaagctaaactacatgagaagaaacccgcggatcaaggacattttccaaaatgttgccattgaaccaagagacgtagccaggtaccccaaggaaacaataattgacaaactcacagagatcacaacggaaacagcatacattagtgacactctgcacttcttggatccgagctacatagtggagacattccaaaactgcccaaaattgcaaacattgtatgcgaccttagttctccccgttgaggcagcctttaaaatggaaagcactcacccgaacatatacagcctcaaatacttcggagatggtttccagtatataccaggcaaccatggtggcggggcataccatcatgaattcgctcatctacaatggctcaaagtgggaaagatcaagtggagggaccccaaggatagctttctcggacatctcaattacacgactgagcaggttgagatgcacacagtgacagtacagttgcaggaatcgttcgcggcaaaccacttgtactgcatcaggagaggagacttgctcacaccggaggtgcgcactttcggccaacctgacaggtacgtgattccaccacagatcttcctcccaaaagttcacaactgcaagaagccgattctcaagaaaactatgatgcagctcttcttgtatgttaggacagtcaaggtcgcaaaaaattgtgacatttttgccaaagtcagacaattaattaaatcatctgacttggacaaatactctgctgtggaactggtttacttagtaagctacatggagttccttgccgatttacaagctaccacctgcttctcagacacactttctggtggcttgctaacaaagacccttgcaccggtgagggcttggatacaagagaaaaagatgcagctgtttggtcttgaggactacgcgaagttagtcaaagcagttgatttccacccggtggatttttctttcaaagtggaaacttgggacttcagattccaccccttgcaagcgtggaaagccttccgaccaagggaagtgtcggatgtagaggaaatggaaagtttgttctcagatggggacctgcttgattgcttcacaagaatgccagcttatgcggtaaacgcagaggaagatttagctgcaatcaggaaaacgcccgagatggatgtcggtcaagaagttaaagagcctgcaggagacagaaatcaatactcaaaccctgcagaaactttcctcaacaagctccacaggaaacacagtagggaggtgaaacaccaggccgcaaagaaagctaaacgcctagctgaaatccaggagtcaatgagagctgaaggtgatgccgaaccaaatgaaataagcgggacgatgggggcaatacccagcaacgccgaacttcctggcacgaatgatgccagacaagaactcacactcccaaccactaaacctgtccctgcaaggtgggaagatgcttcattcacagattctagtgtggaagaggagcaggttaaactccttggaaaagaaaccgttgaaacagcgacgcaacaagtcatcgaaggacttccttggaaacactggattcctcaattaaatgctgttggattcaaggcgctggaaattcagagggataggagtggaacaatgatcatgcccatcacagaaatggtgtccgggctggaaaaagaggacttccctgaaggaactccaaaagagttggcacgagaattgttcgctatgaacagaagccctgccaccatccctttggacctgcttagagccagagactacggcagtgatgtaaagaacaagagaattggtgccatcacaaagacacaggcaacgagttggggcgaatacttgacaggaaagatagaaagcttaactgagaggaaagttgcgacttgtgtcattcatggagctggaggttctggaaaaagtcatgccatccagaaggcattgagagaaattggcaagggctcggacatcactgtagtcctgccgaccaatgaactgcggctagattggagtaagaaagtgcctaacactgagccctatatgttcaagacctctgaaaaggcgttaattgggggaacaggcagcatagtcatctttgacgattactcaaaacttcctcccggttacatagaagccttagtctgtttctactctaaaatcaagctaatcattctaacaggagatagcagacaaagcgtctaccatgaaactgctgaggacgcctccatcaggcatttgggaccagcaacagagtacttctcaaaatactgccgatactatctcaatgccacacaccgcaacaagaaagatcttgcgaacatgcttggtgtctacagtgagagaacgggagtcaccgaaatcagcatgagcgccgagttcttagaaggaatcccaactttggtaccctcggatgagaagagaaagctgtacatgggcaccgggaggaatgacacgttcacatacgctggatgccaggggctaactaagccgaaggtacaaatagtgttggaccacaacacccaagtgtgtagcgcgaatgtgatgtacacggcactttctagagccaccgataggattcacttcgtgaacacaagtgcaaattcctctgccttctgggaaaagttggacagcaccccttacctcaagactttcctatcagtggtgagagaacaagcactcagggagtacgagccggcagaggcagagccaattcaagagcctgagccccagacacacatgtgtgtcgagaatgaggagtccgtgctagaagagtacaaagaggaactcttggaaaagtttgacagagagatccactctgaatcccatggtcattcaaactgtgtccaaactgaagacacaaccattcagttgttttcgcatcaacaagcaaaagatgagactctcctctgggcgactatagatgcgcggctcaagaccagcaatcaagaaacaaacttccgagaattcctgagcaagaaggacattggggacgttctgtttttaaactaccaaaaagctatgggtttacccaaagagcgtattcctttttcccaagaggtctgggaagcttgtgcccacgaagtacaaagcaagtacctcagcaagtcaaagtgcaacttgatcaatgggactgtgagacagagcccagacttcgatgaaaataagattatggtattcctcaagtcgcagtgggtcacaaaggtggaaaaactaggtctacccaagattaagccaggtcaaaccatagcagccttttaccagcagactgtgatgctttttggaactatggctaggtacatgcgatggttcagacaggctttccagccaaaagaagtcttcataaactgtgagacgacgccagatgacatgtctgcatgggccttgaacaactggaatttcagcagacctagcttggctaatgactacacagctttcgaccagtctcaggatggagccatgttgcaatttgaggtgctcaaagccaaacaccactgcataccagaggaaatcattcaggcatacatagatattaagactaatgcacagattttcctaggcacgttatcaattatgcgcctgactggtgaaggtcccacttttgatgcaaacactgagtgcaacatagcttacacccatacaaagtttgacatcccagccggaactgctcaagtttatgcaggagacgactccgcactggactgtgttccagaagtgaagcatagtttccacaggcttgaggacaaattactcctaaagtcaaagcctgtaatcacgcagcaaaagaagggcagttggcctgagttttgtggttggctgatcacaccaaaaggggtgatgaaagacccaattaagctccatgttagcttaaaattggctgaagctaagggtgaactcaagaaatgtcaagattcctatgaaattgatctgagttatgcctatgaccacaaggactctctgcatgacttgttcgatgagaaacagtgtcaggcacacacactcacttgcagaacactaatcaagtcagggagaggcactgtctcactttcccgcctcagaaactttctttaa

SEQ ID NO: 38 - DNA, 720 bp, Artificial Sequence
  coding sequence of sGFP with Nicotiana tabacum codon usage
atggtctcaaaaggagaagagttgtttacaggtgttgttcccattctagtggagttagatggcgatgtgaatggacataagttttccgttagtggtgaaggcgaaggagatgcaacatatgggaaattgacactcaagtttatctgtactacagggaaattaccagttccatggcctacattggtcactaccttttcttatggtgtgcaatgctttagcagatatccagatcacatgaagcaacatgacttctttaagtctgctatgcctgaaggctatgttcaggagagaaccattttcttcaaggatgatggtaactataaaacgagagctgaggtaaagtttgaaggagacactcttgttaatcgaatagaactgaaaggaattgacttcaaggaagatggcaatatacttggtcacaaacttgagtacaactacaatagtcacaatgtgtacattatggcggacaaacagaagaatgggatcaaagtcaacttcaagataaggcacaatatcgaagatggatctgtgcaacttgcagaccattaccaacagaacactccgattggagatggacctgtactattgccagataaccattatctctctactcaatcagccttgtccaaagaccctaatgagaaacgtgatcatatggtactgttagagtttgttaccgcagctggtattactcatggtatggatgaactttacaagtaa